(19) United States
(12) Patent Application Publication
(10) Pub. No.: US 2025/0354188 A1
(43) Pub. Date: Nov. 20, 2025

(54) METHOD FOR SYNTHESIZING CELOSIANIN II, METHOD FOR SYNTHESIZING BETAXANTHIN, AMYLOID-ß POLYMERIZATION INHIBITOR OR THERAPEUTIC OR PROPHYLACTIC AGENT FOR ALZHEIMER'S DISEASE, AMYLOID PEPTIDE AGGREGATION INHIBITOR, AND HIV-1 PROTEASE ACTIVITY INHIBITOR

(71) Applicant: Masashi MORI, Ishikawa (JP)

(72) Inventors: Masashi MORI, Ishikawa (JP); Tomohiro IMAMURA, Ishikawa (JP); Izumi TAKIZAKI, Ishikawa (JP); Akiko MIZUNO, Ishikawa (JP); Yasuki HIGASHIMURA, Ishikawa (JP); Hironori KOGA, Ishikawa (JP); Hiroharu MIZUKOSHI, Ishikawa (JP)

(73) Assignee: Masashi MORI, Ishikawa (JP)

(21) Appl. No.: 19/266,206

(22) Filed: Jul. 11, 2025

Related U.S. Application Data
(62) Division of application No. 17/785,922, filed on Jun. 16, 2022.

(30) Foreign Application Priority Data
Dec. 19, 2019 (JP) 2019-229647
Jul. 11, 2020 (JP) 2020-119563

(51) Int. Cl. C12P 19/60; C12N 9/02; C12N 9/10; C12N 15/82

(52) U.S. Cl. CPC C12P 19/60; C12N 9/0069; C12N 9/0071; C12N 9/1029; C12N 9/1051; C12N 15/825

(57) ABSTRACT

The present invention provides a method of synthesizing celosianin II, a method of synthesizing a betaxanthin, an amyloid-β polymerization inhibitor or a therapeutic or preventive agent for Alzheimer's, an amyloid peptide aggregation inhibitor, and an HIV-1 protease activity inhibitor. A gene having a celosianin II synthesis ability has been isolated from quinoa, and a method of synthesizing celosianin II of the present invention has been constructed. Besides, it has been recognized that celosianin II or the like serves as an active ingredient of each of an amyloid-β polymerization inhibitor or a therapeutic or preventive agent for Alzheimer's, an amyloid peptide aggregation inhibitor, and an HIV-1 protease activity inhibitor.

### TECHNICAL FIELD

The present application is divisional of U.S. patent application Ser. No. 17/785,922, filed Jun. 16, 2022, which is a National Stage Application of PCT/JP2020/047581, filed Dec. 19, 2020, which claims priority to Japanese Patent Application No. 2020-119563 and priority of Japanese Patent Application No. 2019-229647, which are incorporated herein by reference.

### REFERENCE TO AN ELECTRONIC SEQUENCE LISTING

The contents of the electronic sequence listing (revised_3190_256_01--sequencelisting.xml; Size: 122,397 bytes; and Date of Creation: Aug. 14, 2025) is herein incorporated by reference in its entirety.

The present invention relates to a method of synthesizing celosianin II, a method of synthesizing a betaxanthin, an amyloid-β polymerization inhibitor or a therapeutic or preventive agent for Alzheimer's, an amyloid peptide aggregation inhibitor, and an HIV-1 protease activity inhibitor.

### BACKGROUND ART

### Betalains

Betalains, which are a group of plant pigments, are produced in plants of the order Caryophyllales and some fungi. The betalain pigments are broadly classified into two groups, i.e., betacyanins, which range from red to violet in color, and betaxanthins, which range from yellow to orange in color. In plants, the betalain pigments are known to be involved in resistance to environmental stresses (Non Patent Literatures 1 and 2). The betalain pigments are used for food additives because of their vivid colors.

In addition, the betalain pigments have high antioxidant activity, and hence are expected to be utilized as pharmaceuticals and supplements. Previous studies have reported that the betalain pigments have an anti-inflammatory action, an anti-cancer action, and physiological actions, such as suppression of oxidation of LDL cholesterol and inhibition of HIV-1 protease activity (Non Patent Literatures 3 to 9 and FIG. 1).

The betalain pigments, though having various physiological actions, are difficult to produce because the production of the betalain pigments is mainly performed through extraction from betalain pigment-producing plants. In recent years, it has been reported that betalains have been successfully produced by introducing betalain biosynthesis genes into non-betalain-producing plants (Non Patent Literature 2). The inventors of the present invention have also succeeded in synthesizing amaranthin, which is a betalain pigment (Non Patent Literature 4). However, of the betalain pigments, of which many kinds exist, betalain pigments that can be artificially synthesized are limited, and physiological activities of few individual betalain pigments have been revealed.

### Quinoa

Quinoa (Chenopodium quinoa) is a plant belonging to the genus Chenopodium of the subfamily Chenopodioideae of the family Amaranthaceae, and has rich nutrients and excellent environmental stress tolerance. It is known that three kinds of betalains (betanin, amaranthin, and celosianin II) are synthesized and accumulated in hypocotyls of quinoa (FIG. 1 and FIG. 2). The inventors of the present invention have succeeded in synthesizing betanin and amaranthin in tobacco BYII cells (Non Patent Literature 4).

With regard to celosianin II, no synthesis gene has been identified, and an artificial production system in a non-betalain-producing plant has not been constructed. Further, characteristics and physiological activity of celosianin II have not been reported and are unknown (FIG. 1).

In Patent Literature 1, there is a disclosure of a “method of producing a betacyanin, including a step (conversion step) of converting a raw material into a betacyanin in an aqueous medium in the presence of a microorganism having enzyme activity of hydroxylating the 3-position of the phenol ring of tyrosine, DOPA 4,5-dioxygenase activity, L-DOPA oxidase activity, and enzyme activity of glycosylating a phenolic hydroxy group, or a treated product thereof.”

However, a celosianin II synthetase gene and a gene required for betaxanthin synthesis of the present invention are not disclosed or suggested therein.

### CITATION LIST

### Patent Literature

[PTL 1] JP 2016-182044 A

### Non Patent Literature

[NPL 1] Jain, G., Schwinn, K. E., and Gould, K. S. (2015) Betalain induction byl-DOPA application confers photoprotection to saline-exposed leaves of Disphymaaustrale. New Phytol. 207, 1075-1083.
[NPL 2] Polturak, G., Grossman, N., Vela-Corcia, D., Dong, Y., Nudel, A., Pliner, M., Levy, M., Rogachev, I. and Aharoni, A. (2017) Engineered gray mold resistance, antioxidant capacity, and pigmentation in betalain-producing crops and ornamentals. Proc. Natl. Acad. Sci. U. S. A. 114, 9062-9067.
[NPL 3] Kapadia, G. J., Tokuda, H., Konoshima, T. and Nishino, H. (1996) Chemoprevention of lung and skin cancer by Beta vulgaris (beet) root extract. Cancer Lett. 100, 211-214.
[NPL 4] Imamura, T. et al. (2019) Isolation of amaranthin synthetase from Chenopodium quinoa and construction of an amaranthin production system using suspension-cultured tobacco BY-2 cells. Plant Biotechnol J 17, 969-981.
[NPL 5] Lechner, J. F., Wang, L. S., Rocha, C. M., Larue, B., Henry, C., McIntyre, C. M., Riedl, K. M., Schwartz, S. J. and Stoner, G. D. (2010) Drinking water with red beetroot food color antagonizes esophageal carcinogenesis in N-nitrosomethylbenzylamine-treated rats. J. Med. Food 13, 733-739.

[NPL 6] Martinez, R. M., Longhi-Balbinot, D. T., Zarpelon, A. C., Staurengo-Ferrari, L., Baracat, M. M., Georgetti, S. R., Sassonia, R. C., Verri, W .A., Jr. and Casagrande, R. (2015) Anti-inflammatory activity of betalain-rich dye of Beta vulgaris: effect on edema, leukocyte recruitment, superoxide anion and cytokine production. Arch. Pharm. Res. 38, 494-504.

[NPL 7] Rodriguez, E. B., Vidallon, M. L., Mendoza, D. J. and Reyes, C. T. (2016) Health-promoting bioactivities of betalains from red dragon fruit (Hylocereus polyrhizus (Weber) Britton and Rose) peels as affected by carbohydrate encapsulation. J. Sci. Food Agric. 96, 4679-4689.

[NPL 8] Tesoriere, L., Butera, D., D'Arpa, D., DiGaudio, F., Allegra, M., Gentile, C., and Livrea, M. A. (2003) Increased resistance to oxidation of betalain-enriched human low density lipoproteins. Free Radic. Res. 37, 689-696.

[NPL 9] Tesoriere, L., Allegra, M., Butera, D. and Livrea, M. A. (2004) Absorption, excretion, and distribution of dietary antioxidant betalains in LDLs: potential health effects of betalains in humans. Am. J. Clin. Nutr. 80, 941-945.

### SUMMARY OF INVENTION

### Technical Problem

An object of the present invention is to provide a method of synthesizing celosianin II, a method of synthesizing a betaxanthin, an amyloid-β polymerization inhibitor or a therapeutic or preventive agent for Alzheimer's, an amyloid peptide aggregation inhibitor, and an HIV-1 protease activity inhibitor.

### Solution to Problem

In order to achieve the above-mentioned object, the inventors of the present invention have isolated a gene having a celosianin II synthesis ability from quinoa, and have constructed a method of synthesizing celosianin II of the present invention.

Further, in order to achieve the above-mentioned object, the inventors of the present invention have isolated a gene required for betaxanthin synthesis from quinoa, and have constructed a method of synthesizing a betaxanthin of the present invention.

Besides, the inventors have recognized that celosianin II or the like serves as an active ingredient of each of an amyloid-β polymerization inhibitor or a therapeutic or preventive agent for Alzheimer's, an amyloid peptide aggregation inhibitor, and an HIV-1 protease activity inhibitor.

That is, the present invention is as described below.

1. A method of synthesizing celosianin II, including:
culturing a host that has introduced therein the following gene encoding a celosianin II synthetase, a gene encoding an enzyme having activity of hydroxylating a 3-position of a phenol ring of tyrosine, a gene encoding an enzyme having L-DOPA oxidase activity and/or a gene encoding an enzyme having DOPA 4,5-dioxygenase activity, a gene encoding an enzyme having activity of glycosylating a phenolic hydroxy group, and a gene encoding an enzyme having activity of adding glucuronic acid, and that has an ability to produce tyrosine or 3-hydroxy-L-tyrosine (L-DOPA), and extracting celosianin II from the host after the culturing; or
culturing a host that has introduced therein the following gene encoding a celosianin II synthetase, and that has enzyme activity of having activity of hydroxylating a 3-position of a phenol ring of tyrosine, L-DOPA oxidase activity and/or DOPA 4,5-dioxygenase activity, enzyme activity of having activity of glycosylating a phenolic hydroxy group, enzyme activity of having activity of adding glucuronic acid, and an ability to produce tyrosine or 3-hydroxy-L-tyrosine, and extracting celosianin II from the host after the culturing,
wherein the gene encoding a celosianin II synthetase is any one or more selected from the following:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
   (5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

2. A method of synthesizing celosianin II, including culturing a host that has introduced therein the following gene encoding a celosianin II synthetase and that has an ability to produce amaranthin, and extracting celosianin II from the host after the culturing,
wherein the gene encoding a celosianin II synthetase is any one or more selected from the following:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

3. A method of synthesizing celosianin II, including:
culturing a host that has introduced therein the following gene encoding a celosianin II synthetase and a gene encoding an enzyme having activity of adding glucuronic acid, and that has an ability to produce betanin, and extracting celosianin II from the host after the culturing; or
culturing a host that has introduced therein the following gene encoding a celosianin II synthetase, and that has enzyme activity of having activity of adding glucuronic acid and an ability to produce betanin, and extracting celosianin II from the host after the culturing,
wherein the gene encoding a celosianin II synthetase is any one or more selected from the following:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

4. A method of synthesizing celosianin II, including:
culturing a host that has introduced therein the following gene encoding a celosianin II synthetase, a gene encoding an enzyme having activity of glycosylating a phenolic hydroxy group, and a gene encoding an enzyme having activity of adding glucuronic acid, and that has an ability to produce betanidin, and extracting celosianin II from the host after the culturing; or
culturing a host that has introduced therein the following gene encoding a celosianin II synthetase, and that has enzyme activity of having activity of glycosylating a phenolic hydroxy group, enzyme activity of having activity of adding glucuronic acid, and an ability to produce betanidin, and extracting celosianin II from the host after the culturing,
wherein the gene encoding a celosianin II synthetase is any one or more selected from the following:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

5. The method of synthesizing celosianin II according to any one of the above-mentioned items 1 to 4, wherein the gene encoding a celosianin II synthetase is any one or more selected from genes formed of DNAs formed of the base sequences set forth in SEQ ID NOS: 56, 58, 60, 62, 64, 66, 68, 1, 3, 5, 7, 9, 11, 13, and 15.

6. A celosianin II synthesis composition, including a gene shown in any one of the following items or a vector carrying the gene:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15; and
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15.

7. A celosianin II synthesis composition, including a peptide represented by any one of the following amino acid sequences:
(1) an amino acid sequence set forth in SEQ ID NO: 57, 59, 61, 63, 65, 67, 69, 2, 4, 6, 8, 10, 12, 14, or 16;
(2) an amino acid sequence that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that forms a polypeptide having a substantially equivalent ability to synthesize celosianin II to that of the amino acid sequence set forth in SEQ ID NO: 57, 59, 61, 63, 65, 67, 69, 2, 4, 6, 8, 10, 12, 14, or 16; and
(3) an amino acid sequence that has 90% or more homology to the amino acid sequence set forth in SEQ ID NO: 57, 59, 61, 63, 65, 67, 69, 2, 4, 6, 8, 10, 12, 14, or 16, and that forms a polypeptide having a substantially equivalent ability to synthesize celosianin II to that of the amino acid sequence set forth in SEQ ID NO: 57, 59, 61, 63, 65, 67, 69, 2, 4, 6, 8, 10, 12, 14, or 16.

8. A celosianin II-producing host having introduced therein the synthesis composition of the above-mentioned item 6 or 7.

9. A method of synthesizing celosianin II, including the following (1) or (2):
(1) a step of bringing amaranthin into contact with the celosianin II synthesis composition of the above-mentioned item 7; or
(2) (a) a step of bringing betanin into contact with an enzyme having activity of adding glucuronic acid, and
   (b) a step of bringing amaranthin obtained in the step (a) into contact with the celosianin II synthesis composition of the above-mentioned item 7.

10. A celosianin II-producing host,
the host having introduced therein the following gene encoding a celosianin II synthetase, a gene encoding an enzyme having activity of hydroxylating a 3-position of a phenol ring of tyrosine, a gene encoding an enzyme having L-DOPA oxidase activity and/or a gene encoding an enzyme having DOPA 4,5-dioxygenase activity, a gene encoding an enzyme having activity of glycosylating a phenolic hydroxy group, and a gene encoding an enzyme having activity of adding glucuronic acid, and having an ability to produce tyrosine or L-DOPA, or
the host having introduced therein the following gene encoding a celosianin II synthetase, and having enzyme activity of having activity of hydroxylating a 3-position of a phenol ring of tyrosine, L-DOPA oxidase activity and/or DOPA 4,5-dioxygenase activity, enzyme activity of having activity of glycosylating a phenolic hydroxy group, enzyme activity of having activity of adding glucuronic acid, and an ability to produce tyrosine or 3-hydroxy-L-tyrosine,
wherein the gene encoding a celosianin II synthetase is any one or more selected from the following:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, or 12;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

11. A method of synthesizing a betaxanthin, including:
culturing, with addition of an amine, a host that has introduced therein a gene encoding an enzyme having enzyme activity of hydroxylating a 3-position of a phenol ring of tyrosine but being free of L-DOPA oxidase activity, and a gene encoding an enzyme having DOPA 4,5-dioxygenase activity, and that has an ability to produce tyrosine or 3-hydroxy-L-tyrosine, and extracting a betaxanthin from the host after the culturing; or
culturing, with addition of an amine, a host that has introduced therein a gene encoding an enzyme having enzyme activity of hydroxylating a 3-position of a phenol ring of tyrosine but being free of L-DOPA oxidase activity, and that has enzyme activity of having DOPA 4,5-dioxygenase activity, and an ability to produce tyrosine or 3-hydroxy-L-tyrosine, and extracting a betaxanthin from the host after the culturing; or
culturing a host that has introduced therein a gene encoding an enzyme having enzyme activity of hydroxylating a 3-position of a phenol ring of tyrosine but being free of L-DOPA oxidase activity, a gene encoding an enzyme having DOPA 4,5-dioxygenase activity, and an amine synthesis gene, and that has an ability to produce tyrosine or 3-hydroxy-L-tyrosine, and extracting a betaxanthin from the host after the culturing; or
culturing a host that has introduced therein a gene encoding an enzyme having enzyme activity of hydroxylating a 3-position of a phenol ring of tyrosine but being free of L-DOPA oxidase activity, and that has enzyme activity of having DOPA 4,5-dioxygenase activity, enzyme activity of having amine synthesis activity, and an ability to produce tyrosine or 3-hydroxy-L-tyrosine, and extracting a betaxanthin from the host after the culturing,
wherein the gene encoding an enzyme having enzyme activity of hydroxylating a 3-position of a phenol ring of tyrosine but being free of L-DOPA oxidase activity is any one or more selected from the following:
(1) a gene encoding a polypeptide formed of an amino acid sequence set forth in SEQ ID NO: 51, 53, or 55;
(2) a gene encoding a polypeptide that has 1 to 20 amino acids substituted, deleted, inserted, and/or added in the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(3) a gene encoding a polypeptide that has 90% or more homology to the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(4) a gene formed of DNA formed of a base sequence set forth in SEQ ID NO: 50, 52, or 54;
(5) a gene formed of DNA that hybridizes with DNA formed of a base sequence complementary to DNA formed of the base sequence set forth in SEQ ID NO: 50, 52, or 54 under stringent conditions, and that encodes a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity;
(6) a gene formed of DNA having a 1- to 50-base sequence substituted, deleted, inserted, and/or added in DNA formed of the base sequence set forth in SEQ ID NO: 50, 52, or 54;
(7) a gene formed of DNA having 90% or more homology to DNA formed of the base sequence set forth in SEQ ID NO: 50, 52, or 54; and
(8) a gene formed of DNA formed of a degenerate isomer of the base sequence set forth in SEQ ID NO: 50, 52, or 54.

12. The method of synthesizing a betaxanthin according to the above-mentioned item 11, wherein the gene encoding an enzyme having enzyme activity of hydroxylating a 3-position of a phenol ring of tyrosine but being free of L-DOPA oxidase activity is any one or more selected from SEQ ID NOS: 50, 52, and 54.

13. A betalamic acid synthesis composition for betaxanthin synthesis, including a gene shown in any one of the following items or a vector carrying the gene:
(1) a gene encoding a polypeptide formed of an amino acid sequence set forth in SEQ ID NO: 51, 53, or 55;
(2) a gene encoding a polypeptide that has 1 to 20 amino acids substituted, deleted, inserted, and/or added in the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase
(3) a gene encoding a polypeptide that has 90% or more homology to the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(4) a gene formed of DNA formed of a base sequence set forth in SEQ ID NO: 50, 52, or 54;
(5) a gene formed of DNA that hybridizes with DNA formed of a base sequence complementary to DNA formed of the base sequence set forth in SEQ ID NO: 50, 52, or 54 under stringent conditions, and that encodes a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity;
(6) a gene formed of DNA having a 1- to 50-base sequence substituted, deleted, inserted, and/or added in DNA formed of the base sequence set forth in SEQ ID NO: 50, 52, or 54;
(7) a gene formed of DNA having 90% or more homology to DNA formed of the base sequence set forth in SEQ ID NO: 50, 52, or 54; and
(8) a gene formed of DNA formed of a degenerate isomer of the base sequence set forth in SEQ ID NO: 50, 52, or 53.

14. A betalamic acid synthesis composition for betaxanthin synthesis, including a peptide represented by any one of the following amino acid sequences:
(1) an amino acid sequence set forth in SEQ ID NO: 51, 53, or 55;
(2) an amino acid sequence that has 1 to 20 amino acids substituted, deleted, inserted, and/or added in the amino acid sequence set forth in SEQ ID NO: 51, 53, or 55, and that forms a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity; and
(3) an amino acid sequence that has 90% or more homology to the amino acid sequence set forth in SEQ ID NO: 51, 53, or 55, and that forms a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity.

15. A betaxanthin-producing host having introduced therein the synthesis composition of the above-mentioned item 13 or 14.

16. A betaxanthin-producing host,
the host having introduced therein a gene encoding an enzyme having enzyme activity of hydroxylating a 3-position of a phenol ring of tyrosine but being free of L-DOPA oxidase activity, and a gene encoding an enzyme having DOPA 4,5-dioxygenase activity, and having an ability to produce tyrosine or 3-hydroxy-L-tyrosine, or
the host having introduced therein a gene encoding an enzyme having enzyme activity of hydroxylating a 3-position of a phenol ring of tyrosine but being free of L-DOPA oxidase activity, and having enzyme activity of having DOPA 4,5-dioxygenase activity, and an ability to produce tyrosine or 3-hydroxy-L-tyrosine,
wherein the gene encoding an enzyme having enzyme activity of hydroxylating a 3-position of a phenol ring of tyrosine but being free of L-DOPA oxidase activity is any one or more selected from the following:
(1) a gene encoding a polypeptide formed of an amino acid sequence set forth in SEQ ID NO: 51, 53, or 55;
(2) a gene encoding a polypeptide that has 1 to 20 amino acids substituted, deleted, inserted, and/or added in the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase
(3) a gene encoding a polypeptide that has 90% or more homology to the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(4) a gene formed of DNA formed of a base sequence set forth in SEQ ID NO: 50, 52, or 54;
(5) a gene formed of DNA that hybridizes with DNA formed of a base sequence complementary to DNA formed of the base sequence set forth in SEQ ID NO: 50, 52, or 54 under stringent conditions, and that encodes a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity;
(6) a gene formed of DNA having a 1- to 50-base sequence substituted, deleted, inserted, and/or added in DNA formed of the base sequence set forth in SEQ ID NO: 50, 52, or 54;
(7) a gene formed of DNA having 90% or more homology to DNA formed of the base sequence set forth in SEQ ID NO: 50, 52, or 54; and
(8) a gene formed of DNA formed of a degenerate isomer of the base sequence set forth in SEQ ID NO: 50, 52, or 54.

17. An amyloid-β polymerization inhibitor or a therapeutic or preventive agent for Alzheimer's, including any one of the following:
(1) celosianin II;
(2) celosianin II obtained by the method of synthesizing celosianin II of any one of the above-mentioned items 1 to 5 and 9;
(3) celosianin II obtained from the celosianin II-producing host of the above-mentioned item 8 or 10;
(4) amaranthin;
(5) betanin;
(6) ferulic acid;
(7) a betaxanthin;
(8) a betaxanthin obtained by the method of synthesizing a betaxanthin of the above- mentioned item 11 or 12; and
(9) a betaxanthin obtained from the betaxanthin-producing host of the above-mentioned item 16.

18. An amyloid peptide aggregation inhibitor, including any one of the following:
(1) celosianin II;
(2) celosianin II obtained by the method of synthesizing celosianin II of any one of the above-mentioned items 1 to 5 and 9;
(3) celosianin II obtained from the celosianin II-producing host of the above-mentioned item 8 or 10;
(4) amaranthin;
(5) betanin;
(6) ferulic acid; and
(7) a betaxanthin.

19. A preventive and/or therapeutic agent for a neurodegenerative disease, Alzheimer's disease, systemic amyloidosis, Parkinson's disease, Huntington's disease, a prion disease, multiple sclerosis, or amyotrophic lateral sclerosis, the preventive and/or therapeutic agent including the amyloid peptide aggregation inhibitor of the above-mentioned item 18 as an active ingredient.

20. An HIV-1 protease activity inhibitor, including any one of the following:
(1) celosianin II;
(2) celosianin II obtained by the method of synthesizing celosianin II of any one of the above-mentioned items 1 to 5 and 9; and
(3) celosianin II obtained from the celosianin II-producing host of the above-mentioned item 8 or 10.

### Advantageous Effects of Invention

According to the present invention, the method of synthesizing celosianin II, the method of synthesizing a betaxanthin, the amyloid-β polymerization inhibitor or the therapeutic or preventive agent for Alzheimer's, the amyloid peptide aggregation inhibitor, and the HIV-1 protease activity inhibitor can be provided.

### BRIEF DESCRIPTION OF DRAWINGS

FIG. 1 is an illustration of the structures of betalain pigments.

FIG. 2 shows the accumulation of betalain pigments in hypocotyls.

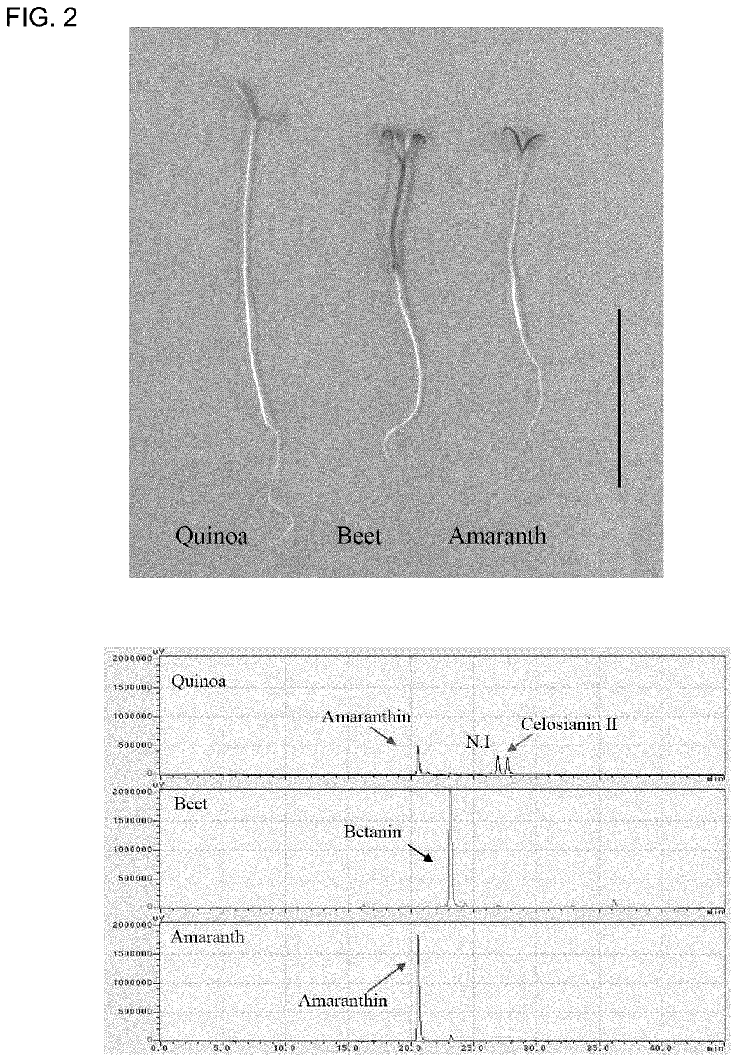

FIG. 3 shows the results of evaluation of the stabilities of betalain pigments.

FIG. 4 shows the results of evaluation of inhibitory activity on HIV-1 protease activity.

FIG. 5 shows the results of evaluation of the polymerization inhibitory activities of betalain pigments on amyloid-β.

FIG. 6 is an overview of the results of a search for a celosianin II synthetase gene.

FIG. 7 is a scheme for a biosynthesis pathway of betalain pigments.

FIG. 8 is a schematic diagram of celosianin II biosynthesis.

FIG. 9 is a scheme for a biosynthesis pathway of a betaxanthin.

FIG. 10 shows the results of activity evaluation in tobacco BY2 cells.

FIG. 11 shows the results of evaluation of the polymerization inhibitory activity of a betaxanthin on amyloid-β.

FIGS. 12A-H show the results of observation of amyloid peptide aggregation inhibition using an electron microscope. A and B: 25 μM Aβ40+water, C and D: 25 μM Aβ40+50 μM betanin, E: 25 μM Aβ40+50 μM amaranthin, F: 25 μM Aβ40+50 μM celosianin II, G: 25 μM Aβ40+50 μM ferulic acid, H: 25 μM Aβ40+50 μM betaxanthin. Scale bars: 200 μm.

FIG. 13A shows the ratio of nematodes that are not paralyzed in a group of nematodes fed with dextrin (control), and FIG. 13B shows the ratio of nematodes that are not paralyzed in a group of nematodes fed with betanin. Vertical axis: ratio of nematodes that are not paralyzed, horizontal axis: number of days of adulthood. FIG. 13C shows nematodes grown with dextrin-mixed food, and FIG. 13D shows normal nematodes.

FIG. 14 shows results for an amaranth plant body caused to express XM_021915966 in celosianin II synthesis activity evaluation. The gene XM_021915966 was transiently expressed, and pigments were analyzed by HPLC. Infected plant body: amaranth (which does not accumulate celosianin II). In the amaranth plant body caused to express XM_021915966, the accumulation of celosianin II was able to be newly found.

FIG. 15 shows results for quinoa caused to transiently express XM_021915966 in celosianin II synthesis activity evaluation. The candidate gene was transiently expressed, and pigments were analyzed by HPLC. Infected plant body: quinoa. In the quinoa caused to transiently express XM_021915966, the accumulation of celosianin II was increased about 2-fold.

### DESCRIPTION OF EMBODIMENTS

The present invention relates to a method of synthesizing celosianin II, a celosianin II synthesis composition, and a celosianin II-producing host.

The present invention relates to a method of synthesizing a betaxanthin, a betalamic acid synthesis composition for betaxanthin synthesis, and a betaxanthin-producing host.

### Betalain Pigment

Betalain pigments in the present invention are classified into betaxanthins and betacyanins on the basis of their structural features. The betaxanthins exhibit yellow colors and the betacyanins exhibit red-violet colors, and hence the betaxanthins and the betacyanins have heretofore been utilized as natural colorants. The term “betacyanins” collectively refers to a group of compounds in each of which a sugar is glycosidically bonded to a phenolic hydroxy group of betanidin.

### Celosianin II Synthesis System

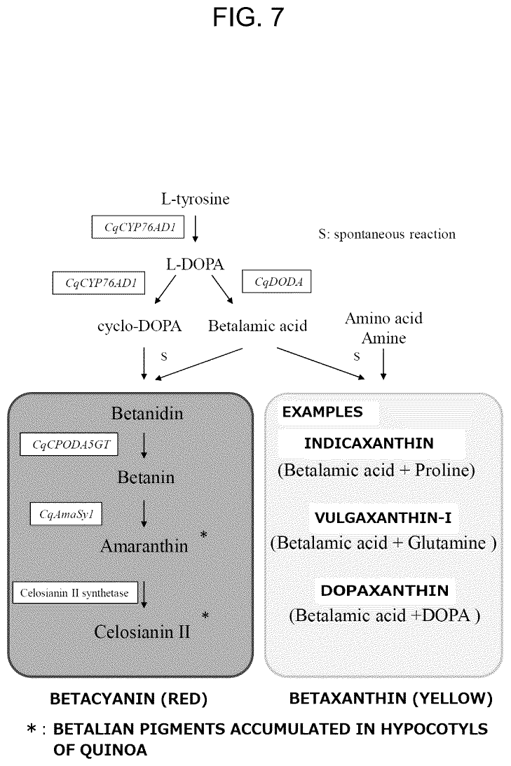

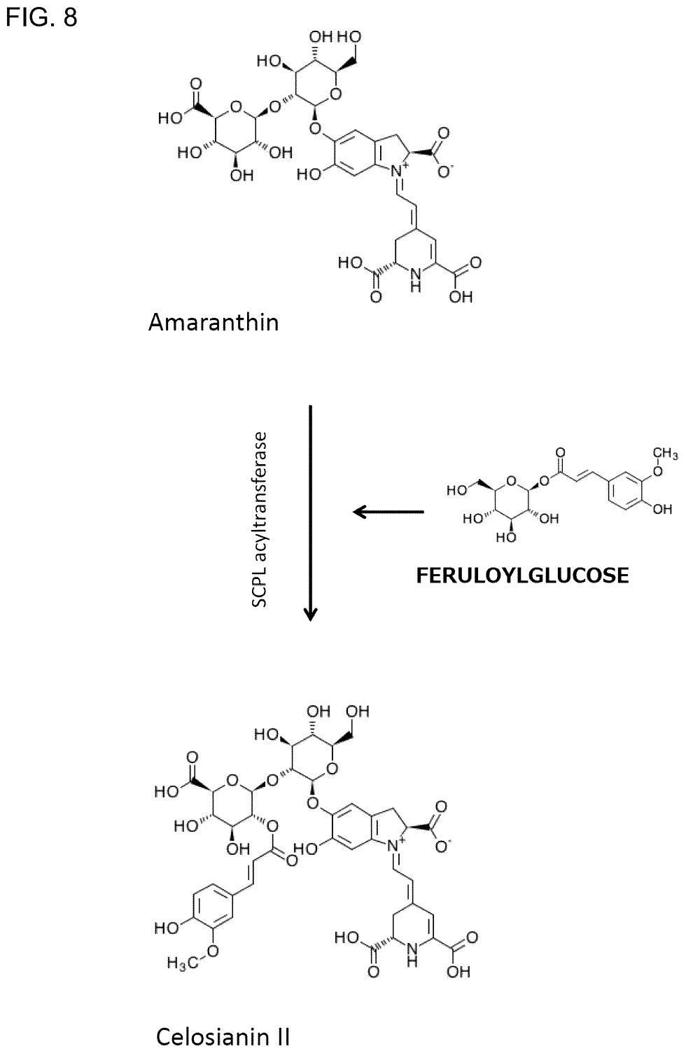

Schemes for betalain pigment biosynthetic pathways in the present invention are illustrated in FIG. 7 and FIG. 8.

As apparent from the illustrations in FIG. 7 and FIG. 8, when a host having an ability to produce tyrosine (L-tyrosine) or 3-hydroxy-L-tyrosine (L-DOPA), and an amino acid or an amine (e.g., putrescine, spermidine, or spermine) (including a host capable of incorporating these compounds from outside) has the following enzyme activities, celosianin II can be synthesized.

Enzyme (e.g., CqCYP76AD1) activity of hydroxylating the 3-position of the phenol ring of tyrosine, L-DOPA oxidase (e.g., CqCYP76AD1) activity and/or DOPA 4,5-dioxygenase (e.g., CqDODA-1) activity, the activity of an enzyme having activity of glycosylating a phenolic hydroxy group {e.g., an enzyme having betanidin 5-O-glucosyltransferase (5GT) activity (e.g., Cyclo-DOPA 5-O-glucosyltransferase, CqCDOPA5GT)}, enzyme (e.g., CqAmaSyl1) activity of having activity of adding glucuronic acid, and celosianin II synthetase activity of the present invention.

CqCYP76AD1 has both of the following properties: enzyme activity of hydroxylating the 3-position of the phenol ring of tyrosine; and L-DOPA oxidase activity.

According to the results of Examples to be described below, a plant body that is a host having introduced therein a gene group of a gene encoding an enzyme (CqCYP76AD1-1) having activity of hydroxylating the 3-position of the phenol ring of tyrosine, a gene encoding an enzyme having L-DOPA oxidase activity and/or a gene encoding an enzyme (CqDODA-1) having DOPA 4,5-dioxygenase activity, a gene encoding an enzyme (CqCDOPA5GT) having activity of glycosylating a phenolic hydroxy group, a gene encoding an enzyme (CqAmaSy1) having activity of adding glucuronic acid, and a gene encoding the celosianin II synthetase of the present invention has an ability to synthesize celosianin II, which is a betalain pigment.

As apparent from the illustrations in FIG. 7 and FIG. 8, the present invention also encompasses the following celosianin II synthesis method.
(1) Amaranthin is brought into contact with the celosianin II synthetase of the present invention. As required, a metal ion is added.
(2) Betanin is brought into contact with an enzyme having activity of adding glucuronic acid to provide amaranthin, which is then brought into contact with the celosianin II synthetase of the present invention. As required, a metal ion is added.
(3) Betanidin is brought into contact with an enzyme having activity of glycosylating a phenolic hydroxy group to provide betanin, which is then brought into contact with an enzyme having activity of adding glucuronic acid to provide amaranthin, which is then brought into contact with the celosianin II synthetase of the present invention. As required, a metal ion is added.

### Enzyme for Hydroxylating 3-Position of Phenol Ring of Tyrosine

The enzyme for hydroxylating the 3-position of the phenol ring of tyrosine in the present invention has activity of allowing a hydroxy group to be added to the 3-position of the phenol ring that tyrosine has, and the enzyme may be derived from any species as long as the enzyme has the activity.

Examples of the enzyme for hydroxylating the 3-position of the phenol ring of tyrosine include tyrosinase, cytochromes P450 (in particular, CYP76AD1, CYP76AD2, CYP76AD3, and the like), and catechol oxidase. Of those, CqCYP76AD1 (base sequence: SEQ ID NO: 17, amino acid sequence: SEQ ID NO: 18, accession number: XP_021769302) is preferred.

### Enzyme Having L-DOPA Oxidase Activity

The enzyme having L-DOPA oxidase activity in the present invention has activity of converting L-DOPA into cyclo-DOPA, and may be derived from any species as long as the enzyme has the activity.

Examples of the enzyme having L-DOPA oxidase activity include tyrosinase and cytochromes P450, including a subfamily a of a CYP76AD group, such as CYP76AD1, CYP76AD2, and CYP76AD3. Of those, CqCYP76AD1 (base sequence: SEQ ID NO: 17, amino acid sequence: SEQ ID NO: 18) is preferred.

### Enzyme having Activity of Glycosylating Phenolic Hydroxy Group

The enzyme having activity of glycosylating a phenolic hydroxy group in the present invention has an ability to synthesize betanin through enzyme activity of having activity of glycosylating a phenolic hydroxy group, i.e., activity of glycosylating a phenolic hydroxy group present at the 5-position or the 6-position of a cyclo-DOPA skeleton, and may be derived from any species as long as the enzyme has the activity.

The enzyme having activity of glycosylating a phenolic hydroxy group encompasses a betanidin-to-betanin synthetase.

Examples of the enzyme having activity of glycosylating a phenolic hydroxy group include cyclo-DOPA 5-O-glucosyltransferase (CDOPASGT), betanidin 5-O-glucosyltransferase (B5GT), and betanidin 6-O-glucosyltransferase. Of those, CqCDOPA5GT (base sequence: SEQ ID NO: 19, amino acid sequence: SEQ ID NO: 20, accession number: XP_021748306) is preferred. (Betanidin-to-Gomphrenin-I Synthetase)

The betanidin-to-gomphrenin-I synthetase in the present invention is not particularly limited as long as gomphrenin-I can be synthesized from betanidin, and examples thereof may include betanidin 6-O-glucosyltransferase (6GT (B6GT): SEQ ID NOS: 21 and 22) and cyclo-DOPA 6-O-glucosyltransferase (CDOPA6GT) (see: Substrate specificity and sequence analysis define a polyphyletic origin of betanidin 5-and 6-O-glucosyltransferase from Dorotheanthus bellidiformis. planta 214, 492-495).

### Enzyme Having Activity of Adding Glucuronic Acid

The enzyme having activity of adding glucuronic acid in the present invention has an ability to synthesize amaranthin through enzyme activity of having activity of adding glucuronic acid, and may be derived from any species as long as the enzyme has the activity.

The enzyme having activity of adding glucuronic acid encompasses, for example, a betanin-to-amaranthin synthetase, and an enzyme having activity of synthesizing amaranthin by bonding glucuronic acid to betanin (e.g., activity of synthesizing UDP-glucoronate (betanin beta-D-glucuronosyltransferase)).

Examples of the gene encoding the enzyme having activity of adding glucuronic acid (amaranthin synthetase gene) may include the following:
(1) a gene encoding a polypeptide formed of an amino acid sequence set forth in SEQ ID NO: 24, 26, 28, 30, 32, or 34;
(2) a gene encoding a polypeptide that has 1 to 20 amino acids substituted, deleted, inserted, and/or added in the amino acid sequence set forth in SEQ ID NO: 24, 26, 28, 30, 32, or 34, and that has a substantially equivalent ability to synthesize amaranthin to that of the amino acid sequence set forth in SEQ ID NO: 24, 26, 28, 30, 32, or 34;
(3) a gene encoding a polypeptide that has 90% or more homology to the amino acid sequence set forth in SEQ ID NO: 24, 26, 28, 30, 32, or 34, and that has a substantially equivalent ability to synthesize amaranthin to that of the amino acid sequence set forth in SEQ ID NO: 24, 26, 28, 30, 32, or 34;
(4) a gene formed of DNA formed of a base sequence set forth in SEQ ID NO: 23, 25, 27, 29, 31, or 33;
(5) a gene formed of DNA that hybridizes with DNA formed of a base sequence complementary to DNA formed of the base sequence set forth in SEQ ID NO: 23, 25, 27, 29, 31, or 33 under stringent conditions, and that encodes a polypeptide having an ability to synthesize amaranthin;
(6) a gene formed of DNA having a 1- to 50-base sequence substituted, deleted, inserted, and/or added in DNA formed of the base sequence set forth in SEQ ID NO: 23, 25, 27, 29, 31, or
(7) a gene formed of DNA having 90% or more homology to DNA formed of the base sequence set forth in SEQ ID NO: 23, 25, 27, 29, 31, or 33; and
(8) a gene formed of DNA formed of a degenerate isomer of the base sequence set forth in SEQ ID NO: 23, 25, 27, 29, 31, or 33.

The gene of the above-mentioned item (2) is a gene encoding a polypeptide having introduced therein such a mutation as not to cause the loss of the ability to synthesize amaranthin. Such mutation encompasses an artificial mutation as well as a naturally occurring mutation. As means for causing the artificial mutation, there may be given, for example, a site-directed mutagenesis method (Nucleic Acids Res. 10, 6487-6500, 1982). The number of mutated amino acids is generally 20 or less, preferably 10 or less, more preferably 5 or less, most preferably 3. Whether or not the polypeptide having introduced therein the mutation retains the ability to synthesize amaranthin can be found by, for example, introducing a gene encoding the polypeptide having introduced therein the mutation into a plant body or the like, and checking the ability to synthesize amaranthin in the plant body.

With regard to the gene of the above-mentioned item (3), the “substantially equivalent ability to synthesize amaranthin to that of the amino acid sequence set forth in SEQ ID NO: 24, 26, 28, 30, 32, or 34” may be stronger or weaker in degree of the action as compared to the substantially equivalent ability to synthesize amaranthin to that of the amino acid sequence set forth in SEQ ID NO: 24, 26, 28, 30, 32, or 34. The degree of the action may be, for example, about 50%, about 60%, about 70%, about 80%, about 90%, about 100%, about 110%, about 120%, about 130%, about 140%, or about 150% as compared to the ability of the amino acid sequence set forth in SEQ ID NO: 24, 26, 28, 30, 32, or 34 to synthesize amaranthin.

In addition, the identity may be calculated using the Basic Local Alignment Search Tool at the National Center for Biological Information (BLAST) or the like (using, for example, default, namely initially set, parameters).

The gene of the above-mentioned item (5) is a gene obtained by utilizing hybridization between DNAs. The term “stringent conditions” in this gene refers to conditions under which only specific hybridization occurs and non-specific hybridization does not occur. Such conditions are generally conditions such as hybridization in a buffer containing 5×SSC and 1% SDS at 37° C. and washing treatment with a buffer containing 1×SSC and 0.1% SDS at 37° C., preferably conditions such as hybridization in a buffer containing 5×SSC and 1% SDS at 42° C. and washing treatment with a buffer containing 0.5×SSC and 0.1% SDS at 42° C., more preferably conditions such as hybridization in a buffer containing 5×SSC and 1% SDS at 65° C. and washing treatment with a buffer containing 0.2×SSC and 0.1% SDS at 65° C. Whether or not DNA obtained by utilizing hybridization encodes a polypeptide having activity can be found by, for example, introducing the DNA into a plant body or the like, and checking the ability of the plant body to synthesize amaranthin. The DNA obtained by hybridization generally has high identity to the gene of the above-mentioned item (4) (SEQ ID NO: 23, 25, 27, 29, 31, or 33). The “high identity” refers to 90% or more identity, preferably 95% or more identity, more preferably 98% or more identity.

The gene of the above-mentioned item (6) is a gene formed of DNA having a 1- to 50-base sequence, preferably a 1- to 30-base sequence, more preferably a 1- to 20-base sequence, most preferably a 1- to 10-base sequence, even most preferably a 1- to 5-base sequence substituted, deleted, inserted, and/or added in DNA formed of the base sequence set forth in SEQ ID NO: 23, 25, 27, 29, 31, or 33.

The gene of the above-mentioned item (7) is a gene formed of DNA having 90% or more, preferably 93% or more, more preferably 95% or more, most preferably 98% or more identity to DNA formed of the base sequence set forth in SEQ ID NO: 23, 25, 27, 29, 31, or 33.

The enzyme having enzyme activity of having activity of adding glucuronic acid in the present invention has any one or more amino acid sequences selected from the following:
(1) an amino acid sequence set forth in SEQ ID NO: 24, 26, 28, 30, 32, or 34;
(2) an amino acid sequence that has 1 to 20 amino acids substituted, deleted, inserted, and/or added in the amino acid sequence set forth in SEQ ID NO: 24, 26, 28, 30, 32, or 34, and that forms a polypeptide having a substantially equivalent ability to synthesize amaranthin to that of the amino acid sequence set forth in SEQ ID NO: 57, 59, 61, 63, 65, 67, 69, 2, 4, 6, 8, 10, 12, 14, or 16; and
(3) an amino acid sequence that has 90% or more homology to the amino acid sequence set forth in SEQ ID NO: 24, 26, 28, 30, 32, or 34, and that forms a polypeptide having a substantially equivalent ability to synthesize amaranthin to that of the amino acid sequence set forth in SEQ ID NO: 24, 26, 28, 30, 32, or 34.

In the introduction of a mutation into a peptide, for example, a substitution between homologous amino acids (e.g., polar amino acids, non-polar amino acids, hydrophobic amino acids, hydrophilic amino acids, positively charged amino acids, negatively charged amino acids, and aromatic amino acids) is easily conceivable from the viewpoint of preventing basic properties (e.g., physical properties, function, physiological activity, or immunological activity) of the peptide from being changed.

### Enzyme Having DOPA 4,5-Dioxygenase Activity

The enzyme having DOPA 4,5-dioxygenase activity in the present invention has an ability to convert L-DOPA into 4,5-seco-DOPA through activity of catalyzing extradiol cleavage of L-3,4-dihydroxyphenylalanine. Further, 4,5-seco-DOPA converts into betalamic acid through a spontaneous reaction.

The gene encoding the enzyme having DOPA 4,5-dioxygenase activity in the present invention is any one or more selected from the following:
(1) a gene encoding a polypeptide formed of an amino acid sequence set forth in SEQ ID NO: 35 (accession number: XP_021769303), 37 (accession number: XP_021769301), 39, 41, 43, 45, or 47;
(2) a gene encoding a polypeptide that has 1 to 20 amino acids substituted, deleted, inserted, and/or added in the amino acid sequence set forth in SEQ ID NO: 35, 37, 39, 41, 43, 45, or 47, and that has a substantially equivalent ability to convert L-DOPA into 4,5-seco-DOPA to that of the amino acid sequence set forth in SEQ ID NO: 35, 37, 39, 41, 43, or 45;
(3) a gene encoding a polypeptide that has 90% or more homology to the amino acid sequence set forth in SEQ ID NO: 35, 37, 39, 41, 43, 45, or 47, and that has a substantially equivalent ability to convert L-DOPA into 4,5-seco-DOPA to that of the amino acid sequence set forth in SEQ ID NO: 35, 37, 39, 41, 43, 45, or 47;
(4) a gene formed of DNA formed of a base sequence set forth in SEQ ID NO: 36, 38, 40, 42, 44, 46, or 48;
(5) a gene formed of DNA that hybridizes with DNA formed of a base sequence complementary to DNA formed of the base sequence set forth in SEQ ID NO: 36, 38, 40, 42, 44, 46, or 48 under stringent conditions, and that encodes a polypeptide having an ability to convert L-DOPA into 4,5-seco-DOPA;
(6) a gene formed of DNA having a 1- to 50-base sequence substituted, deleted, inserted, and/or added in DNA formed of the base sequence set forth in SEQ ID NO: 36, 38, 40, 42, 44, 46, or 48;
(7) a gene formed of DNA having 90% or more homology to DNA formed of the base sequence set forth in SEQ ID NO: 36, 38, 40, 42, 44, 46, or 48;
(8) the gene of any one or more of the above-mentioned items (1) to (7) including a gene encoding a polypeptide formed of an amino acid sequence set forth in SEQ ID NO: 33; and
(9) a gene formed of DNA formed of a degenerate isomer of the base sequence set forth in SEQ ID NO: 36, 38, 40, 42, 44, 46, or 48.

The gene of the above-mentioned item (2) is a gene encoding a polypeptide having introduced therein such a mutation as not to cause the loss of the ability to convert L-DOPA into 4,5-seco-DOPA. Such mutation may be introduced by the method described above.

With regard to the gene of the above-mentioned item (3), the “substantially equivalent ability to convert L-DOPA into 4,5-seco-DOPA to that of the amino acid sequence set forth in SEQ ID NO: 35, 37, 39, 41, 43, 45, or 47” may be stronger or weaker in degree of the action as compared to the ability of the amino acid sequence set forth in SEQ ID NO: 35, 37, 39, 41, 43, 45, or 47 to convert L-DOPA into 4,5-seco-DOPA. The degree of the action may be, for example, about 50%, about 60%, about 70%, about 80%, about 90%, about 100%, about 110%, about 120%, about 130%, about 140%, or about 150% as compared to the ability of the amino acid sequence set forth in SEQ ID NO: 35, 37, 39, 41, 43, 45, or 47 to convert L-DOPA into 4,5-seco-DOPA.

The gene of the above-mentioned item (5) is a gene obtained by utilizing hybridization between DNAs. DNA obtained by hybridization generally has high identity to the gene of the above-mentioned item (4) (SEQ ID NO: 18, 20, 22, 24, 26, 28, or 48). The “high identity” refers to 90% or more identity, preferably 95% or more identity, more preferably 98% or more identity.

The gene of the above-mentioned item (6) is a gene formed of DNA having a 1- to 50-base sequence, preferably a 1- to 30-base sequence, more preferably a 1- to 20-base sequence, most preferably a 1- to 10-base sequence, even most preferably a 1- to 5-base sequence substituted, deleted, inserted, and/or added in DNA formed of the base sequence set forth in SEQ ID NO: 36, 38, 40, 42, 44, 46, or 48.

The gene of the above-mentioned item (7) is a gene formed of DNA having 90% or more, preferably 93% or more, more preferably 95% or more, most preferably 98% or more identity to DNA formed of the base sequence set forth in SEQ ID NO: 36, 38, 40, 42, 44, 46, or 48.

The enzyme having DOPA 4,5-dioxygenase activity in the present invention is any one or more selected from the following:
(1) an amino acid sequence set forth in SEQ ID NO: 35, 37, 39, 41, 43, 45, or 47;
(2) an amino acid sequence that has 1 to 20 amino acids substituted, deleted, inserted, and/or added in the amino acid sequence set forth in SEQ ID NO: 35, 37, 39, 41, 43, 45, or 47, and that has a substantially equivalent ability to convert L-DOPA into 4,5-seco-DOPA to that of the amino acid sequence set forth in SEQ ID NO: 35, 37, 39, 41, 43, 45, or 47;
(3) an amino acid sequence that has 90% or more homology to the amino acid sequence set forth in SEQ ID NO: 35, 37, 39, 41, 43, 45, or 47, and that has a substantially equivalent ability to convert L-DOPA into 4,5-seco-DOPA to that of an amino acid sequence set forth in SEQ ID NO: 57, 59, 61, 63, 65, 67, 69, or 2; and
(4) the amino acid sequence of any one or more of the above-mentioned items (1) to (3) including an amino acid sequence set forth in SEQ ID NO: 33.

In the introduction of a mutation into a peptide, for example, a substitution between homologous amino acids (e.g., polar amino acids, non-polar amino acids, hydrophobic amino acids, hydrophilic amino acids, positively charged amino acids, negatively charged amino acids, and aromatic amino acids) is easily conceivable from the viewpoint of preventing basic properties (e.g., physical properties, function, physiological activity, or immunological activity) of the peptide from being changed.

In particular, an amino acid sequence set forth in SEQ ID NO: 49 contains the sequence of an active site. When a mutation is introduced into this sequence, the ability to convert L-DOPA into 4,5-seco-DOPA is highly liable to be lost, and hence it is preferred that the mutation be introduced at an amino acid outside those domains.

### Celosianin II Synthetase

The celosianin II synthetase in the present invention is not particularly limited as long as the synthetase has an ability to synthesize celosianin II through activity of synthesizing celosianin II from amaranthin. The celosianin II synthetase has activity of adding an aromatic carboxylic acid other than ferulic acid (e.g., caffeic acid or coumaric acid) to amaranthin as well as synthesizing celosianin II by adding ferulic acid to amaranthin. Further, the celosianin II synthetase has activity of adding an aromatic carboxylic acid to a betacyanin other than amaranthin (e.g., betanin or gomphrenin) as well.

Examples of the gene encoding the celosianin II synthetase (celosianin II synthetase gene) may include the following:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

The gene of the above-mentioned item (2) is a gene encoding a polypeptide having introduced therein such a mutation as not to cause the loss of the ability to synthesize celosianin II. Such mutation encompasses an artificial mutation as well as a naturally occurring mutation.

As means for causing the artificial mutation, there may be given, for example, a site-directed mutagenesis method (Nucleic Acids Res. 10, 6487-6500, 1982). The number of mutated amino acids is generally 20 or less, preferably 10 or less, more preferably 5 or less, most preferably 3. Whether or not the polypeptide having introduced therein the mutation retains the ability to synthesize celosianin II can be found by, for example, introducing a gene encoding the polypeptide having introduced therein the mutation into a plant body or the like, and checking the ability to synthesize celosianin II in the plant body.

With regard to the gene of the above-mentioned item (3), the “substantially equivalent ability to synthesize celosianin II to that of the amino acid sequence set forth in SEQ ID NO: 57, 59, 61, 63, 65, 67, 69, 2, 4, 6, 8, 10, 12, 14, or 16” may be stronger or weaker in degree of the action as compared to the substantially equivalent ability to synthesize celosianin II to that of the amino acid sequence set forth in SEQ ID NO: 57, 59, 61, 63, 65, 67, 69, 2, 4, 6, 8, 10, 12, 14, or 16. The degree of the action may be, for example, about 50%, about 60%, about 70%, about 80%, about 90%, about 100%, about 110%, about 120%, about 130%, about 140%, or about 150% as compared to the ability of the amino acid sequence set forth in SEQ ID NO: 57, 59, 61, 63, 65, 67, 69, 2, 4, 6, 8, 10, 12, 14, or 16 to synthesize celosianin II.

In addition, the identity may be calculated using the Basic Local Alignment Search Tool at the National Center for Biological Information (BLAST) or the like (using, for example, default, namely initially set, parameters).

The gene of the above-mentioned item (5) is a gene obtained by utilizing hybridization between DNAs. The term “stringent conditions” in this gene refers to conditions under which only specific hybridization occurs and non-specific hybridization does not occur. Such conditions are generally conditions such as hybridization in a buffer containing 5×SSC and 1% SDS at 37° C. and washing treatment with a buffer containing 1×SSC and 0.1% SDS at 37° C., preferably conditions such as hybridization in a buffer containing 5×SSC and 1% SDS at 42° C. and washing treatment with a buffer containing 0.5×SSC and 0.1% SDS at 42° C., more preferably conditions such as hybridization in a buffer containing 5×SSC and 1% SDS at 65° C. and washing treatment with a buffer containing 0.2×SSC and 0.1% SDS at 65° C. Whether or not DNA obtained by utilizing hybridization encodes a polypeptide having activity can be found by, for example, introducing the DNA into a plant body or the like, and checking the ability of the plant body to synthesize celosianin II. The DNA obtained by hybridization generally has high identity to the gene of the above-mentioned item (4) (SEQ ID NO: 56, 58, 60, 62, 64, 66, 68, 1, 3, 5, 7, 9, 11, 13, or 15). The “high identity” refers to 90% or more identity, preferably 95% or more identity, more preferably 98% or more identity.

The gene of the above-mentioned item (6) is a gene formed of DNA having a 1- to 50-base sequence, preferably a 1- to 30-base sequence, more preferably a 1- to 20-base sequence, most preferably a 1- to 10-base sequence, even most preferably a 1- to 5-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15.

The gene of the above-mentioned item (7) is a gene formed of DNA having 90% or more, preferably 93% or more, more preferably 95% or more, most preferably 98% or more identity to DNA formed of the base sequence set forth in SEQ ID NO: 56, 58, 60, 62, 64, 66, 68, 1, 3, 5, 7, 9, 11, 13, or 15.

The enzyme having celosianin II synthetase activity in the present invention has any one or more amino acid sequences selected from the following:
(1) an amino acid sequence set forth in SEQ ID NO: 57, 59, 61, 63, 65, 67, 69, 2, 4, 6, 8, 10, 12, 14, or 16;
(2) an amino acid sequence that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that forms a polypeptide having a substantially equivalent ability to synthesize celosianin II to that of the amino acid sequence set forth in SEQ ID NO: 57, 59, 61, 63, 65, 67, 69, 2, 4, 6, 8, 10, 12, 14, or 16; and
(3) an amino acid sequence that has 90% or more homology to the amino acid sequence set forth in SEQ ID NO: 57, 59, 61, 63, 65, 67, 69, 2, 4, 6, 8, 10, 12, 14, or 16, and that forms a polypeptide having a substantially equivalent ability to synthesize celosianin II to that of the amino acid sequence set forth in SEQ ID NO: 57, 59, 61, 63, 65, 67, 69, 2, 4, 6, 8, 10, 12, 14, or 16.

In the introduction of a mutation into a peptide, for example, a substitution between homologous amino acids (e.g., polar amino acids, non-polar amino acids, hydrophobic amino acids, hydrophilic amino acids, positively charged amino acids, negatively charged amino acids, and aromatic amino acids) is easily conceivable from the viewpoint of preventing basic properties (e.g., physical properties, function, physiological activity, or immunological activity) of the peptide from being changed.

### Method of Synthesizing Celosianin II

A method of synthesizing celosianin II of the present invention may be exemplified by the following:
(1) culturing a host that has introduced therein a gene encoding a celosianin II synthetase, a gene encoding an enzyme having activity of hydroxylating a 3-position of a phenol ring of tyrosine, a gene encoding an enzyme having L-DOPA oxidase activity and/or a gene encoding an enzyme having DOPA 4,5-dioxygenase activity, a gene encoding an enzyme having activity of glycosylating a phenolic hydroxy group, and a gene encoding an enzyme having activity of adding glucuronic acid, and that has an ability to produce tyrosine or L-DOPA, and extracting celosianin II from the host after the culturing; or
(2) culturing a host that has introduced therein a gene encoding a celosianin II synthetase, and that has enzyme activity of having activity of hydroxylating a 3-position of a phenol ring of tyrosine, L-DOPA oxidase activity and/or DOPA 4,5-dioxygenase activity, enzyme activity of having activity of glycosylating a phenolic hydroxy group, enzyme activity of having activity of adding glucuronic acid, and an ability to produce tyrosine or 3-hydroxy-L-tyrosine, and extracting celosianin II from the host after the culturing.

### Host

The host to be used in the method of synthesizing celosianin II or a betaxanthin of the present invention is not particularly limited as long as the host has an ability to produce tyrosine or 3-hydroxy-L-tyrosine. For example, there may be used a synthesis system known per se, such as a recombinant Escherichia coli protein synthesis system, an insect protein synthesis system, a yeast protein synthesis system, a plant cell protein synthesis system, a cell-free protein synthesis system, a plant protein synthesis system, or animal cultured cells.

A method known per se may be used as a method of introducing each gene into the host to be used in the method of synthesizing celosianin II or a betaxanthin of the present invention. For example, the gene may be introduced into the host using a vector carrying the gene.

As the vector, a viral vector known per se, in particular, a plant virus vector (e.g., a vector derived from a virus belonging to the genus Tobamovirus, a tobacco mosaic virus vector, or a tomato mosaic virus vector) may be utilized.

In addition, an Agrobacterium method involving using a Ti plasmid may be utilized.

### Method of Introducing Each Gene Into Host

In the method of synthesizing celosianin II or a betaxanthin of the present invention, with regard to the method of introducing each gene into the host, the gene may be introduced into a plant body by a method known per se. For example, each gene may be introduced into the host by applying a solution containing a plant virus vector carrying each gene to a leaf, a stalk, a root, an ear, or the like of the plant body. Other examples of the method may include a particle gun method and an Agrobacterium method.

### Method of Introducing Each Protein Into Host

In the method of synthesizing celosianin II or a betaxanthin of the present invention, with regard to a method of introducing each protein into the host (in particular, a plant body), the protein may be introduced into the plant body by a method known per se. For example, each protein may be introduced into the host by applying a solution containing each protein to a leaf, a stalk, a root, an ear, or the like of the plant body. Other examples of the method may include a particle gun method and an Agrobacterium method.

### Celosianin II-Producing Host

A celosianin II-producing host of the present invention has, for example, any one or more of the following configurations and features:
(1) the host has introduced therein a gene encoding a celosianin II synthetase, a gene encoding an enzyme having activity of hydroxylating a 3-position of a phenol ring of tyrosine, a gene encoding an enzyme having L-DOPA oxidase activity and/or a gene encoding an enzyme having DOPA 4,5-dioxygenase activity, a gene encoding an enzyme having activity of glycosylating a phenolic hydroxy group, and a gene encoding an enzyme having activity of adding glucuronic acid, and has an ability to produce tyrosine or 3-hydroxy-L-tyrosine;
(2) the host has introduced therein a gene encoding a celosianin II synthetase, and has enzyme activity of having activity of hydroxylating a 3-position of a phenol ring of tyrosine, L-DOPA oxidase activity and/or DOPA 4,5-dioxygenase activity, enzyme activity of having activity of glycosylating a phenolic hydroxy group, enzyme activity of having activity of adding glucuronic acid, and an ability to produce tyrosine or 3-hydroxy-L-tyrosine;
(3) the host has introduced therein a gene encoding a celosianin II synthetase, and has an ability to produce amaranthin;
(4) the host has introduced therein a gene encoding a celosianin II synthetase and a gene encoding an enzyme having activity of adding glucuronic acid, and has an ability to produce betanin;
(5) the host has introduced therein a gene encoding a celosianin II synthetase, and has enzyme activity of having activity of adding glucuronic acid and an ability to produce betanin;
(6) the host has introduced therein a gene encoding a celosianin II synthetase, a gene encoding an enzyme having activity of glycosylating a phenolic hydroxy group, and a gene encoding an enzyme having activity of adding glucuronic acid, and has an ability to produce betanidin;
(7) the host has introduced therein a gene encoding a celosianin II synthetase, and has enzyme activity of having activity of glycosylating a phenolic hydroxy group, enzyme activity of having activity of adding glucuronic acid, and an ability to produce betanidin; and
(8) the host has introduced therein a celosianin II synthesis composition.

### Celosianin II Synthesis Composition

A celosianin II synthesis composition (celosianin II synthesis agent or celosianin II synthetase agent) of the present invention contains a gene shown in any one of the following items or a vector carrying the gene:
(1) a gene encoding a polypeptide formed of an amino acid sequence set forth in SEQ ID NO: 57, 59, 61, 63, 65, 67, 69, 2, 4, 6, 8, 10, 12, 14, or 16;
(2) a gene encoding a polypeptide that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16;
(3) a gene encoding a polypeptide that has 90% or more homology to the amino acid sequence set forth in SEQ ID NO: 57, 59, 61, 63, 65, 67, 69, 2, 4, 6, 8, 10, 12, 14, or 16, and that has a substantially equivalent ability to synthesize celosianin II to that of the amino acid sequence set forth in SEQ ID NO: 57, 59, 61, 63, 65, 67, 69, 2, 4, 6, 8, 10, 12, 14, or 16
(4) a gene formed of DNA formed of a base sequence set forth in SEQ ID NO: 56, 58, 60, 62, 64, 66, 68, 1, 3, 5, 7, 9, 11, 13, or 15;
(5) a gene formed of DNA that hybridizes with DNA formed of a base sequence complementary to DNA formed of the base sequence set forth in SEQ ID NO: 56, 58, 60, 62, 64, 66, 68, 1, 3, 5, 7, 9, 11, 13, or 15 under stringent conditions, and that encodes a polypeptide having an ability to synthesize celosianin II;
(6) a gene formed of DNA having a 1- to 50-base sequence substituted, deleted, inserted, and/or added in DNA formed of the base sequence set forth in SEQ ID NO: 56, 58, 60, 62, 64, 66, 68, 1, 3, 5, 7, 9, 11, 13, or 15;
(7) a gene formed of DNA having 90% or more homology to DNA formed of the base sequence set forth in SEQ ID NO: 56, 58, 60, 62, 64, 66, 68, 1, 3, 5, 7, 9, 11, 13, or 15; and
(8) a gene formed of DNA formed of a degenerate isomer of the base sequence set forth in SEQ ID NO: 56, 58, 60, 62, 64, 66, 68, 1, 3, 5, 7, 9, 11, 13, or 15.

Alternatively, the celosianin II synthesis composition of the present invention has a peptide represented by an amino acid sequence of any one of the following items (1) to (3):
(1) an amino acid sequence set forth in SEQ ID NO: 57, 59, 61, 63, 65, 67, 69, 2, 4, 6, 8, 10, 12, 14, or 16;
(2) an amino acid sequence that has 1 to 20 amino acids substituted, deleted, inserted, and/or added in the amino acid sequence set forth in SEQ ID NO: 57, 59, 61, 63, 65, 67, 69, 2, 4, 6, 8, 10, 12, 14, or 16, and that forms a polypeptide having a substantially equivalent ability to synthesize celosianin II to that of the amino acid sequence set forth in SEQ ID NO: 57, 59, 61, 63, 65, 67, 69, 2, 4, 6, 8, 10, 12, 14, or 16; and
(3) an amino acid sequence that has 90% or more homology to the amino acid sequence set forth in SEQ ID NO: 57, 59, 61, 63, 65, 67, 69, 2, 4, 6, 8, 10, 12, 14, or 16, and that forms a polypeptide having a substantially equivalent ability to synthesize celosianin II to that of the amino acid sequence set forth in SEQ ID NO: 57, 59, 61, 63, 65, 67, 69, 2, 4, 6, 8, 10, 12, 14, or 16.

### Amyloid-β Polymerization Inhibitor or Therapeutic or Preventive Agent for Alzheimer's

An amyloid-β polymerization inhibitor or a therapeutic or preventive agent for Alzheimer's of the present invention contains any one or more of the following:
(1) celosianin II;
(2) celosianin II obtained by the method of synthesizing celosianin II described herein;
(3) celosianin II obtained from the celosianin II-producing host described herein;
(4) amaranthin;
(5) betanin;
(6) ferulic acid;
(7) a betaxanthin;
(8) a betaxanthin obtained by the method of synthesizing a betaxanthin described herein; and
(9) a betaxanthin obtained from the betaxanthin-producing host described herein.

### HIV-1 Protease Activity Inhibitor

An HIV-1 protease activity inhibitor of the present invention contains any one or more of the following:
(1) celosianin II;
(2) celosianin II obtained by the method of synthesizing celosianin II described herein; and
(3) celosianin II obtained from the celosianin II-producing host described herein.

### Amyloid Peptide Aggregation Inhibitor

An amyloid peptide aggregation inhibitor of the present invention contains any one or more of the following:
(1) celosianin II;
(2) celosianin II obtained by the method of synthesizing celosianin II described herein;
(3) celosianin II obtained from the celosianin II-producing host described herein;
(4) amaranthin;
(5) betanin;
(6) ferulic acid; and
(7) a betaxanthin.

It has been able to be recognized through Examples to be described later that the amyloid peptide aggregation inhibitor of the present invention inhibits the formation of aggregates of an amyloid peptide, thereby being effective for the prevention and/or treatment of a neurodegenerative disease, Alzheimer's disease, systemic amyloidosis, Parkinson's disease, Huntington's disease, a prion disease, multiple sclerosis, and amyotrophic lateral sclerosis.

### Betaxanthin Synthesis System

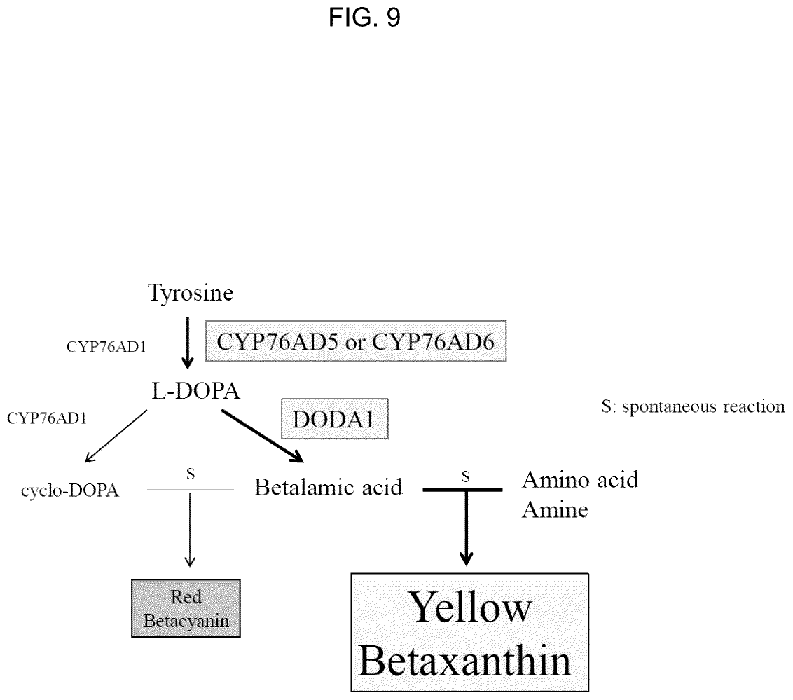

Schemes for betaxanthin biosynthetic pathways in the present invention are illustrated in FIG. 7 and FIG. 9.

As apparent from the illustrations in FIG. 7 and FIG. 9, when a host having an ability to produce tyrosine or 3-hydroxy-L-tyrosine, and an amino acid or an amine (including a host capable of incorporating these compounds from outside) has the following enzyme activities, a betaxanthin can be synthesized.

Enzyme (e.g., CqCYP76AD5 or CqCYP76AD6) activity of having enzyme activity of hydroxylating the 3-position of the phenol ring of tyrosine but being free of L-DOPA oxidase activity and DOPA 4,5-dioxygenase (e.g., CqDODA-1) activity.

According to the results of Examples to be described below, a plant body that is a host having introduced therein a gene group of a gene encoding an enzyme (CqCYP76AD5 or CqCYP76AD6) having enzyme activity of hydroxylating the 3-position of the phenol ring of tyrosine but being free of L-DOPA oxidase activity and a gene encoding an enzyme (CqDODA- 1) having DOPA 4,5-dioxygenase activity has an ability to synthesis a betaxanthin.

Alternatively, a betaxanthin may be synthesized as follows: betalamic acid synthesized by a host having the above-mentioned enzyme activity, having an ability to produce tyrosine or 3-hydroxy-L-tyrosine, and being free of an ability to produce an amine is subjected to a spontaneous reaction with an amino acid or an amine as required.

As apparent from the illustration in FIG. 9, the present invention also encompasses the following methods of synthesizing a betaxanthin.
(1) Tyrosine or 3-hydroxy-L-tyrosine is brought into contact with an enzyme having enzyme activity of hydroxylating the 3-position of the phenol ring of tyrosine but being free of L-DOPA oxidase activity to provide L-DOPA, which is then brought into contact with an enzyme having DOPA 4,5-dioxygenase activity to provide betalamic acid, which is then brought into contact with an amine.
(2) A host that has introduced therein a gene group of a gene encoding an enzyme having enzyme activity of hydroxylating the 3-position of the phenol ring of tyrosine but being free of L-DOPA oxidase activity and a gene encoding an enzyme having DOPA 4,5-dioxygenase activity, and that has an ability to produce tyrosine or 3-hydroxy-L-tyrosine and an ability to produce an amino acid and/or an amine is cultured, and a betaxanthin is extracted from the host after the culture.
(3) A host that has introduced therein a gene group of a gene encoding an enzyme having enzyme activity of hydroxylating the 3-position of the phenol ring of tyrosine but being free of L-DOPA oxidase activity and a gene encoding an enzyme having DOPA 4,5-dioxygenase activity, and that has an ability to produce tyrosine or 3-hydroxy-L-tyrosine is cultured, and betalamic acid is extracted from the host after the culture and brought into contact with an amine.

With regard to the “culture” in the method of synthesizing a betaxanthin described above in each of (2) and (3), for example, the host may be cultured in the presence of an exogenous amine corresponding to the target betaxanthin.

In addition, the method of synthesizing a betaxanthin described above in each of (2) and (3) may further include a step of purifying the target betaxanthin from the extracted pigment, or a step of separating the target betaxanthin and a secondary betaxanthin, which is derived from an endogenous amine and/or amino acid, from the extracted pigment.

### Amine

The amine of the present invention may be a naturally occurring amine or a non-naturally occurring amine. Examples thereof include putrescine, spermidine, spermine, methylamine, ethanolamine, piperidine, histamine, dopamine, phenethylamine, tyramine, 3-methoxytyramine, γ-aminobutyric acid, noradrenaline, serotonin, muscimol, ethylamine, piperazine, morpholine, ethylenediamine, diethylenetriamine, triethylenetetramine, tetraethylenepentamine, pentaethylenehexamine, hexamethylenediamine, 1,3-butadien-1-amine, 1,3,5-hexatrien-1-amine, aniline, 4-biphenylamine, amantadine, p-aminobenzoic acid (PABA), anthranilic acid (Ant.), 6-aminoindole (6AI), and o-dianisidine (oDA).

### Betaxanthin

The betaxanthin of the present invention is not particularly limited, and examples thereof include indicaxanthin, vulgaxanthin I, dopaxanthin, a betaxanthin obtained by a condensation reaction between threonine and betalamic acid, putrescine-betaxanthin, spermidine-betaxanthin, spermine-betaxanthin, methylamine-betaxanthin, ethanolamine-betaxanthin, piperidine-betaxanthin, histamine-betaxanthin, dopamine-betaxanthin, phenethylamine-betaxanthin, tyramine-betaxanthin, 3-methoxytyramine-betaxanthin, γ-aminobutyric acid-betaxanthin, noradrenaline-betaxanthin, serotonin-betaxanthin, muscimol-betaxanthin, ethylamine-betaxanthin, piperazine-betaxanthin, morpholine-betaxanthin, ethylenediamine-betaxanthin, diethylenetriamine-betaxanthin, triethylenetetramine-betaxanthin, tetraethylenepentamine-betaxanthin, pentaethylene hexamine-betaxanthin, hexamethylenediamine-betaxanthin, 1,3-butadien-1-amine-betaxanthin, 1,3,5-hexatrien-1-amine-betaxanthin, aniline-betaxanthin, 4-biphenylamine-betaxanthin, and amantadine-betaxanthin.

### Enzyme Having Enzyme Activity of Hydroxylating 3-Position of Phenol Ring of Tyrosine but Being Free of L-DOPA Oxidase Activity

The enzyme having enzyme activity of hydroxylating the 3-position of the phenol ring of tyrosine but being free of L-DOPA oxidase activity in the present invention may be derived from any species as long as the enzyme has activity of allowing a hydroxy group to be added to the 3-position of the phenol ring that tyrosine has, and is free of L-DOPA oxidase activity.

Examples of the enzyme having enzyme activity of hydroxylating the 3-position of the phenol ring of tyrosine but being free of L-DOPA oxidase activity include cytochromes P450 (in particular, a subfamily β of a CYP76AD group, such as CYP76AD5 and CYP76AD6). Of those, CqCYP76AD5 {CqCYP76AD5a has a base sequence set forth in SEQ ID NO: 50 (accession number: XM_021920495) and an amino acid sequence set forth in SEQ ID NO: 51 (accession number: XP_021776187), and CqCYP76AD5b has a base sequence set forth in SEQ ID NO: 52 (accession number: XM_021861483) and an amino acid sequence set forth in SEQ ID NO: 53 (accession number: XP_021717175)} or CqCYP76AD6 {having a base sequence set forth in SEQ ID NO: 54 (accession number: XM_021861500) and an amino acid sequence set forth in SEQ ID NO: 55 (accession number: XP_021717192)} is preferred.

Examples of the gene encoding the enzyme having enzyme activity of hydroxylating the 3-position of the phenol ring of tyrosine but being free of L-DOPA oxidase activity may include the following:
(1) a gene encoding a polypeptide formed of an amino acid sequence set forth in SEQ ID NO: 51, 53, or 55;
(2) a gene encoding a polypeptide that has 1 to 20 amino acids substituted, deleted, inserted, and/or added in the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(3) a gene encoding a polypeptide that has 90% or more homology to the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(4) a gene formed of DNA formed of a base sequence set forth in SEQ ID NO: 50, 52, or 54;
(5) a gene formed of DNA that hybridizes with DNA formed of a base sequence complementary to DNA formed of the base sequence set forth in SEQ ID NO: 50, 52, or 54 under stringent conditions, and that encodes a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity;
(6) a gene formed of DNA having a 1- to 50-base sequence substituted, deleted, inserted, and/or added in DNA formed of the base sequence set forth in SEQ ID NO: 50, 52, or 54;
(7) a gene formed of DNA having 90% or more homology to DNA formed of the base sequence set forth in SEQ ID NO: 50, 52, or 54; and
(8) a gene formed of DNA formed of a degenerate isomer of the base sequence set forth in SEQ ID NO: 50, 52, or 54.

The gene of the above-mentioned item (2) is a gene encoding a polypeptide having introduced therein such a mutation as not to cause the loss of the ability to hydroxylate a 3-position of a phenol ring of tyrosine. Such mutation encompasses an artificial mutation as well as a naturally occurring mutation. As means for causing the artificial mutation, there may be given, for example, a site-directed mutagenesis method (Nucleic Acids Res. 10, 6487-6500, 1982). The number of mutated amino acids is generally 20 or less, preferably 10 or less, more preferably 5 or less, most preferably 3. Whether or not the polypeptide having introduced therein the mutation retains the ability to synthesize amaranthin can be found by, for example, introducing a gene encoding the polypeptide having introduced therein the mutation into a plant body or the like, and checking the ability to synthesize amaranthin in the plant body.

With regard to the gene of the above-mentioned item (3), the “substantially equivalent ability to hydroxylate a 3-position of a phenol ring of tyrosine to that of the amino acid sequence set forth in SEQ ID NO: 51, 53, or 55” may be stronger or weaker in degree of the action as compared to the substantially equivalent ability to synthesize amaranthin to that of the amino acid sequence set forth in SEQ ID NO: 51, 53, or 55. The degree of the action may be, for example, about 50%, about 60%, about 70%, about 80%, about 90%, about 100%, about 110%, about 120%, about 130%, about 140%, or about 150% as compared to the ability of the amino acid sequence set forth in SEQ ID NO: 51, 53, or 55 to synthesize amaranthin.

In addition, the identity may be calculated using the Basic Local Alignment Search Tool at the National Center for Biological Information (BLAST) or the like (using, for example, default, namely initially set, parameters).

The gene of the above-mentioned item (5) is a gene obtained by utilizing hybridization between DNAs. The term “stringent conditions” in this gene refers to conditions under which only specific hybridization occurs and non-specific hybridization does not occur. Such conditions are generally conditions such as hybridization in a buffer containing 5×SSC and 1% SDS at 37° C. and washing treatment with a buffer containing 1×SSC and 0.1% SDS at 37° C., preferably conditions such as hybridization in a buffer containing 5×SSC and 1% SDS at 42° C. and washing treatment with a buffer containing 0.5×SSC and 0.1% SDS at 42° C., more preferably conditions such as hybridization in a buffer containing 5×SSC and 1% SDS at 65° C. and washing treatment with a buffer containing 0.2×SSC and 0.1% SDS at 65° C. Whether or not DNA obtained by utilizing hybridization encodes a polypeptide having activity can be found by, for example, introducing the DNA into a plant body or the like, and checking the ability of the plant body to synthesize amaranthin. The DNA obtained by hybridization generally has high identity to the gene of the above-mentioned item (4) (SEQ ID NO: 50, 52, or 54). The “high identity” refers to 90% or more identity, preferably 95% or more identity, more preferably 98% or more identity.

The gene of the above-mentioned item (6) is a gene formed of DNA having a 1- to 50-base sequence, preferably a 1- to 30-base sequence, more preferably a 1- to 20-base sequence, most preferably a 1- to 10-base sequence, even most preferably a 1- to 5-base sequence substituted, deleted, inserted, and/or added in DNA formed of the base sequence set forth in SEQ ID NO: 50, 52, or 54.

The gene of the above-mentioned item (7) is a gene formed of DNA having 90% or more, preferably 93% or more, more preferably 95% or more, most preferably 98% or more identity to DNA formed of the base sequence set forth in SEQ ID NO: 50, 52, or 54.

The enzyme having enzyme activity of hydroxylating the 3-position of the phenol ring of tyrosine but being free of L-DOPA oxidase activity in the present invention has any one or more amino acid sequences selected from the following:
(1) an amino acid sequence set forth in SEQ ID NO: 51, 53, or 55;
(2) an amino acid sequence that has 1 to 20 amino acids substituted, deleted, inserted, and/or added in the amino acid sequence set forth in SEQ ID NO: 51, 53, or 55, and that forms a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity; and
(3) an amino acid sequence that has 90% or more homology to the amino acid sequence set forth in SEQ ID NO: 51, 53, or 55, and that forms a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity.

In the introduction of a mutation into a peptide, for example, a substitution between homologous amino acids (e.g., polar amino acids, non-polar amino acids, hydrophobic amino acids, hydrophilic amino acids, positively charged amino acids, negatively charged amino acids, and aromatic amino acids) is easily conceivable from the viewpoint of preventing basic properties (e.g., physical properties, function, physiological activity, or immunological activity) of the peptide from being changed.

### Method of Synthesizing Betaxanthin

A method of synthesizing a betaxanthin of the present invention may be exemplified by the following.
(1) A host that has introduced therein a gene group of a gene encoding an enzyme having enzyme activity of hydroxylating the 3-position of the phenol ring of tyrosine but being free of L-DOPA oxidase activity and a gene encoding an enzyme having DOPA 4,5-dioxygenase activity, and that has an ability to produce tyrosine or 3-hydroxy-L-tyrosine and an ability to produce an amino acid and/or an amine is cultured, and a betaxanthin is extracted from the host after the culture.
(2) A host that has introduced therein a gene group of a gene encoding an enzyme having enzyme activity of hydroxylating the 3-position of the phenol ring of tyrosine but being free of L- DOPA oxidase activity and a gene encoding an enzyme having DOPA 4,5-dioxygenase activity, and that has an ability to produce tyrosine or 3-hydroxy-L-tyrosine is cultured, and betalamic acid is extracted from the host after the culture and brought into contact with an amine.

### Betaxanthin-Producing Host

A betaxanthin-producing host of the present invention has, for example, any one or more of the following configurations and features:
(1) the host has introduced therein a gene encoding an enzyme having enzyme activity of hydroxylating the 3-position of the phenol ring of tyrosine but being free of L-DOPA oxidase activity and a gene encoding an enzyme having DOPA 4,5-dioxygenase activity, and has an ability to produce tyrosine or 3-hydroxy-L-tyrosine and an ability to produce an amino acid and/or an amine; and
(2) the host has an ability to produce an amino acid and/or an amine, and has introduced therein a betalamic acid synthesis composition for betaxanthin synthesis.

### Betalamic Acid-Producing Host

A betalamic acid-producing host of the present invention has, for example, any one or more of the following configurations and features:
(1) the host has introduced therein a gene encoding an enzyme having enzyme activity of hydroxylating the 3-position of the phenol ring of tyrosine but being free of L-DOPA oxidase activity and a gene encoding an enzyme having DOPA 4,5-dioxygenase activity, and has an ability to produce tyrosine or 3-hydroxy-L-tyrosine; and
(2) the host has introduced therein a betalamic acid synthesis composition for betaxanthin synthesis.

### Betalamic Acid Synthesis Composition for Betaxanthin Synthesis

A betalamic acid synthesis composition for betaxanthin synthesis (betalamic acid synthesis agent for betaxanthin synthesis, or betalamic acid synthetase agent for betaxanthin synthesis) of the present invention contains a gene shown in any one of the following items or a vector carrying the gene:
(1) a gene encoding a polypeptide formed of an amino acid sequence set forth in SEQ ID NO: 51, 53, or 55;
(2) a gene encoding a polypeptide that has 1 to 20 amino acids substituted, deleted, inserted, and/or added in the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(3) a gene encoding a polypeptide that has 90% or more homology to the amino acid sequence set forth in SEQ ID NO: 51, 53, or 55, and that has a substantially equivalent ability to hydroxylate a 3-position of a phenol ring of tyrosine to that of the amino acid sequence set forth in SEQ ID NO: 51, 53, or 55 and is free of L-DOPA oxidase activity;
(4) a gene formed of DNA formed of a base sequence set forth in SEQ ID NO: 50, 52, or 54;
(5) a gene formed of DNA that hybridizes with DNA formed of a base sequence complementary to DNA formed of the base sequence set forth in SEQ ID NO: 50, 52, or 54 under stringent conditions, and that encodes a polypeptide having an ability to hydroxylate a 3-position of a phenol ring of tyrosine and being free of L-DOPA oxidase activity;
(6) a gene formed of DNA having a 1- to 50-base sequence substituted, deleted, inserted, and/or added in DNA formed of the base sequence set forth in SEQ ID NO: 50, 52, or 54;
(7) a gene formed of DNA having 90% or more homology to DNA formed of the base sequence set forth in SEQ ID NO: 50, 52, or 54; and
(8) a gene formed of DNA formed of a degenerate isomer of the base sequence set forth in SEQ ID NO: 50, 52, or 54.

Alternatively, the betalamic acid synthesis composition for betaxanthin synthesis of the present invention has a peptide represented by an amino acid sequence of any one of the following items (1) to (3):
(1) an amino acid sequence set forth in SEQ ID NO: 51, 53, or 55;
(2) an amino acid sequence that has 1 to 20 amino acids substituted, deleted, inserted, and/or added in the amino acid sequence set forth in SEQ ID NO: 51, 53, or 55, and that forms a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity; and
(3) an amino acid sequence that has 90% or more homology to the amino acid sequence set forth in SEQ ID NO: 51, 53, or 55, and that forms a polypeptide having a substantially equivalent ability to hydroxylate a 3-position of a phenol ring of tyrosine to that of the amino acid sequence set forth in SEQ ID NO: 51, 53, or 55 and being free of L-DOPA oxidase activity.

### EXAMPLES

The present invention is hereinafter described in detail by way of specific examples. However, the present invention is not limited to these Examples.

### Example 1

### Stability of Betalain Pigment

An aqueous solution of each of celosianin II, amaranthin, and betanin was evaluated for its stability at 37° C.

### Betalain Pigment Extraction and HPLC Analysis

For amaranthin and betanin extraction, amaranthin-synthesizing tobacco BYII cells and betanin-synthesizing tobacco BYII cells generated in accordance with a previously reported method were used (Non Patent Literature 4). For celosianin II extraction, a wild-type quinoa individual was used. Each plant sample having celosianin II, amaranthin, or betanin accumulated in the plant body was disrupted, and water was added, followed by centrifugation (20,000 g, 10 minutes or 15 minutes). The centrifuged supernatant was collected, and an equal amount of acetonitrile was added, followed by centrifugation (20,000 g, 10 minutes or 15 minutes). The supernatant obtained by the centrifugal operation was concentrated with a centrifugal concentrator (CC-105, Tomy Seiko). The resultant concentrate was subjected to HPLC analysis as a pigment extract.

The HPLC analysis was performed by: using a reversed-phase column (Shim-pack GWS C18 column, 5 μm; 200 mm×4.6 mm i.d.; Shimadzu GLC); using 0.05% TFA in water as solvent A and 0.05% TFA in acetonitrile as solvent B; setting a flow rate to 0.5 ml/min; setting an analysis program to such a condition that the concentration of acetonitrile was linearly changed from 0% at an analysis time of 0 minutes to reach 45% over 45 minutes; and setting an analytical wavelength to 536 nm allowing betacyanin to be detected.

The purified betalain pigments were subjected to measurement with a UV-2450 spectrophotometer (Shimadzu), followed by the calculation of the concentrations of solutions using the molar extinction coefficient of amaranthin and betanin (ε=54,000 M⁻¹ cm⁻¹ at 536 nm) (Gandia-Herrero, F., Escribano, J. and Garcia-Carmona, F. (2010) Structural implications on color, fluorescence, and antiradical activity in betalains. Planta 232, 449-460.; Schwartz, S. J. and Von Elbe, J. H. (1980) Quantitative determination of individual betacyanin pigments by high-performance liquid chromatography. J. Agric. Food Chem. 28, 540-543.).

### With Regard to Stability of Betalain Pigment

For each of betanin, amaranthin, and celosianin II, a 1 mM aqueous solution was prepared, and left to stand still in an incubator at 37° C. Absorbances at 536 nm on days 0, 1, 2, 3, and 4 were measured, and for the resultant values, relative amounts with respect to the absorbance on day 0 were calculated.

### Results

According to the results of the measurement of the residual amounts of the betalain pigments with the absorbances at 536 nm serving as indicators, celosianin was more abundant than amaranthin and betanin. Further, in the case of 4 days of treatment at 37° C., the red colors of betanin and amaranthin disappeared, but celosianin II still exhibited a red color.

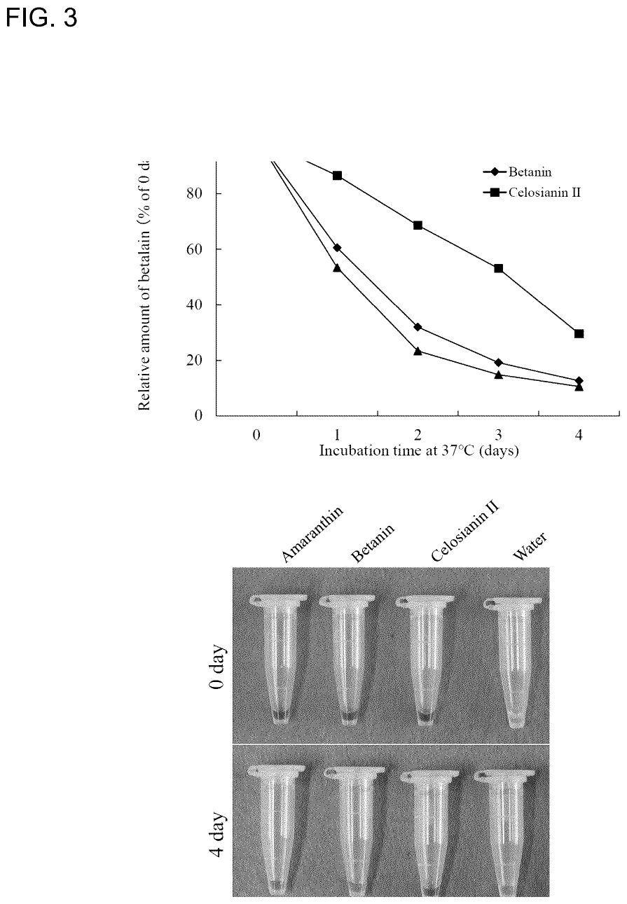

Thus, it was revealed that celosianin II was stable as compared under the condition of 37° C. as compared to amaranthin and betanin (FIG. 3).

### Example 2

### Effect of Celosianin II on HIV-1 Protease Activity

Whether celosianin II inhibited HIV-1 protease activity was tested.

### Evaluation of HIV-1 Protease Inhibitory Activity of Betalain Pigment

A recombinant HIV-1 protease (ab84117) from Abcam was used as an HIV-1 protease, and an HIV-1 protease substrate (Lys-Ala-Arg-Val-Nle-p-nitro-Phe-Glu-Ala-Nle amide) from Sigma-Aldrich was used as a substrate peptide for the HIV-1 protease. HIV-1 protease activity evaluation was performed with reference to a previously reported method (Boso, G., Orvell, C. and Somia, N.V. (2015) The nature of the N-terminal amino acid residue of HIV-1 RNase H is critical for the stability of reverse transcriptase in viral particles. J. Virol. 89, 1286-1297.). Amaranthin, betanin, or celosianin II was added to 16 pmol of the HIV-1 protease and 4 nmol of the HIV-1 protease substrate, a buffer (25 mM NaCl, 25 mM Na₂HPO₄, 1 mM dithiothreitol, pH 4.7) was added so that the amount of a reaction liquid was 30 μl, and a reaction was performed at 25° C. for 2 hours. With regard to the addition amount of amaranthin, betanin, or celosianin II, amaranthin, betanin, and celosianin II were each added at the ratio of a 100-fold amount with respect to the amount of the HIV-1 protease. For celosianin II, the ratios of 25-and 50-fold amounts were further evaluated. An HIV-1 inhibitor saquinavir was used as a positive control.

After the reaction, the reaction liquid was measured for the amount of the residual substrate peptide through use of HPLC, and a residual ratio with respect to the substrate before the reaction was calculated. For the HPLC, LC-20AD from Shimadzu was utilized, and a Shim-pack GWS C18 column (5 μm; 200 mm×4.6 mm i.d.; Shimadzu GLC) was used as an analytical column. For an analytical solvent system, 0.05% TFA in water was used as solvent A and 0.05% TFA in acetonitrile was used as solvent B. An HPLC program was set to a linear gradient from 0% of solvent B at 0 minutes to 50% of solvent B at 50 minutes, and the HPLC was performed at a flow rate of 0.5 mL/min and 25° C. Analysis was performed with an analytical wavelength being set to 260 nm allowing the HIV-1 protease substrate peptide to be detected.

### Results

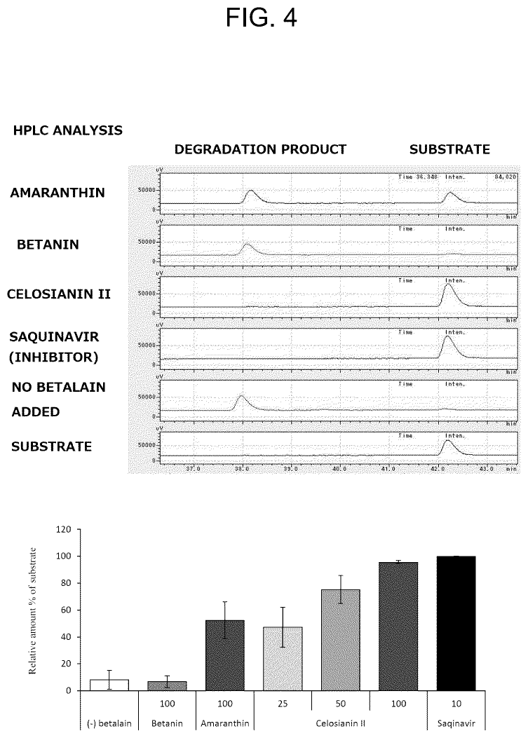

It was revealed that, when added in a 25-fold amount with respect to the HIV-1 protease, celosianin II had HIV-1 protease inhibitory activity comparable to that in the case where a 100-fold amount of amaranthin was added (FIG. 4). That is, it was recognized that celosianin II had an HIV-1 protease activity inhibitory effect 4 times as high as that of amaranthin. Further, the inhibitory activity of celosianin II on the HIV-1 protease was amount-dependent (FIG. 4).

### Example 3

### Effect of Betalain Pigment on Amyloid-β Polymerization

With regard to the polymerization of amyloid-β, there is no report about a betalain pigment. The inventors of the present invention evaluated whether celosianin II, amaranthin, and betanin pigments, and ferulic acid, a constituent component of celosianin II, inhibited the polymerization of amyloid-β by a ThT assay method.

### Evaluation of Amyloid-β Polymerization Inhibitory Activity in Thioflavin T (ThT) Assay

ThT assay involved evaluating polymerization using SensoLyte Thioflavin T β-Amyloid (1-40) Aggregation Kit (ANASPEC) in accordance with an accompanying protocol.

As an apparatus used for measurement, Varioskan LUX (Thermo Fisher Scientific) was used, and fluorescence derived from the polymerization of amyloid-β was measured for 4 hours at intervals of 5 minutes. A sample was stirred for 15 seconds before each measurement and subjected to the measurement. An excitation wavelength of 440 nm and a detection wavelength of 484 nm were used.

The sample used for the ThT assay was prepared to have a final concentration of 50 μM before being used for the experiment. Tannic acid (Ono, K., Hasegawa, K., Naiki, H. & Yamada, M. (2004) Anti-amyloidogenic activity of tannic acid and its activity to destabilize Alzheimer's beta-amyloid fibrils in vitro. Biochim Biophys Acta 1690, 193-202.) was used as a control of an inhibitor.

### Results

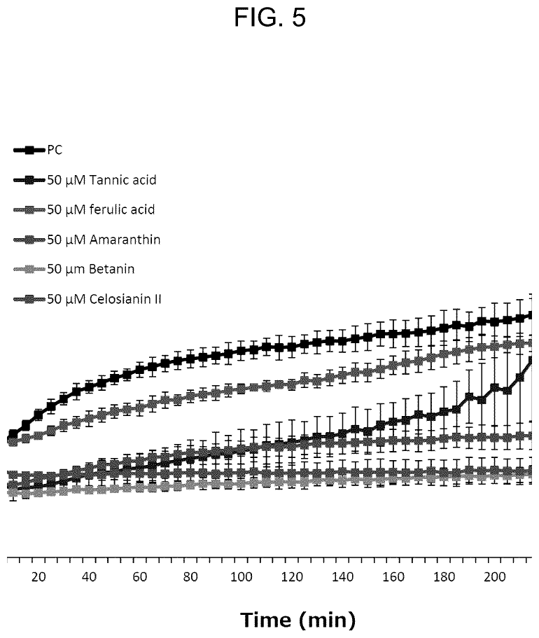

Celosianin II and betanin each showed polymerization inhibitory activity comparable to or higher than that of the existing inhibitor (tannic acid) (FIG. 5). In addition, amaranthin possessed inhibitory activity, though its effect was low as compared to celosianin II and betanin (FIG. 5). Meanwhile, in the case of only ferulic acid, a constituent component of celosianin II, the polymerization inhibitory effect was weaker than those of celosianin II, the betanin pigment, and amaranthin (FIG. 5).

Thus, it was recognized that the betalain pigments each had activity of inhibiting amyloid polymerization.

### Example 4

### Search for Celosianin II Synthetase Gene

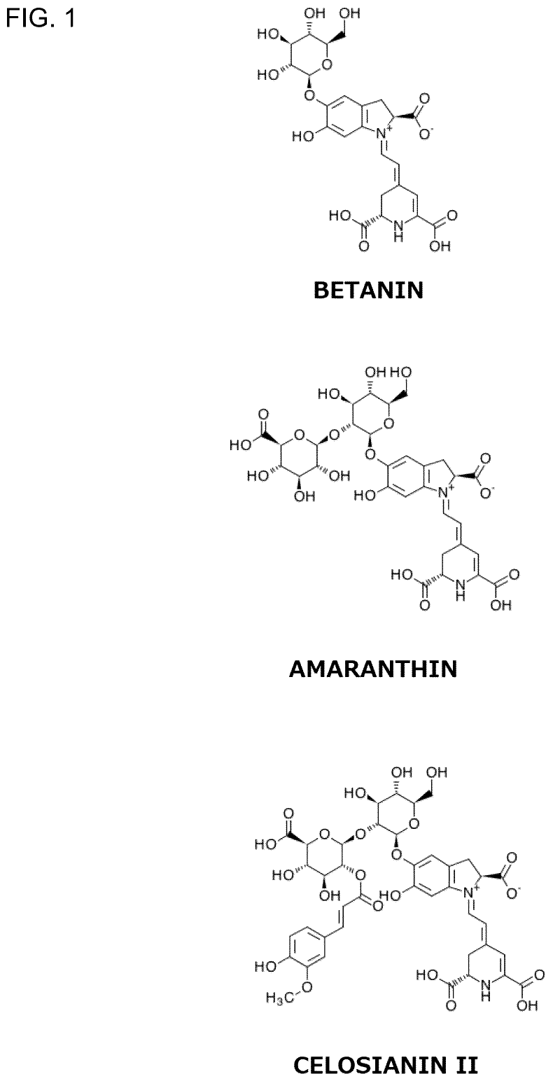

In order to isolate a celosianin II-synthesizing gene from a quinoa genome, a celosianin II synthetase gene was identified on the basis of an intracellular localization prediction and gene expression analysis. Celosianin II is a substance in which ferulic acid is bonded to glucuronic acid present in the molecule of amaranthin (FIG. 1). In a flavonoid, another plant pigment, enzymes that add ferulic acid analogs, such as sinapinic acid, p-coumaric acid, and gallic acid, to the flavonoid have already been isolated (Bontpart, T., Cheynier, V., Ageorges, A. & Terrier, N. (2015) BAHD or SCPL acyltransferase? What a dilemma for acylation in the world of plant phenolic compounds. New Phytol 208, 695-707.). It is known that the enzymes (acyltransferases) that add those molecules are proteins belonging to the Serine CarboxyPeptidase Like (SCPL) group, and are localized in vacuoles (Bontpart, T., Cheynier, V., Ageorges, A. & Terrier, N. (2015) BAHD or SCPL acyltransferase? What a dilemma for acylation in the world of plant phenolic compounds. New Phytol 208, 695-707.). In view of this, in order to isolate a celosianin II-synthesizing gene from quinoa, first, 87 SCPL group proteins present in the quinoa genome were analyzed using an intracellular localization prediction program (WoLF PSORT).

### Vacuolar Localization Prediction

SCPL group proteins registered at NCBI were analyzed using the intracellular localization prediction program (WoLF PSORT, https://wolfpsort.hgc.jp/). On the basis of the results of the analysis, proteins each having a vacuolar localization score of 4 or more were selected as vacuole-localized proteins.

### Comprehensive Expression Analysis in Quinoa Hypocotyls

RNA was extracted from hypocotyls of quinoa (Chenopodium quinoa) on day 5 of germination through use of an RNeasy Plant Mini kit (Qiagen). A library for next-generation sequencing was constructed from 1 μg of the extracted RNA through use of NEBNext Poly(A) mRNA Magnetic Isolation Module (NEB). The constructed library was sequenced using MiSeq (Illumina). The resultant next-generation sequencing data was aligned to quinoa genomic data through use of HISAT2 (Kim, D., Langmead, B. & Salzberg, S. L. (2015) HISAT: a fast spliced aligner with low memory requirements. Nat Methods 12, 357-360.). A gene prediction was performed using String Tie (Pertea, M. et al. (2015) String Tie enables improved reconstruction of a transcriptome from RNA-seq reads. Nat Biotechnol 33, 290-295.), and an expression amount was determined using feature Counts (Liao, Y., Smyth, G. K. & Shi, W. (2014) feature Counts: an efficient general purpose program for assigning sequence reads to genomic features. Bioinformatics 30, 923-930.).

### Results

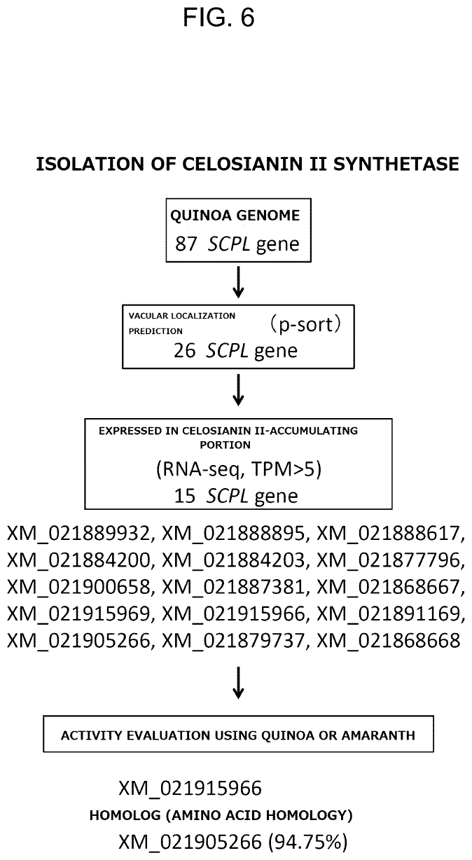

According to the results of the vacuolar localization prediction, there were 26 proteins predicted to be localized in vacuoles (FIG. 6).

Celosianin II is accumulated in hypocotyls, and hence it is predicted that a celosianin II-synthesizing gene is also expressed in hypocotyls. In view of this, as the next selection, the 26 genes predicted to be localized in vacuoles were examined for expression in hypocotyls. For the selection, RNA-seq was performed for hypocotyls of quinoa. On the basis of the resultant RNA-seq data, genes expressed in the hypocotyls were selected (Transcripts Per Kilobase Million (TPM)>5). As a result, it was revealed that 15 genes were expressed in the hypocotyls (FIG. 6).

### Example 5

### Evaluation of Celosianin II Synthesis Activity of Celosianin II-synthesizing Gene

For the 15 selected candidate genes, the intensities of activity of synthesizing celosianin II were compared using an evaluation system using quinoa and amaranth.

### Generation of Plasmid for Plant Expression

For the expression of betalain pigment biosynthesis genes used in this experiment, a pCAMBIA1301 modification vector (Imamura, T., Takagi, H., Miyazato, A., Ohki, S., Mizukoshi, H. and Mori, M. (2018) Isolation and characterization of the betalain biosynthesis gene involved in hypocotyl pigmentation of the allotetraploid Chenopodiumquinoa. Biochem. Biophys. Res. Commun. 496, 280-286.) was used. For the betalain pigment biosynthesis genes, a fragment in which restriction enzyme sites were added to the 5′ and 3′ ends of each gene was synthesized by PCR. Then, the resultant PCR fragment was cleaved at the added sites and inserted into the plant modification vector to construct a plant expression vector.

### Agrobacterium Transformation Method

The constructed plasmid was introduced from Escherichia coli harboring the plasmid into an Agrobacterium using a triparental mating method (Wise, A. A., Liu, Z. and Binns, A. N. (2006) Three methods for the introduction of foreign DNA into Agrobacterium. Methods Mol. Biol. 343, 43-53.) to generate a transformed Agrobacterium.

### Evaluation of Celosianin Synthesis Activity Using Transient Plant Expression System

Whether a selected candidate gene possessed an ability to synthesize celosianin II was evaluated. For the experiment, quinoa and amaranth (which does not synthesize celosianin II), which is an Amaranthaceae plant like quinoa and performs synthesis up to amaranthin, a precursor of celosianin II, were used. Those plant bodies were each infected with the transformed Agrobacterium to transiently express the candidate gene in the plant body, and the synthesis of celosianin II in the infected plant body was evaluated.

A plasmid for expressing the selected candidate gene in a plant is constructed and introduced into an Agrobacterium. The resultant transformed Agrobacterium was subjected to shaking culture at 120 rpm in 3 mL of LB medium at 26° C. for 1 day. 3 ml of the culture liquid in which the Agrobacterium was sufficiently proliferated was diluted with sterilized water to 20 mL, and 20 μL of 10 mg/mL acetosyringone and 10 μL of Tween 20 (surfactant) were added to prepare an infected bacterial liquid. A plant tissue of quinoa or amaranth was placed in the infected bacterial liquid, and a pressure reduction operation was performed using a desiccator for from 10 minutes to 15 minutes to allow the bacterium to infiltrate the plant tissue. The infiltrated plant tissue was transferred to an MS plate and subjected to static culture in a dark place at 23° C. for from 3 days to 4 days. A betalain pigment was extracted from the cultured plant tissue, and the ability of the candidate gene to synthesize celosianin II was evaluated by HPLC analysis.

### Results

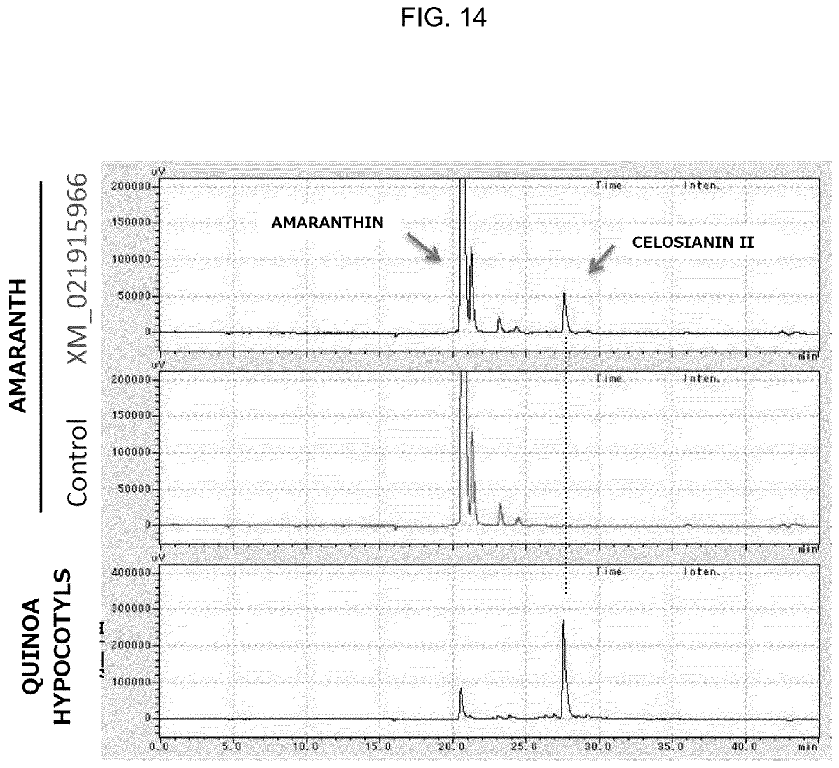

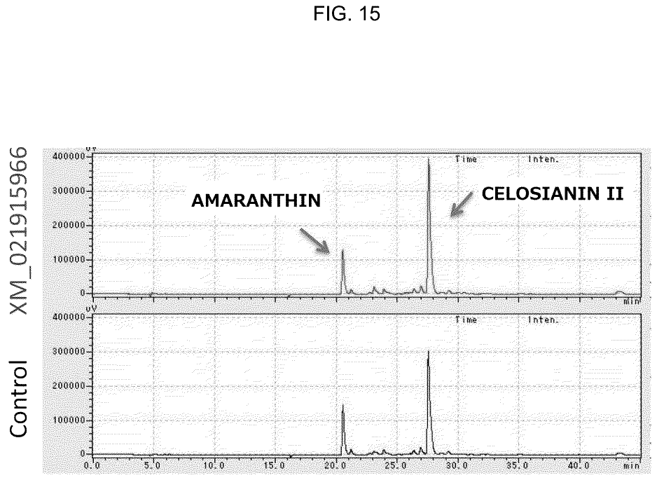

According to the results of the pigment analysis, the accumulation of celosianin II, which was not originally observed in amaranth, was able to be found in the amaranth caused to transiently express a gene (XM_021915966: SEQ ID NOS: 56 and 57) (FIG. 14). Further, in the quinoa caused to have a transient expression, the accumulation amount of celosianin II was increased about 2-fold as compared to the control (FIG. 15). Besides, XM_021905266 (SEQ ID NOS: 58 and 59) has high homology to XM_021915966 (amino acid homology: 94.75%), and hence has a similar effect. Further, 13 kinds belonging to the same SCPL gene family as XM_021915966 (XM_021889932, XM_021888895, XM_021888617, XM_021884200, XM_021884203, XM_021877796, XM_021900658, XM_021887381, XM_021868667, XM_021915969, XM_021891169, XM_021879737, and XM_021868668) also have similar effects.

### Example 6

### Mass Production of Celosianin II in Tobacco Cultured Cells (BY-2 Cells)

The celosianin II synthetase gene was successfully isolated, and hence an attempt is made to mass produce celosianin II in tobacco BY-2 cells. In order to produce amaranthin, vectors for expression in BY-2 cells are constructed, and transformants having introduced therein five kinds of betalain biosynthesis genes (CqCYP76AD1-1, CqDODA-1, CqCDOPA5GT, CqAmaSy1, and celosianin II synthetase) are generated. A strongly red line is selected from the resultant transformed BY-2 lines.

### Analysis of Constitutive Expression in Tobacco BY2 Cells

The plasmid harbored by the transformed Agrobacterium is introduced into tobacco BY2 cells through use of an Agrobacterium method (Hagiwara, Y., Komoda, K., Yamanaka, T., Tamai, A., Meshi, T., Funada, R., Tsuchiya, T., Naito, S. and Ishikawa, M. (2003) Subcellular localization of host and viral proteins associated with tobamovirus RNA replication. EMBO J. 22, 344-353.). The resultant transformed line is maintained and utilized for other analyses.

### Results

The selected celosianin II-producing line is subjected to HPLC and mass spectrometry to determine the production of celosianin II. In addition, a celosianin II production amount in 150 mL culture is investigated. Through this experiment, betalain pigments (amaranthin and betanin) can be mass produced in tobacco BY-2 cultured cells, i.e., a non-betalain-producing plant.

### Example 7

### Betaxanthin Production in Tobacco Cultured Cells (BY-2 Cells)

Through a homology search (Blastp) using the amino acid sequences of beet (Beta vulgaris)-like genes {BvCYP76AD5 (NCBI accession number: AJD87473) and BvCYP76AD6 (AJD87474)}, CqCYP76AD5 (XM_021920495, XM_021861483), and CqCYP76AD6 (XM_021861500) were selected and identified. Of those, XM 021920495 was used for expression in BY-2 cells.

In order to produce a betaxanthin, a vector for expression in BY-2 cells was constructed, and transformants having introduced therein two kinds of betaxanthin biosynthesis genes (CqCYP76AD5 and CqDODA-1) were generated. From the resultant transformed BY-2 lines, lines exhibiting yellow to orange colors were selected.

### Analysis of Constitutive Expression in Tobacco BY2 Cells

The plasmid harbored by the transformed Agrobacterium was introduced into tobacco BY2 cells through use of an Agrobacterium method (Hagiwara, Y., Komoda, K., Yamanaka, T., Tamai, A., Meshi, T., Funada, R., Tsuchiya, T., Naito, S. and Ishikawa, M. (2003) Subcellular localization of host and viral proteins associated with tobamovirus RNA replication. EMBO J. 22, 344-353.). The resultant transformed line was maintained and utilized for analyses.

### Results

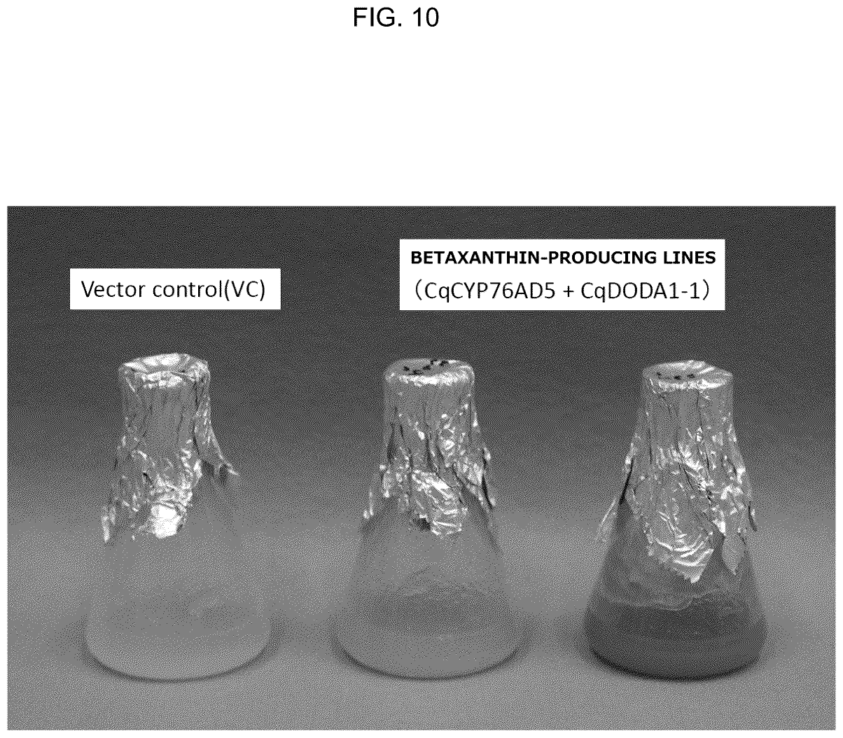

The results are shown in FIG. 10.

The tobacco BY-2 cells transformed with CqCYP76AD5 and CqDODA1-1 were changed in color to yellow or orange, indicating that a betaxanthin was synthesized. The expression amounts of CqCYP76AD5 and CqDODA1-1 differ depending on differences in numbers and insertion sites of genes of interest inserted into the genome of the BY-2 cells in transformation, and hence the lines had colors, such as yellow and orange, different from each other. An orange line has a larger amount of a betaxanthin accumulated therein than a yellow one. The tobacco BY2 cells having an empty vector introduced therein remained white, indicating that no betaxanthin was synthesized.

The synthesized betaxanthin is a mixture of betaxanthins, and hence the betaxanthins are identified by performing HPLC and mass spectrometry.

### Example 8

### Activity Evaluation of Betaxanthin Synthesized in Cells

The activity of a betaxanthin extracted from the betaxanthin-synthesizing tobacco BY-2 cells generated in Example 7 was evaluated.

### Betaxanthin Extraction

A BY-2 cell sample having the betaxanthin accumulated in the plant body was disrupted, and ethanol was added, followed by centrifugation (20,000 g, 10 minutes or 15 minutes). The centrifuged supernatant was collected, and a 4-fold amount of ethanol was added, followed by centrifugation (20,000 g, 10 minutes or 15 minutes). The supernatant obtained by the centrifugal operation was concentrated with a centrifugal concentrator (CC-105, Tomy Seiko). The resultant concentrate was used as a betaxanthin extract.

### Effect of Betaxanthin on Amyloid- β Polymerization

With regard to the polymerization of amyloid-β, no report about a betaxanthin has been found. Accordingly, whether the betaxanthin inhibits the polymerization of amyloid-β was evaluated by a ThT assay method.

### Evaluation of Amyloid-β Polymerization Inhibitory Activity in Thioflavin T (ThT) Assay

ThT assay involved evaluating polymerization using SensoLyte Thioflavin T β-Amyloid (1-40) Aggregation Kit (ANASPEC) in accordance with an accompanying protocol.

As an apparatus used for measurement, Varioskan LUX (Thermo Fisher Scientific) was used, and fluorescence derived from the polymerization of amyloid-β was measured for 4 hours at intervals of 5 minutes. A sample was stirred for 15 seconds before each measurement and subjected to the measurement. An excitation wavelength of 440 nm and a detection wavelength of 484 nm were used.

The sample used for the ThT assay was prepared to have a final concentration of 50 μM before being used for the experiment. An extracted sample from the BY-2 cells having an empty vector introduced therein (VC) and water (PC) in place of a sample were used as controls. In addition, betanin and curcumin were used as positive controls. Curcumin is a plant-derived substance reported to have amyloid-β polymerization inhibitory activity (Yang F, Lim GP, Begum AN, et al. Curcumin inhibits formation of amyloid betaoligomers and fibrils, binds plaques, and reduces amyloid in vivo. J BiolChem. 2005; 280(7): 5892-5901. doi:10.1074/jbc.M404751200).

### Results

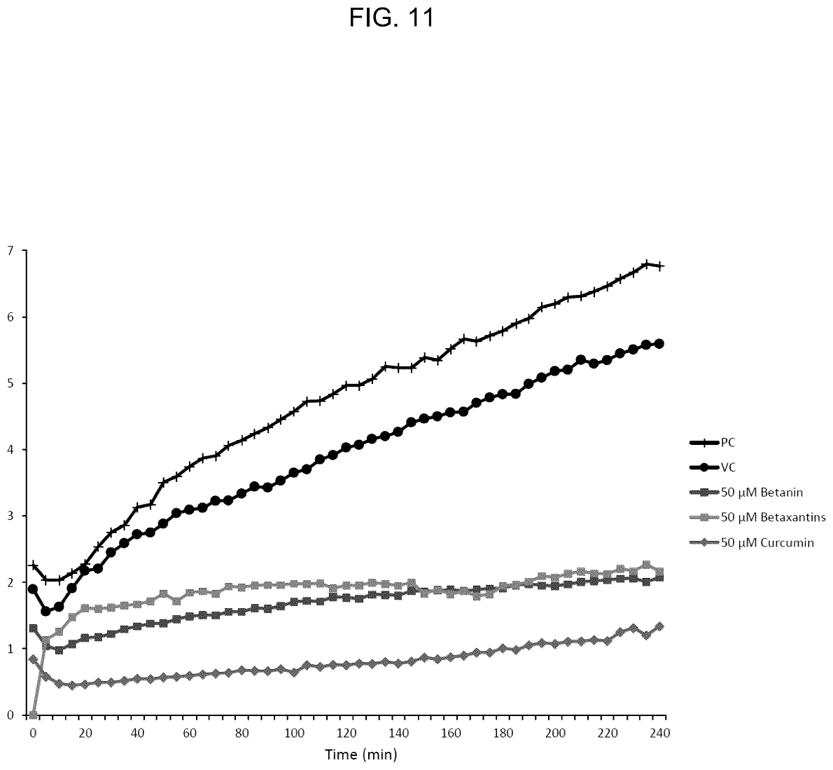

According to the results of the ThT assay, the betaxanthin possessed inhibitory activity comparable to that of betanin (FIG. 11).

Thus, it was recognized that the betaxanthin had activity of inhibiting amyloid polymerization.

### Example 9

### Observation of Amyloid Peptide Using Transmission Electron Microscope

The influences of betalain pigments on the aggregation of an amyloid peptide were observed with a transmission electron microscope.

A lyophilized amyloid peptide (human amyloid-β 1-40, Aβ40) was purchased from Peptide Institute, Inc., dissolved as a stock solution at a concentration of 1 mM using dimethyl sulfoxide, and stored at-20° C. until use.

As a betaxanthin, the betaxanthin extracted from the betaxanthin-producing BY2 cells (FIG. 10) was used.

With regard to reaction conditions, Aβ40 and a sample (betanin, amaranthin, celosianin II, ferulic acid, or the betaxanthin) were prepared with PBS (pH 7.4) so as to have a final concentration of 25 μM and a final concentration of 50 μM, respectively. As a control, distilled water was used in place of the sample. The prepared sample was left to stand still in a dark place at 37° C. for 5 days.

The sample after the reaction was stained on a grid mesh for a transmission electron microscope by adding about 2 μL of a 2% uranyl acetate solution thereto. After the solution had dried, the aggregation of the amyloid peptide was observed using a transmission electron microscope (Hitachi 7650 TEM).

### Results

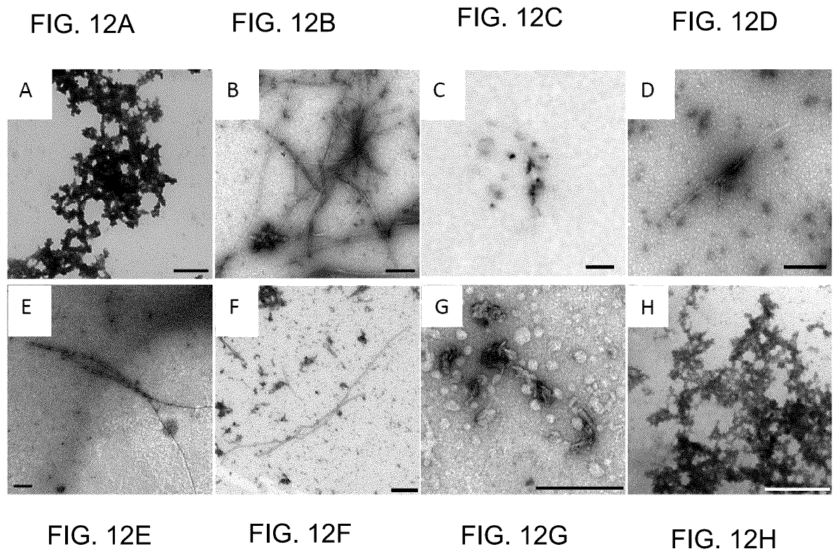

According to the results of the observation of the aggregation of the amyloid peptide with the transmission electron microscope, the control sample having added thereto water in place of a betalain pigment was observed to show amorphous aggregation of the amyloid peptide (FIG. 12A) and amyloid fibrils (FIG. 12B).

Meanwhile, when betanin, amaranthin, celosianin II, or ferulic acid (constituent component of celosianin II) was added, only short amyloid fibrils were observed, and hence the aggregation of the amyloid peptide was inhibited (FIG. 12C to FIG. 12G).

In addition, when the betaxanthin was added, amorphous aggregation was observed, but amyloid fibrils were not observed (FIG. 12H).

From the above-mentioned results, it was able to be recognized that betanin, amaranthin, celosianin, and ferulic acid each had an effect of inhibiting both of the polymerization and amorphous aggregation of amyloid fibrils, and the betaxanthin had an effect of inhibiting the polymerization of amyloid fibrils.

### Example 10

### Test Using Nematodes

In a transgenic nematode expressing an amyloid peptide, aggregates of the amyloid peptide are formed in the body along with its growth period, and the nematode is paralyzed. In view of this, whether feeding with betanin changed a paralysis ratio was evaluated.

Nematodes were grown using NGM plates (nematode growth medium), and grown using Escherichia coli (OP50 strain) as food under the condition of 15° C. unless otherwise indicated.

Transgenic nematodes (CL2006) expressing an amyloid peptide (human amyloid-β 1-42, Aβ42) were obtained from the Caenorhabditis Genetics Center (CGC), USA. The CL2006 expresses Aβ42 in the cells of the body wall muscle of the nematode, and then aggregation of the amyloid peptide occurs in the muscle. As a result, paralytic symptoms are induced in the CL2006 strain (C. D. Link, Expression of human beta-amyloid peptide in transgenic Caenorhabditis elegans, Proc. Natl. Acad. Sci. U.S.A., 92 (1995) 9368-9372.).

Groups of nematode individuals to be used for the experiment were grown on NGM plates for 2-3 generations without starvation in order that all the individuals were in the same growth stage. Young adult nematodes were collected therefrom and cleaned, and the nematodes were disintegrated using a lysis solution (0.6% sodium hypochlorite and 200 mM sodium hydroxide) to isolate eggs. After from 12 hours to 14 hours at 20° C., the isolated eggs were hatched, and the synchronized L1 larvae were used for the subsequent experiment.

The synchronized L1-stage larvae were grown with the food to young adults at 20° C. 5-Fluorodeoxyuridine (0.5 mg/ml) was added to prevent progeny production during the growth.

The individuals grown to adults through the synchronized growth were transferred to NGM plates containing various concentrations (2 μM, 10 μM, and 50 μM) of food having mixed therein dextrin or betanin, and were grown. After the nematodes had been transferred, the nematodes were each evaluated for paralysis at constant intervals. 20 synchronized adult individuals were transferred per plate. Dextrin was used as a control.

Paralysis was evaluated on the basis of the behavior of the nematode after the nose of the nematode had been lightly tapped with a platinum wire. According to a previous report, an individual that, after the nose of the nematode had been tapped, moved its nose but failed to move its body was considered paralyzed (E. Cohen, J. Bieschke, R. M. Perciavalle et al., Opposing activities protect against age-onset proteotoxicity, Science, 313 (2006) 1604-1610.).

### Results

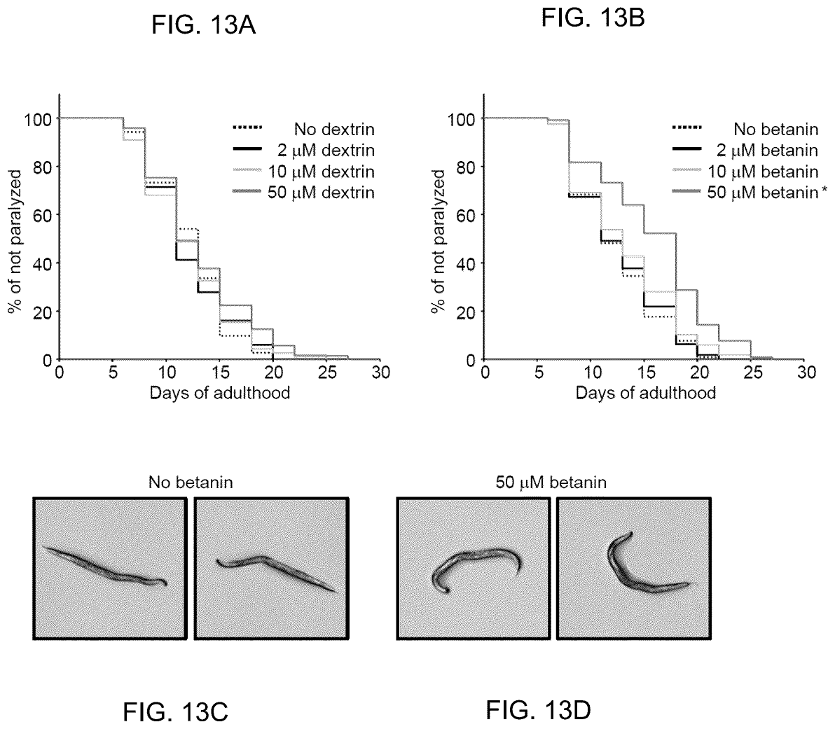

As a result of the experiment using the transgenic nematodes overexpressing the amyloid peptide, the following results were able to be obtained: as compared to the group fed with dextrin (control), the group fed with 50 μM betanin showed significant amelioration (reduction) in paralysis ratio (FIG. 13B). FIG. 13C shows typical examples of paralyzed nematodes in the group fed with dextrin. FIG. 13D shows typical examples of normal nematodes in the group fed with betanin.

According to the above-mentioned results, the following result was able to be obtained: betanin inhibited the aggregation of the amyloid peptide even in vivo.

### Industrial Applicability

According to the present invention, the method of synthesizing celosianin II and the method of synthesizing a betaxanthin can be provided.
1-20. (canceled)

21. A method of inhibiting amyloid peptide aggregation in a subject in need thereof, comprising administering an effective amount of a composition comprising an active ingredient selected from the group consisting of:
(a) Celosianin II;
(b) Betanin;
(c) Betaxanthin; and
(d) Amaranthin.

22. The method of claim 21, wherein the active ingredient is Celosianin II.

23. The method of claim 21, wherein the active ingredient is Betanin.

24. The method of claim 21, wherein the active ingredient is Betaxanthin.

25. The method of claim 21, wherein the active ingredient is Amaranthin.

26. The method of claim 21, wherein the subject has a neurodegenerative disease.

27. The method of claim 26, wherein the neurodegenerative disease is Alzheimer's disease.

28. The method of claim 26, wherein the neurodegenerative disease is Parkinson's disease.

29. The method of claim 26, wherein the neurodegenerative disease is Huntington's disease.

30. The method of claim 26, wherein the neurodegenerative disease is Amyotrophic Lateral Sclerosis.

31. The method of claim 21, wherein the amyloid peptide aggregation is inhibited by inhibiting amyloid fibril formation or amorphous aggregates.

32. The method of claim 21, wherein the amyloid peptide is an amyloid-β peptide.

33. The method of claim 23, wherein administering the composition comprising betanin reduces paralysis associated with amyloid peptide aggregation in the subject.


SEQUENCE LISTING (69 sequences)

SEQ ID NO: 1 - DNA, 1383 bp, Chenopodium quinoa
  source 1..1383: mol_type = genomic DNA
atgttgtctttcacaaaagtcataacctttgcaacccttttattatgcaactttgcattacaagtaactccattatcatctgaagatgataaaatcacaagcttaccagggcaaccccctgttaatttccagcaattttcgggttatattactgtcgacgaagtccaaaaaagaaatcttttttactatttagttcaagctgaaactaacccttcttccaaaccccttgttctttggctcaatggagggccaggttgttcatctattggagcaggagcattcagtgaacatggaccctttagaccaagtggaaatggattgattaagaatgaatttagctggaataaagaagcaaacatgttgtatcttgaatcaccagcaggagttgggttttcttattctgctaataaaacattttatgatggagttaatgatgaaatgacagcaagagataatctggtatttcttcaaaaattcctagcaaaatacccagaatacaagaatagagatttattcctcactggagagagctatggaggacattatgttccacaacttgctaacctcgtgctaaaatcaagcctcaagtcaaatttgaggggagttgctataggaaatccattgctagagttcaacaatgacttcaattcaatcggtgaatatctttggtctcatggactaatatctgatgcaacttatgatctttctgaaaaaatctgcaattattcacaaattaggagacaagaacagaaattcctttcagcaccttgcaaattagttaattctcaaattaatagtgagattggcaagtttattgactcgtacgatgtcactctcgacgtgtgtttacattcggtttctcaacaagctcatgtcctcaaccaaatgatggatgaagagaaaattgatgtttgtctagatgatgaaacaacccaatacttgaataggaaagatgtacaaaaggctatgcatgcacaccttattggggtaactcaatgggctacttgtagcaatgtgctaaactatcacatggacgatttggagattcctactcttccactacttggcactttcactaaaagcggcgtgagagttctggtttacagtggtgaccaagattcaataataccgttcattggaactagaacgcttgtaaacggattggcaaaggagttgggactaaacactactgtgccttatcgaacttggctaacaaatagacaggttggtggttggacgcaagtatatggtgacattcttgcatatgcaactataagaggagcttcccatgaagcaccattttcgcaacctgctcgatctctcgtgctcttccgctccttcctggatgggaagccacttccagatgtctcacttgctcaataa

SEQ ID NO: 2 - protein, 460 aa, Chenopodium quinoa
MLSFTKVITFATLLLCNFALQVTPLSSEDDKITSLPGQPPVNFQQFSGYITVDEVQKRNLFYYLVQAETNPSSKPLVLWLNGGPGCSSIGAGAFSEHGPFRPSGNGLIKNEFSWNKEANMLYLESPAGVGFSYSANKTFYDGVNDEMTARDNLVFLQKFLAKYPEYKNRDLFLTGESYGGHYVPQLANLVLKSSLKSNLRGVAIGNPLLEFNNDFNSIGEYLWSHGLISDATYDLSEKICNYSQIRRQEQKFLSAPCKLVNSQINSEIGKFIDSYDVTLDVCLHSVSQQAHVLNQMMDEEKIDVCLDDETTQYLNRKDVQKAMHAHLIGVTQWATCSNVLNYHMDDLEIPTLPLLGTFTKSGVRVLVYSGDQDSIIPFIGTRTLVNGLAKELGLNTTVPYRTWLTNRQVGGWTQVYGDILAYATIRGASHEAPFSQPARSLVLFRSFLDGKPLPDVSLAQ

SEQ ID NO: 3 - DNA, 1440 bp, Chenopodium quinoa
  source 1..1440: mol_type = genomic DNA
atgtctatattacttgttttttttttcattagtttctttcttaagtgtagcaaaatgtcgtctttcacaaaactcataacttttgtaacccttttattatgcaactttgcattacaagtaactccattatcatctcaagatgataaaatcacaagcttaccagggcaaccccctgttaatttccagcaattttctggctatattactgtcaacgaagtcgaaaaaagaaatcttttttactatttagttcaagctgaaactaacccttcttccaaaccccttgttctttggctcaatggagggccaggttgctcatctattggagcaggagcatttagtgaacatggaccctttagaccaagtggaaatggattgattaagaatgagtttagttggaataaagaagcaaatatgttgtatcttgaatcaccagcaggagttgggttttcttattctgctaataaatcattttataatggagttaatgatgaaatgacagcaagagataatctggtatttcttcaaaaattcctagcaaaatacccagaatacaagaatagagatttgttcctcactggagagagctatggaggacattatgttccacaacttgctaacctcattctaaaatcaagcctcaagtcaaatttgaagggagttgctataggaaatccactggtggagtttaacactgatttcaattcaaggacagaatatatttggtctcatggacttatttcagatgcaacatatgagctttctgaaaagatatgcaattattcacaaattaggagacaagcacagaaaggatttctctcagcaccttgcaaattagttatttcacaaattaatagtgagattggcaagtttattgactcgtacgatgtcactctcgacgtttgtttacattcggtttctcaacaagctcatgtactacgccaaatgatggatgaagagaaaattgatgtttgtctagatgatgaaacaacccaatacttgaatagaaaagatgtacaaaaggctatgcatgcaaaccttattggggtaactcaatgggctacttgtagcaatgtgctcaactatgacatggacgatttggaggttcctactcttccactacttggtacattcactaaaagtggcgtaagagttttggtttacagtggtgatcaagattcagtaataccattcattggaactagaacgcttgtaaacggattggcaaaggagttgggactaaacactacggttccttatagaacttggctagcgaatagacaggttggtggatggacgcaagtgtatggtgacattcttgcatatgcaactgtaagaggagctgcccacgaagtaccattttcgcaacccgctcgatctctcgtgctcttccgctccttcctggacgggaagccactaccagatgtctcacttgctcaataa

SEQ ID NO: 4 - protein, 479 aa, Chenopodium quinoa
MSILLVFFFISFFLKCSKMSSFTKLITFVTLLLCNFALQVTPLSSQDDKITSLPGQPPVNFQQFSGYITVNEVEKRNLFYYLVQAETNPSSKPLVLWLNGGPGCSSIGAGAFSEHGPFRPSGNGLIKNEFSWNKEANMLYLESPAGVGFSYSANKSFYNGVNDEMTARDNLVFLQKFLAKYPEYKNRDLFLTGESYGGHYVPQLANLILKSSLKSNLKGVAIGNPLVEFNTDFNSRTEYIWSHGLISDATYELSEKICNYSQIRRQAQKGFLSAPCKLVISQINSEIGKFIDSYDVTLDVCLHSVSQQAHVLRQMMDEEKIDVCLDDETTQYLNRKDVQKAMHANLIGVTQWATCSNVLNYDMDDLEVPTLPLLGTFTKSGVRVLVYSGDQDSVIPFIGTRTLVNGLAKELGLNTTVPYRTWLANRQVGGWTQVYGDILAYATVRGAAHEVPFSQPARSLVLFRSFLDGKPLPDVSLAQ

SEQ ID NO: 5 - DNA, 1362 bp, Chenopodium quinoa
  source 1..1362: mol_type = genomic DNA
atgaaaaataccaaatttggttgttttttcttggtttttctgggtattttttctgctacatttgcaaatcaagagaaagatagaataacatggttaccaggccaaccagaaaatgtgaattttgctcaatattctggttatgtaacagtagataaatcagctggtaaagcattgttttattggttaactgaatcaccttcaaatccagagtcaaaaccattgttattatggcttaatggcggtcctggttgttcatcagttgcttatggtgctgcagaagagattggtccatttcatattaaaaatgatggaaaaagtctttacttgaataaatattcatggaataaggaggcaaatttgctatttcttgagtctcctgttggtgttgggttctcatactcaaatagatcatctgatttgtatgctcttggtgatagcaggacagctaaagatgcctataaatttcttgttaaatggttcaataggtttccccagtacaagcacagagaattctacattgctggggaaagttatgcagggcattatgttcctcaattgtctcaacttatttacagaagaaacaaaggaattgcacaaccagaaataaacttcaagggatttatggtgggaaatgcagttacagatgattattatgattacattggtacatttgagtactggtggacacatggtttaatttcagattccacctacaagagccttaaaacagcttgtttggctggatcatctcagcatcctagtattgtatgcaacagagctctggaaatggctgattctgagttcggaaacatcgatccatacagtatctatacccgtccttgcaactcgactgcctcgatgaagcgcagtctaaggggccgttatccttggatgacgagagcatacgatccatgcaccgagaggtactcgaaccagtacttcaatcttcctgaagttcaactcgctatgcatgctaatgtgaccaaattgtcttatccatggagcacttgcagtgacatagttggaacttactggtctgactctccacaatcgatgcttccaatttatcgagaactcatagcaggaggcttgaaaatatgggtattcagcggcgacactgatgctgtagtgcctattacagcaactcgatattctattgatgcactgagactccctactgtgaacaattggtccccttggtatgacaatggaagggttggtggatggagccaagaatacaagggtctaacatatgtgacaataacaggagcaggacatgaagtacctcttcatagacctcgacaagcttttatacttttcaatcatttcttaaaggacagatctatgccaagccgaagctaa

SEQ ID NO: 6 - protein, 453 aa, Chenopodium quinoa
MKNTKFGCFFLVFLGIFSATFANQEKDRITWLPGQPENVNFAQYSGYVTVDKSAGKALFYWLTESPSNPESKPLLLWLNGGPGCSSVAYGAAEEIGPFHIKNDGKSLYLNKYSWNKEANLLFLESPVGVGFSYSNRSSDLYALGDSRTAKDAYKFLVKWFNRFPQYKHREFYIAGESYAGHYVPQLSQLIYRRNKGIAQPEINFKGFMVGNAVTDDYYDYIGTFEYWWTHGLISDSTYKSLKTACLAGSSQHPSIVCNRALEMADSEFGNIDPYSIYTRPCNSTASMKRSLRGRYPWMTRAYDPCTERYSNQYFNLPEVQLAMHANVTKLSYPWSTCSDIVGTYWSDSPQSMLPIYRELIAGGLKIWVFSGDTDAVVPITATRYSIDALRLPTVNNWSPWYDNGRVGGWSQEYKGLTYVTITGAGHEVPLHRPRQAFILFNHFLKDRSMPSRS

SEQ ID NO: 7 - DNA, 1428 bp, Chenopodium quinoa
  variation 196: note = n is a, c, g, or t
  source 1..1428: mol_type = genomic DNA
atgatgagcagcataactaaaattctttctcttctcatctttattgtattctatattacattcattaaagcatctgaaacattcgctgtgtcagaagaagatgacagcaagatactaaatttaccaggtcaaccatcaaacccaccaatatcacagtactcaggttacatcactgtcaaccaaaataatggcaggnccctattttactggttctttgaagctctttcacagccttccgataaacctctcatactctggctgaatggaggacctggttgctcatcagtaggatatggcgctgttgtggaattaggtcctcttaaagttgatgaaactggtgttggtttgctcttcaataacttttcatggataacagaagcaaatttgctcttcgtggagtcccctgttggagtagggttctcttataccaacacatcctctgacctcaccaaattggatgatgaatttgtagctgaggatgcatacagtttcttagtaaattggtttgggagatacccacagttcaagaaccaagacttcttcctagccggagaaagttatgcaggtcactatgttcctcaactggcagagcttatttttgacaggaacaaggatcagactatgaacctgaagattaatctcaaaggtttcctagtagggaaccctgaaactgatgatttttatgactcaaagggtctgttagaatatgcttggagtcacactgtaataagtgatgatatgtataataagactaaagaagtgtgtgatttcaagaccttatattggccgaaggaatgcgtgactgccatgaatttggtttatggaaaatacaaagaaatcgacttttacaacatatatgctccaatttgtctttcaaacagcacttcatcagaagcttattatactaataaggaggaaaattatggattgaggaggccaagaatatattcaggggggtatgacccttgctattcaaactacgcagaaacgtacttcaacagggcagatgtacaaacatcgcttcgtgcaaatgctaacagagggacttcaaatactagtgtcaagtggaatgtttgcaacgacaacattctgaatacatacaacgctacagttcaatctgtcctatcaatctacaagaaactgatgcaaggtggtcttaagatctgggtttacagcggagatgtcgatggaagagtcccggtgattggaactaggtactgggttgagtcacttgggttgccaatcaagacaccttggcgttcctggtaccatcaacatcaggtaggaggaagattagtggagtacgaaggtttaactttggtgactgttcgaggggcaggtcatttggtacctctgaataagcctagtgaagcacttgctctgattcactcttttttaacaggagaagatcttccagcaaatagatga

SEQ ID NO: 8 - protein, 475 aa, Chenopodium quinoa
  VARIANT 66: note = Xaa can be any naturally occurring amino acid
MMSSITKILSLLIFIVFYITFIKASETFAVSEEDDSKILNLPGQPSNPPISQYSGYITVNQNNGRXLFYWFFEALSQPSDKPLILWLNGGPGCSSVGYGAVVELGPLKVDETGVGLLFNNFSWITEANLLFVESPVGVGFSYTNTSSDLTKLDDEFVAEDAYSFLVNWFGRYPQFKNQDFFLAGESYAGHYVPQLAELIFDRNKDQTMNLKINLKGFLVGNPETDDFYDSKGLLEYAWSHTVISDDMYNKTKEVCDFKTLYWPKECVTAMNLVYGKYKEIDFYNIYAPICLSNSTSSEAYYTNKEENYGLRRPRIYSGGYDPCYSNYAETYFNRADVQTSLRANANRGTSNTSVKWNVCNDNILNTYNATVQSVLSIYKKLMQGGLKIWVYSGDVDGRVPVIGTRYWVESLGLPIKTPWRSWYHQHQVGGRLVEYEGLTLVTVRGAGHLVPLNKPSEALALIHSFLTGEDLPANR

SEQ ID NO: 9 - DNA, 1428 bp, Chenopodium quinoa
  source 1..1428: mol_type = genomic DNA
atgatgagcagcataactaaaattctttctcttctcatctttattgtattctatattacattcattaaagcatctgaaacattcgctgtgtcagaagaagatgacagcaagatactaaatttaccaggtcaaccatcaaacccaccaatatcacagtactcaggttacatcactgtcaaccaaaataatggcagggccctattttactggttctttgaagctctttcacagccttccgataaacctctcatactctggctgaatggaggacctggttgctcatcagtaggatatggcgctgttgtggaattaggtcctcttaaagttgatgaaactggtgttggtttgctcttcaataacttttcatggataacagaagcaaatttgctcttcgtggagtcccctgttggagtagggttctcttataccaacacatcctctgacctcaccaaattggatgatgaatttgtagctgaggatgcatacagtttcttagtaaattggtttgggagatacccacagttcaagaaccaagacttcttcctagccggagaaagttatgcaggtcactatgttcctcaactggcagagcttatttttgacaggaacaaggatcagactatgaacctgaagattaatctcaaaggtttcctagtagggaaccctgaaactgatgatttttatgactcaaagggtctgttagaatatgcttggagtcacactgtaataagtgatgatatgtataataagactaaagaagtgtgtgatttcaagaccttatattggccgaaggaatgcgtgactgccatgaatttggtttatggaaaatacaaagaaatcgacttttacaacatatatgctccaatttgtctttcaaacagcacttcatcagaagcttattatactaataaggaggaaaattatggattgaggaggccaagaatatattcaggggggtatgacccttgctattcaaactacgcagaaacgtacttcaacagggcagatgtacaaacatcgcttcgtgcaaatgctaacagagggacttcaaatactagtgtcaagtggaatgtttgcaacgacaacattctgaatacatacaacgctacagttcaatctgtcctatcaatctacaagaaactgatgcaaggtggtcttaagatctgggtttacagcggagatgtcgatggaagagtcccggtgattggaactaggtactgggttgagtcacttgggttgccaatcaagacaccttggcgttcctggtaccatcaacatcaggtaggaggaagattagtggagtacgaaggtttaactttggtgactgttcgaggggcaggtcatttggtacctctgaataagcctagtgaagcacttgctctgattcactcttttttaacaggagaagatcttccagcaaatagatga

SEQ ID NO: 10 - protein, 475 aa, Chenopodium quinoa
MMSSITKILSLLIFIVFYITFIKASETFAVSEEDDSKILNLPGQPSNPPISQYSGYITVNQNNGRALFYWFFEALSQPSDKPLILWLNGGPGCSSVGYGAVVELGPLKVDETGVGLLFNNFSWITEANLLFVESPVGVGFSYTNTSSDLTKLDDEFVAEDAYSFLVNWFGRYPQFKNQDFFLAGESYAGHYVPQLAELIFDRNKDQTMNLKINLKGFLVGNPETDDFYDSKGLLEYAWSHTVISDDMYNKTKEVCDFKTLYWPKECVTAMNLVYGKYKEIDFYNIYAPICLSNSTSSEAYYTNKEENYGLRRPRIYSGGYDPCYSNYAETYFNRADVQTSLRANANRGTSNTSVKWNVCNDNILNTYNATVQSVLSIYKKLMQGGLKIWVYSGDVDGRVPVIGTRYWVESLGLPIKTPWRSWYHQHQVGGRLVEYEGLTLVTVRGAGHLVPLNKPSEALALIHSFLTGEDLPANR

SEQ ID NO: 11 - DNA, 1512 bp, Chenopodium quinoa
  source 1..1512: mol_type = genomic DNA
atggctaaaccttgtcattatattcttaatcacatattaataccaattttaatggtgttttttctctgttttataattactgaatctgcccctcaaagttctattgttactcaacttcctggatttaatggcacttttccctccaaacactactctgggtatgtgacaattgatgagagtcacgggaagaaattgttttactactttgttgaatcagagagaaacccatcagaggatcctgttgttctttggcttaatggaggacccggatgctcgagtttagatggcttcgtgtacgagcatggtcctttcaattttgaggcagcaaagacaaaaggaggcttgccaaagttgcatattaatccatacagctggtctaaggtttccaatgttatatacttggattctccagcaggagttgggtattcttatgcacaaaacattagtgagtatgataccggggaccttaagaccgcctctgatacccatacattcctgctcaagtggtttgatatctataaagagtttgttgcaaacccatttttcatcgcgggtgagtcttatgctgggatttatgtgccaacccttgcttcagatgttgtgaaaggacttgatgccggtgtaaagcctgttatcaatttaaagggttatttggtaggaaatggtgttacagatgaaacctttgatggaaatgctttagtgccttttgcacatggaatgggcttgatctctgatcaattatatcaggaggttgtcactcagtgtgaaggaaactattacaatctgaaatctgatagttgtgatgccttgctttcaaaagttaaccaggatgttgatggattgaatatatatgatattctggagccttgttatcactcccctgactcagatatgcagagtactgctcatctaagactaccttcgaaaatttccagcttcaaggcattaggagaaactgaaagacctcttcctgtgaggaaaagaatttttggccgtgcttggcccctcagagctcctgtgagagatggacatgttcctacctggccggaacttgctaacagcgcttctgttccttgcattaatgatgaagttgcaactttgtggctgaatgacaaggcagtgagaaaagcaatccatgctgaagaggaaagtgtgtcgggtccctgggaactttgcacaggcaggatttcatttagccatgatgctggaagcatgattacataccataagaacctcacttctcgaggttatcgagccctcatatacagtggagaccatgatatgtgtgttccatacactgggagcgaggcatggacaaaatctgttgggtacaaagtagtagatgattggaggccatgggtttgcaatgaacaagtttctgggttcacacaggggtatgagaagaatctcactttccttactataaagggggcaggacatacggtaccagaatacaaaccgcgagaagcactgtacttttatagccagtttctggcagggaaaagtatataa

SEQ ID NO: 12 - protein, 503 aa, Chenopodium quinoa
MAKPCHYILNHILIPILMVFFLCFIITESAPQSSIVTQLPGFNGTFPSKHYSGYVTIDESHGKKLFYYFVESERNPSEDPVVLWLNGGPGCSSLDGFVYEHGPFNFEAAKTKGGLPKLHINPYSWSKVSNVIYLDSPAGVGYSYAQNISEYDTGDLKTASDTHTFLLKWFDIYKEFVANPFFIAGESYAGIYVPTLASDVVKGLDAGVKPVINLKGYLVGNGVTDETFDGNALVPFAHGMGLISDQLYQEVVTQCEGNYYNLKSDSCDALLSKVNQDVDGLNIYDILEPCYHSPDSDMQSTAHLRLPSKISSFKALGETERPLPVRKRIFGRAWPLRAPVRDGHVPTWPELANSASVPCINDEVATLWLNDKAVRKAIHAEEESVSGPWELCTGRISFSHDAGSMITYHKNLTSRGYRALIYSGDHDMCVPYTGSEAWTKSVGYKVVDDWRPWVCNEQVSGFTQGYEKNLTFLTIKGAGHTVPEYKPREALYFYSQFLAGKSI

SEQ ID NO: 13 - DNA, 1437 bp, Chenopodium quinoa
  source 1..1437: mol_type = genomic DNA
atgagtaagctagcatcaagggttatagcttactatgttgaagtggtagtaatattgatgagtttggtatcattggcattatccaattatcctcatcatgccacaacaaaaacatcggaattatattctacaattgttgattttctgcctggatttcccggaaaactccctttcactcttgaaactgggtacattgaagtttgggaagaagtgcagctattttactacttcatacaatctgagcaaaacccagaaatagatccaattttgatttggtttgctggaggtcctggttgttcttcctttactgatcttgcttttgggcatattgggcctttatccttcaactttacgggaattaatttcaactatgcaacatcaagttgggaggttgatctaccaagacttcaattaaatccttattcttggaccaaggttgcaaacgtaatatttatagattcacctgctggcgctggattctcctatgcaactaacaaatcttattaccctagtgatacaaagacatcaaaacaagcacatgggtttttgaagaagtggcttattgagcatcctaaatttgcaagaaaccctctttacattgctggtgcttcatatgggggaaaagttgttccatctcttacatgggagattgtaaaaggaaatgaagctggaattgggccacaaatgaatcttcagggatatattcttgtaaatccaataactgcttggtaccttgatgaccaaaagaaagttgaatatgctcatcgcatctctcttatatccgatgaactatacaagtctgccaaattaagctgcaatgaagattatcggacaaatgaggctaataagagtaatgcacaatgcaacaagattcttgaagatgtatcaaattgcattgatccactagaagaccaacatgtactggatcctaaatgcacccattcttcttcgccaaatcattggtgtcggggaaatgatcgtatgattgctagccagtgggcaaataacattaatgtccaagaagctcttcatgttcgagagggaactattagccagtggattcgttgtcgcggtcacgcaaaatctgatgtgtttatgtacaataaagatatcgacagtagcatcatttatcatcagaatcttaccacccaacctctcagggcactaatcttcagtggtgaccaagatatgagtgttccatatgtgactacactaaaatggatcaaggagcttaatatcgccccaaatgatgattattggaagccttggtatcttaataatggccaagtttcaggatatgtaactgaatattccaatggctacttcaatttgacatttactacaatcaagggagcagggcatgttgcctccgagtataagccgcgggaatgttttacaatgtttcaaagatggatcacatttagtcctctatcataa

SEQ ID NO: 14 - protein, 478 aa, Chenopodium quinoa
MSKLASRVIAYYVEVVVILMSLVSLALSNYPHHATTKTSELYSTIVDFLPGFPGKLPFTLETGYIEVWEEVQLFYYFIQSEQNPEIDPILIWFAGGPGCSSFTDLAFGHIGPLSFNFTGINFNYATSSWEVDLPRLQLNPYSWTKVANVIFIDSPAGAGFSYATNKSYYPSDTKTSKQAHGFLKKWLIEHPKFARNPLYIAGASYGGKVVPSLTWEIVKGNEAGIGPQMNLQGYILVNPITAWYLDDQKKVEYAHRISLISDELYKSAKLSCNEDYRTNEANKSNAQCNKILEDVSNCIDPLEDQHVLDPKCTHSSSPNHWCRGNDRMIASQWANNINVQEALHVREGTISQWIRCRGHAKSDVFMYNKDIDSSIIYHQNLTTQPLRALIFSGDQDMSVPYVTTLKWIKELNIAPNDDYWKPWYLNNGQVSGYVTEYSNGYFNLTFTTIKGAGHVASEYKPRECFTMFQRWITFSPLS

SEQ ID NO: 15 - DNA, 1419 bp, Chenopodium quinoa
  source 1..1419: mol_type = genomic DNA
atgattagcagggcttgcatccattggtggctgatgctggtaatttttgttgggattggttgtttgaatgggtatcctctcgaggatattgtggtaggattgcctggccaaccaaaagttggattcaaacagtttgctggctacattgatttggatgttaagactgggaagagcttgttttactactttgttgaagctgagaaagatccagaccataagcctctcactctttggctcaatggaggccctggttgctcgtcacttggtggtggtgcctttacggaattgggaccattctttcctacaggtgatggtaggggtcttcgaaaaaatccaaaatcttggaataaagcatcaaatctcctatttgtggagtctcctgctggagtaggctggtcatattcaaatacaacggctgattacaatgccggggatacctcaacagcaaaagacatgcaactttttatgatgaagtggtatgagaaatttccagctctgaaatcgagggatttctttcttactggagaaagctatgcagggcattacataccacaattagctgttgctttactggatcataatgcccattctactcgttttaagttcaacataaaaggagttgctattggaaatccattgctcagacttgatcgagatactcgggcaacatatgagttcctatggtcacatgggatgatttctgatgaaataggcctcaccataataaaggaatgtgatttcagtgattatagttttggtagtcctgatcataatgtttctggtaaatgctatgatgcaataacagaagctcagctcagcgttagtagctacgttgataactataatgtcattgtagatgtttgttacccatcgatagtggagcaagaatttcggttgagaaaagcggccactaaaataagcttgggtattgatatttgcattaattacgaatctggattctatttcaatcttccagaggtccaaagcgctctccatgcaaatcgaacaaaattgccctacagctggtccatgtgtagcgaagttttgaactataccaagactgacgtcaagatgaatatgcttcccttgcttaaaggaatcatccaaaaaggtgttccggtttgggtttacagcggagatcaagattctgtagttccactgctgggatcccgccagatgattcatgaacttgctcgtgaactaaactttacagtcactgctccttatggagcttggtttcacaaagggcaggtaggaggttggtcaactgagtatggaaatttgttgacctttgccacagtaaggggtgctgcacatatggtgccctattcacagccagccagggctttacacctctttagttcttttgttcgtggtctaagattgcctaattcaacacgcctgcccatagatgactag

SEQ ID NO: 16 - protein, 472 aa, Chenopodium quinoa
MISRACIHWWLMLVIFVGIGCLNGYPLEDIVVGLPGQPKVGFKQFAGYIDLDVKTGKSLFYYFVEAEKDPDHKPLTLWLNGGPGCSSLGGGAFTELGPFFPTGDGRGLRKNPKSWNKASNLLFVESPAGVGWSYSNTTADYNAGDTSTAKDMQLFMMKWYEKFPALKSRDFFLTGESYAGHYIPQLAVALLDHNAHSTRFKFNIKGVAIGNPLLRLDRDTRATYEFLWSHGMISDEIGLTIIKECDFSDYSFGSPDHNVSGKCYDAITEAQLSVSSYVDNYNVIVDVCYPSIVEQEFRLRKAATKISLGIDICINYESGFYFNLPEVQSALHANRTKLPYSWSMCSEVLNYTKTDVKMNMLPLLKGIIQKGVPVWVYSGDQDSVVPLLGSRQMIHELARELNFTVTAPYGAWFHKGQVGGWSTEYGNLLTFATVRGAAHMVPYSQPARALHLFSSFVRGLRLPNSTRLPIDD

SEQ ID NO: 17 - DNA, 1494 bp, Chenopodium quinoa
  source 1..1494: mol_type = genomic DNA
atggatcatgcaacactagcaatgatacttgctatttggtttgttgtttttcatttcattaaaatgttattcactagccaaactaccaaacttcttccccccggtcctaaaccattgccactaatcggaaacatccttgaagttggtgagaaacctcaccagtcatttgctaatcttgctaagattcatggtccattgatatcattacgtctaggaagtgtcacaactattgttgtatcctcggctgaagtagccaaagaaatgttcttaaaaaaagaccacccactttctaaccgcactgttcctaattctgtcactgctggggatcaccataagctgactatgtcgtggttgcctgtttctccaaagtggcggaactttagaaaaatcaccgccgttcatttactttctcctcaaagacttgatgcttgccaaaccctaagacatgccaaagtgcaacagcttttccaatatgtacaagaatgtgcacaaaaaggacaagctgttgatattggcaaggcagcatttacaacctcccttaatttgttatcaaaattattcttttcggtcgaattagcccaccataaatcccatacatctcaacaattcaaagaacttatatggaatattatggaagatattggcaaacctaactacgctgattattttccaatcttaggatgcttagacccatcaggaattcgacgtcgattggcgtctaattttgacaagttgattgcagtttttcaaagcataatatgtcaaaggattggcaacggccaggattctgcttcgacgaagacgaccgatgatgtgctagacattcttcttgacctccacaaacaaaaagagctcagtatgggcgagataaatcatctgctcgtggatatttttgatgctgggactgacactacatcaagtacttttgaatgggtaatggcagagttaattcgaaatcctaagatgatggaaaaagctcaagaagaaattgagcaagtcttgggcaaggatagacaaattcaagaatcagacattattaagctaccttacttacaagccattatcaaagaaacattgcggctacacccaccaactgtatttctcttgcctcgtaaagctgatagtgatgttgaattatatggctatgttgtgccgaaagatgcacaaatacttgttaatttatgggccattggaagagaccctcaagcgtgggtgaaacctgacgtgtttttacctgagaggtttttaggatccgaaattgatgtgaaaggaagagattttggactcttaccttttggagctggaaggagaatatgccccgggatgaatttggctattagaatgttaactttgatgttagctacgcttcttcaattcttcaattggaagcttgaagaaggtatgaaagcagaagatctagacatggatgagaaatttgggattgccttacaaaagactaaacctcttcagattattcccgttcttaggtattga

SEQ ID NO: 18 - protein, 497 aa, Chenopodium quinoa
MDHATLAMILAIWFVVFHFIKMLFTSQTTKLLPPGPKPLPLIGNILEVGEKPHQSFANLAKIHGPLISLRLGSVTTIVVSSAEVAKEMFLKKDHPLSNRTVPNSVTAGDHHKLTMSWLPVSPKWRNFRKITAVHLLSPQRLDACQTLRHAKVQQLFQYVQECAQKGQAVDIGKAAFTTSLNLLSKLFFSVELAHHKSHTSQQFKELIWNIMEDIGKPNYADYFPILGCLDPSGIRRRLASNFDKLIAVFQSIICQRIGNGQDSASTKTTDDVLDILLDLHKQKELSMGEINHLLVDIFDAGTDTTSSTFEWVMAELIRNPKMMEKAQEEIEQVLGKDRQIQESDIIKLPYLQAIIKETLRLHPPTVFLLPRKADSDVELYGYVVPKDAQILVNLWAIGRDPQAWVKPDVFLPERFLGSEIDVKGRDFGLLPFGAGRRICPGMNLAIRMLTLMLATLLQFFNWKLEEGMKAEDLDMDEKFGIALQKTKPLQIIPVLRY

SEQ ID NO: 19 - DNA, 1476 bp, Chenopodium quinoa
  source 1..1476: mol_type = genomic DNA
atggagtcatcaacaaaagaacaagaacaacaacacataatcctcctcccatttttagcacaaggccatctccgaccattcctccacctcgctcaccgccttctctctctaacacccttcaatctctctctcctcactacccctttaaacgccgctaatctccgccgtcaatccgacaacttcaacatcaaccttaacatcatcgaactccccttcactagcaccgaccacggtctcccacccaacaccgaaaataccgataaactctcattaacctccatcatcaaactcttccatgcatccacttcactcgaacctcatgtacgtgactacctcacgcgccaccacctgcacgacccacccgtgtgcataatcttcgacgtgttcttgggctgggccgataacgtggcccgatcagttgggtccaccggaatctgcttcaacactggtggggcctacggtgtcggtgcgtatgtttcaatctggagcaatctacctcaccggaatgtcggcgatgatgaggagttttcattggctggtttcccggaagatcggaaactccgacgtaatcagcttcatcggtttttaaggtttgctgatgggtccgatgaatggtcgaggtttttccaacctcagattaaatcatccttgaattgttctgggtggttgtgtaattccatcgaggagattgaaccgttagggtttcaagtttttaggaatttcacgaaatctcctatttggggaattggtcctttgattatgacatcttcgaaaaaagatgatgatgagaaggaagaggcttgtttgaggtggttgaatcaatttgaaaatgactcagttttgtacatttgtttcgggtcacagaatacagtgactccgattcagatgatggagttagcgaaaggtttggaggagagtaagatcccgttcttgtgggtgattcggccgccattcgggttcgatttcaatggggagtttaaacccgaatggctgcctgagaagtttgaggaaagaatgatggagaaaaaacaggggatgttggtgagagattgggtgcctcagttggatattttgaggcatgaggctactggtggatttttgagtcattgtggttggaattcggtattggaaggtttaagagaaggagtgccaatactcgggtggccgttggcggccgagcaagcgtataattcaaagatgatggtggaggaaatgggggtggcggtcgagttgacgagggggttggaaggggaggtgaagaaagagtgggtgaagagtgtggtggaaatggtgttggatagaaaggaggggagttgtgggtgggaaatgaagaagaaagctgttgagattggggagaaattgaaggatgcttggaaaattgagggagattacaaaggatcttctgttaaggcaatggataattttgtagagtttatagtttcttgtaggggaaagaaatctaaagaagatgcttga

SEQ ID NO: 20 - protein, 491 aa, Chenopodium quinoa
MESSTKEQEQQHIILLPFLAQGHLRPFLHLAHRLLSLTPFNLSLLTTPLNAANLRRQSDNFNINLNIIELPFTSTDHGLPPNTENTDKLSLTSIIKLFHASTSLEPHVRDYLTRHHLHDPPVCIIFDVFLGWADNVARSVGSTGICFNTGGAYGVGAYVSIWSNLPHRNVGDDEEFSLAGFPEDRKLRRNQLHRFLRFADGSDEWSRFFQPQIKSSLNCSGWLCNSIEEIEPLGFQVFRNFTKSPIWGIGPLIMTSSKKDDDEKEEACLRWLNQFENDSVLYICFGSQNTVTPIQMMELAKGLEESKIPFLWVIRPPFGFDFNGEFKPEWLPEKFEERMMEKKQGMLVRDWVPQLDILRHEATGGFLSHCGWNSVLEGLREGVPILGWPLAAEQAYNSKMMVEEMGVAVELTRGLEGEVKKEWVKSVVEMVLDRKEGSCGWEMKKKAVEIGEKLKDAWKIEGDYKGSSVKAMDNFVEFIVSCRGKKSKEDA

SEQ ID NO: 21 - DNA, 1446 bp, unidentified
  source 1..1446: mol_type = genomic DNA
atgagcaaaatagagctggtgttggttccaaccccagggatgggccacttgctctccgccgtcgagctatccaagctcattatccgccgtgagaaccgtatctcagtcctcatcctaatcttaagcttcccctttgactccggtttggttaatgcctacgttgatttccagtcacgtgaccctgacaactccggtagcctaaccttcatcactctcccaccactctccaacatacctgactgcacttcgagcaccttcttcaccaccgtcattgaactccacaagccaaatgtcaagcaggttgtcgaggaacgagtcaggtccgggtcacccaagcctgctgggtttgttatagacatgctttgccctgctatgatggatgtcgcggaggagcttgaggtaccttcttacatattgttcacctctggggctaacttgttgaatgtagtgtttcatttcctgtcactcgctgataatggcgtggatatcgccactgaagttaatgatccggataaggaggtggatgttcccgggttcaggaaccgggtgccctgcaaggtcttacccttgcccttccttgagaaagattttctagtgaagcgcggaaggaggtttagacgatccaacggtattctagtgaacacttccaatgagctggaatcgtatgcgattcagacgttgcttgagcaggctaaggataataagatcccacccgtttatccggtaggtcccattttggagctgaatagcaaaagccgttgtgggactaaggaagatgaggaagtctcgatcatgaggtggctagacgagcagccagtaaactcggtgctgttcgtttgctttggaagcatgggaacattcgatgaggatcaagtaaaggagatagccaatggtctagagcagtcggggtattgctttctatggtccttacgccagccacccccggaagggaaggcgacaccaagcgaggaggccttcctggacacccttccagaggggttcgtagagcgtacatcccataaggggaagatcatcgggtgggctccgcaggtatcaatactagcccacaaggctgttgggggattcgtgtcacattgtggttggaactcgactttagagagtctctggtttggggtcccaatggcaacgtggccaattagcgcggagcagcagctgaacgcatttgagctagtgaaagagttcgggatggcggtagagattcggatggatttttggcgagattgtaggaaaaatactcagagctttgtagtaacgagtgaggagatagagaatggggtgaagaagctgatgagtatggatgaagaaatggtcgagaaagtaaagaagatgagtgataagagtaggaagactttagaggatggcggatcttcgcatcactcgttgggccgtttcattaatgatctcttggagaacgctggcttctga

SEQ ID NO: 22 - protein, 481 aa, unidentified
MSKIELVLVPTPGMGHLLSAVELSKLIIRRENRISVLILILSFPFDSGLVNAYVDFQSRDPDNSGSLTFITLPPLSNIPDCTSSTFFTTVIELHKPNVKQVVEERVRSGSPKPAGFVIDMLCPAMMDVAEELEVPSYILFTSGANLLNVVFHFLSLADNGVDIATEVNDPDKEVDVPGFRNRVPCKVLPLPFLEKDFLVKRGRRFRRSNGILVNTSNELESYAIQTLLEQAKDNKIPPVYPVGPILELNSKSRCGTKEDEEVSIMRWLDEQPVNSVLFVCFGSMGTFDEDQVKEIANGLEQSGYCFLWSLRQPPPEGKATPSEEAFLDTLPEGFVERTSHKGKIIGWAPQVSILAHKAVGGFVSHCGWNSTLESLWFGVPMATWPISAEQQLNAFELVKEFGMAVEIRMDFWRDCRKNTQSFVVTSEEIENGVKKLMSMDEEMVEKVKKMSDKSRKTLEDGGSSHHSLGRFINDLLENAGF

SEQ ID NO: 23 - DNA, 1389 bp, Chenopodium quinoa
  source 1..1389: mol_type = genomic DNA
atgtcacaaaacaaagacaaccaaattctaaacgttacattttacccatggtttgcactaggccaccttacttcttttctccgcttagccaacaaacttgccgaaagaggtcacaatgtctcttattttctcccaccaaaaacacaatcaaaattagcttctcacaaccactacccaacccaccttaccttcatccccatccccgtcccacccgtcgaaggcctccctcctggggccgagacaaccaacgatgtacccgcctcattaggccctctcatcatgaccgccatggacatgacacgtgacactatcgagtctcatctagtccgtctcaaacccgatattgttttttacgacttcacttgttggatgcccgaactaggccgaaaacacgggtttaaagccatgcactatatcacagcatacatagcaagatatgcatatcttgcaccatacaaaaaaataccaggatatcaccctaatgcggatgatctattgacaccaccaccagagtttccgtcacaatcaatcaggatgctcccccaagaggctgagatcattgcgggtgcccttaagacaccattcggtctaggaggactaacactagctgagaggctaggtgtttcgtttcgagaatgcgacgcattcggagttaagacttgtgcggagatggaaggggagtattgtaaattttttgagaaaatatttggtaagccagtgttactagcagggccaatggtacctaaacggccatcttcggaacttgataattattttgatgattggctaaatagttttcgtacgtctagtgttatatattgtgcacttggaagtgaatgcgctctgaatttgaaccaatttcaagagcttgttcttggattggagcttacaggtaggccatttttggcagcgttaaagccaccaatgaattaccaaacaatagaatcagcattgccagaaggacttgcggaaagaataaaaggcaggggactaattcatgggggttgggtgcaacagcagctcatcttacaacatccttcggtaggatgttttattacacattgtggtgcaggatctctatccgaagctatggtaagcgaatgtcaagtcgtgctgatgcctcaagcaatcgaccagttcatcagtgcgcggatgatgagcttagagtggaaggttggggtcgaagttgagaagagaaagaattatggtttgttcaccaaggaggccgtccataaggcggtctccttggtgatggaagaagatagtgaagttgggagagatgtaagggctaatcatgctaaatggagagagtttattttaactgaaggtcttgaggattcttatattagtagcttcatacagagtttgcaacaattgataggatcataa

SEQ ID NO: 24 - protein, 462 aa, Chenopodium quinoa
MSQNKDNQILNVTFYPWFALGHLTSFLRLANKLAERGHNVSYFLPPKTQSKLASHNHYPTHLTFIPIPVPPVEGLPPGAETTNDVPASLGPLIMTAMDMTRDTIESHLVRLKPDIVFYDFTCWMPELGRKHGFKAMHYITAYIARYAYLAPYKKIPGYHPNADDLLTPPPEFPSQSIRMLPQEAEIIAGALKTPFGLGGLTLAERLGVSFRECDAFGVKTCAEMEGEYCKFFEKIFGKPVLLAGPMVPKRPSSELDNYFDDWLNSFRTSSVIYCALGSECALNLNQFQELVLGLELTGRPFLAALKPPMNYQTIESALPEGLAERIKGRGLIHGGWVQQQLILQHPSVGCFITHCGAGSLSEAMVSECQVVLMPQAIDQFISARMMSLEWKVGVEVEKRKNYGLFTKEAVHKAVSLVMEEDSEVGRDVRANHAKWREFILTEGLEDSYISSFIQSLQQLIGS

SEQ ID NO: 25 - DNA, 1389 bp, Chenopodium quinoa
  source 1..1389: mol_type = genomic DNA
atgtcacaaaacaaagacacccaaattctaaacgttgcattttacccatggtttgcactaggccaccttacttcttttctccgcttagccaacaaactagccgaaagaggtcacaaagtctcctattttatcccaccaaaaacacgatcaaagttagcttctcacaaccactacccaacccaccttaccttcatccccatccccgtcccaccggtcgaaggcctccctcccggggccgagacaaccaacgatgtacccgcctcatccggcccactcatcatgaacgccatggacatgacacgtgacaccatcgagacccatctagtccgactcaaacccgatattgttttttacgacttcacttgttggatgcccgaactagcccgaaaacacgggtttaaagcaatacactatatcacagcatacatagcaagatatgcatatcttgcaccatacaaaaaaataccagggtatcaccctaatgcgaatgatctattaacaccaccaccagagtttccgtctcaatcaatcaggatgctcccccaagaggccgagatcatggcgggtgccggtaagacgccatttggtctagggggactaacactggctgagaggctaggtgtttcgtttcgagaatgcgacgcattcggggttaagacttgtacggagatggaaggggagtattgtaagttttttgagaaaatatttggtaagccagtgttactagcagggccaatggtgcctaaaacgccatcttcgaaacttgataagtattttgatgattggttgaatagttttggtaatgctagtgttatatattgtgcacttggaagtgaatgtgctctgaaattgaaccaatttcaagagcttgttcttggactagagcttacaggtaggccatttttggcagccttaaagccaccaatgaattaccaaacaatagaatcagcattaccagaaggatttgcggaaagaacaaaaagcaggggacttattcatggtggttgggtgcaacaacaactcatcgtacaacatccttcggtaggatgttttataacacattgtggtgcaggatctctatccgaagctatggtaagcgaatgtcaagtcgtgttgatgcctcaagcaatcgaccagttcatcagtgcgcgaatgatgagcttagagtggaaggttggggtcgaagttgagaagagaaagaatgatggtttgttcaccaaggaggccgtccataaggcggtctccttggtgatggaagaagacagtgaagttggaagagatgtaagggctaatcatgctaaatggagagagtttatattaactgaaggtcttgaggattcttatattagtagcttcattatgagtttgcaacaattgattggatcatga

SEQ ID NO: 26 - protein, 462 aa, Chenopodium quinoa
MSQNKDTQILNVAFYPWFALGHLTSFLRLANKLAERGHKVSYFIPPKTRSKLASHNHYPTHLTFIPIPVPPVEGLPPGAETTNDVPASSGPLIMNAMDMTRDTIETHLVRLKPDIVFYDFTCWMPELARKHGFKAIHYITAYIARYAYLAPYKKIPGYHPNANDLLTPPPEFPSQSIRMLPQEAEIMAGAGKTPFGLGGLTLAERLGVSFRECDAFGVKTCTEMEGEYCKFFEKIFGKPVLLAGPMVPKTPSSKLDKYFDDWLNSFGNASVIYCALGSECALKLNQFQELVLGLELTGRPFLAALKPPMNYQTIESALPEGFAERTKSRGLIHGGWVQQQLIVQHPSVGCFITHCGAGSLSEAMVSECQVVLMPQAIDQFISARMMSLEWKVGVEVEKRKNDGLFTKEAVHKAVSLVMEEDSEVGRDVRANHAKWREFILTEGLEDSYISSFIMSLQQLIGS

SEQ ID NO: 27 - DNA, 1359 bp, Beta vulgaris
  source 1..1359: mol_type = genomic DNA
atgggtgaaaacaaagaatcgcaagttttgaaggtagcattctacccttggtttgcacttggccaccttacttcattccttcgcttagccaacaaacttgctgaaagaggtcaccaggtctcatattttctgccgaagaatacacaacttaaattagcttctcaaaaccacttcccagaccatctcaccttcatccccatcaccgttccatccgtagacggccttcctcctggggccgagacgaccaacgatgtatccttgtcgggtgcgcaccttatcatggctgccatggacatgactcgtgatactattgatgcccatctggcccttctcaagcccgactttgttttctatgatttcgcttattggatgcctcaactggctcggaaacatgggatcaaatcagtgcactacatcactggattaatagcaagatatactctagctatctcccgtatcacaggggcgtatccacccgatctctcgtccccaatttttcagatgcgtgcccatgagatgaaggttatggagcatataagtgagaagccattcgcaccaggaggtgtgacgttgccggagatgttcaggatatcgttcgaagaatgtgatgctgtcggagtcaagacttgtaaggaaatggaaggaatttactgtgagtttgctgagaagacgttagataagcctgtgcttctcgcagggcctgttgtcccaaaactaccatcttctaaacttgatgactatgttgatgcctggctaagtacttttggttctggtactgttatattttgtgctcttggaagtgaatgtattcttgaacaaaaccagtttcaagatattcttcttggactcgagctcacaggtaagccatttttggcggccctgaagccgcccaagaattgcaaaagtttagagtcgggtttaccagaaggattcagtgagagagtaagaggaagaggaatgatccatggaggttgggtgcagcaacaactgatcttacaacatccatcagtgggatgtttcataactcattgtggggttggatctctatcagaagctatggtaagccaatgtcaagttgtacttatgcctcaagcagtcgatcaattcatgaatgcgaggcagatgagtttagaattgaagatcggagttaaggtcgagtccacagagactgatggttttttcactagggaagccctctgtaaggcggtctcccttgtgatggatgaacaaagtgaagttgcgagagaagtgaaggcaaatcatgctaaatggagagattttattttaaccgaaggtcttgaggactcttatattagtagcttcattcagagtctacaacacttgcttcagatgaactga

SEQ ID NO: 28 - protein, 452 aa, Beta vulgaris
MGENKESQVLKVAFYPWFALGHLTSFLRLANKLAERGHQVSYFLPKNTQLKLASQNHFPDHLTFIPITVPSVDGLPPGAETTNDVSLSGAHLIMAAMDMTRDTIDAHLALLKPDFVFYDFAYWMPQLARKHGIKSVHYITGLIARYTLAISRITGAYPPDLSSPIFQMRAHEMKVMEHISEKPFAPGGVTLPEMFRISFEECDAVGVKTCKEMEGIYCEFAEKTLDKPVLLAGPVVPKLPSSKLDDYVDAWLSTFGSGTVIFCALGSECILEQNQFQDILLGLELTGKPFLAALKPPKNCKSLESGLPEGFSERVRGRGMIHGGWVQQQLILQHPSVGCFITHCGVGSLSEAMVSQCQVVLMPQAVDQFMNARQMSLELKIGVKVESTETDGFFTREALCKAVSLVMDEQSEVAREVKANHAKWRDFILTEGLEDSYISSFIQSLQHLLQMN

SEQ ID NO: 29 - DNA, 1371 bp, Amaranthus hypochondriacus
  source 1..1371: mol_type = genomic DNA
atgggttacaaagaagagtcctctgttatgcatatagcattttacccatggctagctctaggccatataacctccttccttcgtttagccaacaaacttgcacaaaaaggtcatactatctcattcttcatccctaccaaaacccaatccaaattagcttctcaaaaccacttccctaaccacctcaccttcgtccccgtcgatatcccgtctatcgacgggcttcctccaggggccgagaccaccaacgatgtgtcggtctcggccgcaccccttatcatgtccgctatggacatgacccgtgactctatcgaaacccaattagtccatcttaaacccgattttgttttctatgactttgcgtattggatgcctgaattgggtaaaaaacatgggttcaaatccgtgcattacatcacaggttatatagcaagatacgctgcttttgctgcttacattacagcacccgaccatactaacactttagacccaccaccagggctttcttccccaatatttaagatgcaggcccatgagtcccggattctatcagcggtaagtaaacggccgtttgggtcaacaggtaaaacgatgactgaaatgtttggtatatcgtttagggaatgcgatgcgattggagctaaaacttgtacggaaatggaaggggaatattatgagtttgttaaaaagactttaggtaagccattgttgttagccgggcctgttgttcctattcaaccagcatctaagttagatgagaaagttgatgaatggttaaatggttttggagctgaaactgtgatttattgtgcacttggaagtgaatgtgttctagaactttctcagtttcaacagattcttcttggattagagctaacaggtaggccattttttgcagccctaaagccccccaagaattatgaaactatagagtcagccttgccgaaagagttcgaggagagaataaaaggaaaaggaatcattgatagtggttgggttcaacaacaactaatattaaaacatccatcaataggatgttttataacacattgtggagttggatctctgtctgaagctatggtaagccattgtcaagtggtgtttatgcctcaagccgtggaccaattcatcaacgcaaggcaaatgagccttgagttgaagatcggggtggaagttgagtcgcgagaggaggatggtttttacaccaaggaggccgtcagtaaagcggtctccttggtgatggatgaacatagtgaagttggtagagaagtaagagctaaacatgctaaatggagagattttattttaaaacaaggtcttgaggattcttatattactagctttatcaatagtttacgacaattgctttaa

SEQ ID NO: 30 - protein, 456 aa, Amaranthus hypochondriacus
MGYKEESSVMHIAFYPWLALGHITSFLRLANKLAQKGHTISFFIPTKTQSKLASQNHFPNHLTFVPVDIPSIDGLPPGAETTNDVSVSAAPLIMSAMDMTRDSIETQLVHLKPDFVFYDFAYWMPELGKKHGFKSVHYITGYIARYAAFAAYITAPDHTNTLDPPPGLSSPIFKMQAHESRILSAVSKRPFGSTGKTMTEMFGISFRECDAIGAKTCTEMEGEYYEFVKKTLGKPLLLAGPVVPIQPASKLDEKVDEWLNGFGAETVIYCALGSECVLELSQFQQILLGLELTGRPFFAALKPPKNYETIESALPKEFEERIKGKGIIDSGWVQQQLILKHPSIGCFITHCGVGSLSEAMVSHCQVVFMPQAVDQFINARQMSLELKIGVEVESREEDGFYTKEAVSKAVSLVMDEHSEVGREVRAKHAKWRDFILKQGLEDSYITSFINSLRQLL

SEQ ID NO: 31 - DNA, 1386 bp, Amaranthus hypochondriacus
  source 1..1386: mol_type = genomic DNA
atgtctcacaacaaagaatccaacccaaatcttcacgtagcattttacccttggtttgcattaggccacttaacttcattccttcgtttagccaacaaactagccgaaagaggccttcaagtctcttattttatcccatccaaaacccaaccaaaactatcccctcacaaccaccaccctaaccaccttaccttcatacccatcaccgtcccacacgtcgacggcctcccacccggagccgagaccaccaacgatgtccccggctcagccgtacccctcatcatgaccgcaatggacttaacccaagacataattgaggcccatctagcccaactcaaacccaactttgttttctatgacttcacttattggatacctaaactaggccaaaaacttgggtttaaatctatccactattttaccgcatttatatcaagatatggttatcttgcaccttacaaaaaggaaggataccttccaacggcggccgatcttcttcggccgccaccaggctacccatccccaataaggatgaagccttacgaagccaaaattatggcgggcgcaggtaaaaccgcctttggactaggcgggtttacgctggccgagagattagccgtgtcatttatagagtgtgatgcatttggggttaagacttgtaaggagatggaaggtgaacattgtaagttttttgaggatgtttttggtaagccagtgcttttagctggaccagttgtgcctaaacttccatcttctaaactagatgaacattttgatgagtggttaaatgggtttgatgaatctagtgtgattttttgtgcacttggaagtgaatgttctttggaaattaaccaatttcatgagcttcttcttggattagagctcacaggtagaccatttttggcggctctaaagccgccaaaaaattacaaaacaatagaatcagcattaccagaaggttttgcaagcagaactagagaaagaggaatagttcatgaaggttgggtgcaacaacaactaatcttacaacatcgatcagtaggatgttttataactcattgtggagttggttctttatccgaagctatgataagcaaatgtcaagtagtgatgatacctcaagcaattgatcaattcatcaatgcaaggatgatgagtttagagtggaagattggagtcgaaatcgagacgagggaggatgatggttggttcactagggaggatgttcacaaggcaattacaatggtgatggatggagaaagtgatgttgggagagaagttagggctaaccatgctaaatggagggactttattttaacacaaggtgttgaagattcttatattagtagctttatagagagtttacaacaattgttaattgtatag

SEQ ID NO: 32 - protein, 461 aa, Amaranthus hypochondriacus
MSHNKESNPNLHVAFYPWFALGHLTSFLRLANKLAERGLQVSYFIPSKTQPKLSPHNHHPNHLTFIPITVPHVDGLPPGAETTNDVPGSAVPLIMTAMDLTQDIIEAHLAQLKPNFVFYDFTYWIPKLGQKLGFKSIHYFTAFISRYGYLAPYKKEGYLPTAADLLRPPPGYPSPIRMKPYEAKIMAGAGKTAFGLGGFTLAERLAVSFIECDAFGVKTCKEMEGEHCKFFEDVFGKPVLLAGPVVPKLPSSKLDEHFDEWLNGFDESSVIFCALGSECSLEINQFHELLLGLELTGRPFLAALKPPKNYKTIESALPEGFASRTRERGIVHEGWVQQQLILQHRSVGCFITHCGVGSLSEAMISKCQVVMIPQAIDQFINARMMSLEWKIGVEIETREDDGWFTREDVHKAITMVMDGESDVGREVRANHAKWRDFILTQGVEDSYISSFIESLQQLLIV

SEQ ID NO: 33 - DNA, 1263 bp, Arabidopsis thaliana
  source 1..1263: mol_type = genomic DNA
atgggttacaaagaagagtcctctgttatgcacatagcattttacccatggttagctctaggccatataacctccttccttcgtttagccaacaaacttgcacaaaaaggtcacactatctcattcttcatccctaccaaaacccaatccaaattagcttctcaaaaccacttccctaaccacctcaccttcatccccgtcgatatcccgtctatcgacgggcttcctctaggggccgagactaacgatgtgtcggtctcggccgcaccccttatcatgtccgctatggacatgacccgtgaatctatcgaaacccaattagtccatcttaaacccgactttgttttctatgactttgcttattggatgcctgaattgggtaaaaaacatgggttcaaatccgtgcattacatcacaggatatttaacaagatacgctgcttttgctgcttacattacagcacccgatcatactgacattttgggcccaccaccagggctttcttccccaatctttaagacacgggcccacgaggcccgggtcggtctatcagcagtgatcaaaaaaccgtttggattaacaggtaaaacattgattgaaatgtttggtatatcgtttagggaatgcgatgcaattggagcaaatacttgtatggaaatggaaggggaatattatgagtttgtaaacaagactttgggtaagccattgttgttagccgggcctgttgttccaattcaaccagcatctaagttagatgagaaggttaatgaatggttaaatggttttggtgctgaaactgtggtttattgtgcacttggaaagtcagccttgtcagaagagtttaaggagagaataaaagggaaaggaatcattgacggtggttgggttcagcaacaactaatattgaaacatccatcagtaggatgttttgtaacacattgtggaattggatctctatctgaagctatggtaagccattgtcaaatggtgtttatgcctcaagctgtggaccagttcatcaatgcaaggcaaatgagccttgagttgaagatcggggcggaagttgagtcgcgagaggaggatggtttttacaccaaggaggccatcagtaaggcggtctcctcggtgatggatgaacatagtgaagttggtcgagaagtaagagctaaccatgctaaatggagagactttattttaaaacaaggtcttgaggactcttatattattagcttcatcaatagtttacaacaattgcttcattaa

SEQ ID NO: 34 - protein, 420 aa, Arabidopsis thaliana
MGYKEESSVMHIAFYPWLALGHITSFLRLANKLAQKGHTISFFIPTKTQSKLASQNHFPNHLTFIPVDIPSIDGLPLGAETNDVSVSAAPLIMSAMDMTRESIETQLVHLKPDFVFYDFAYWMPELGKKHGFKSVHYITGYLTRYAAFAAYITAPDHTDILGPPPGLSSPIFKTRAHEARVGLSAVIKKPFGLTGKTLIEMFGISFRECDAIGANTCMEMEGEYYEFVNKTLGKPLLLAGPVVPIQPASKLDEKVNEWLNGFGAETVVYCALGKSALSEEFKERIKGKGIIDGGWVQQQLILKHPSVGCFVTHCGIGSLSEAMVSHCQMVFMPQAVDQFINARQMSLELKIGAEVESREEDGFYTKEAISKAVSSVMDEHSEVGREVRANHAKWRDFILKQGLEDSYIISFINSLQQLLH

SEQ ID NO: 35 - protein, 272 aa, Chenopodium quinoa
MKMGGENNNIINETFFITHGNPILTVEDTHPLRPFFETWTKKIFSKKPKAILVISGHWETDHPAVNAVHLNDTIYDFDDYPQAMYKLKYPAPGSPDLASRIEEILKKSGFDTVHIDKKRGLDHGAWVPLMYMYPEADIPVCQLSVQPKMDGTYHYNLGRALAPLKDEGVLIIGSGSATHPLDETPHYHGGVAPWAADFDSWLDLALTKGRFEEVNTYETKAPNWELAHPFPEHFYPLHVVVGAAGEKWKAELIHTSWDHGTLCHASYKFTST

SEQ ID NO: 36 - DNA, 819 bp, Chenopodium quinoa
  source 1..819: mol_type = genomic DNA
atgaaaatgggtggggaaaataacaatataatcaatgaaaccttcttcataacccatggaaacccgattttgaccgtagaagacacacatcccttaaggccattctttgaaacttggactaaaaaaatattttcgaagaagccgaaggcgatacttgtgatctccggtcactgggaaactgatcatcctgctgttaatgctgttcatcttaatgatactatctacgattttgatgactatcctcaagcaatgtacaagttgaagtatccagcaccagggtcaccagacttggctagcagaatagaagaaattctgaaaaagtccgggttcgacacggtgcacattgacaaaaaacgtgggcttgatcatggtgcatgggtgcctcttatgtacatgtatccggaggctgacattccggtgtgtcagctctccgtgcagccgaaaatggacggaacgtaccactacaacttgggacgggcgttggctccgctgaaagacgaaggtgtactcattattggttccggaagtgcaacacacccattggacgaaacccctcattatcatggtggtgttgctccttgggctgctgactttgactcatggcttgacctagctctcactaaaggaaggtttgaagaagtgaatacatatgaaaccaaagcaccaaactgggaattggcacatccattcccggagcatttttatccattgcacgtcgtcgttggggcggctggtgaaaagtggaaggctgagcttattcatactagttgggaccatggtaccttatgtcatgcctcctacaagtttacttccacctaa

SEQ ID NO: 37 - protein, 276 aa, Chenopodium quinoa
MKMVVEEGKNNEMKFNETFFITHGNPILTVEDTHPLRPFFETWTEKIFSKKPKAILVISGHWETDHPAVNAVHINDTIYDFDDYPEAMYKFKYPAPGSLELAKGVQQLLNKSKFETVHIDQKRGLDHGVWVPLMYMYPEADIPVCQLSVQPKMDGTYHYNLGRALAPLRDEGILIIGSGSATHPLDETPHYHGGVAPWAAEFDSWLDTALTNGRIEEVNTYETKAPNWELAHPFPEHFYPLHVVVGAAGEKWKAELIHTSWDHGTLCHGAYKFTSS

SEQ ID NO: 38 - DNA, 831 bp, Chenopodium quinoa
  source 1..831: mol_type = genomic DNA
atgaaaatggttgttgaagagggaaaaaataacgaaatgaaattcaatgaaaccttcttcataacccatggaaacccaattttgacagtagaagacacacatcccttaaggccattctttgaaacttggactgaaaaaatattttcgaagaagccgaaggcgatacttgtgatctccggtcactgggaaactgatcatcctgctgttaatgctgttcatatcaatgatactatctacgattttgatgactatcctgaagccatgtacaagtttaagtatccggctcccgggtctctagaattggcaaaaggggtgcaacaactactcaacaaatccaagttcgaaaccgtgcacattgaccaaaaacgtgggcttgatcatggtgtatgggtgcctcttatgtacatgtatccggaggccgacatcccggtatgtcagctctccgttcagccgaaaatggacggaacataccactacaacttgggacgtgcgttagcgcctctaagagacgaaggtatactcatcattggttccggaagtgcaacacaccccttagacgaaacgcctcattatcatggtggtgttgctccttgggctgccgagtttgattcttggctcgacactgctctcactaatggaaggattgaagaagtgaatacatatgaaaccaaagctccaaactgggaattagcacatccgttcccggagcatttttatccattgcacgtcgtcgttggggcggctggtgaaaagtggaaggctgagcttattcatactagttgggaccatggtaccctatgtcatggcgcttacaagttcacttccagttaa

SEQ ID NO: 39 - protein, 270 aa, Chenopodium quinoa
MGGEDKNIINETFFITHGNPILTVEDTHPLRPFFETWTEKIFSKKPKAILVISGHWETDHPAVNAVHINDTIYDFDDYPEAMYKVKYPAPGSPDLASRVEEILKKSGFDTVHIDKKRGLDHGAWVPLMYMYPEADIPVCQLSVQPKMDGTYHYNLGRALAPLKDEGVLIIGSGSATHPLDETPHYHGGVAPWAADFDSWLDVALTKGRFEEVNTYETKAPNWELAHPFPEHFYPLHVVVGAAGEKWKAELIHTSWDHGTLCHASYKFTST

SEQ ID NO: 40 - DNA, 813 bp, Chenopodium quinoa
  source 1..813: mol_type = genomic DNA
atgggtggggaagataagaatataatcaatgaaaccttcttcataacccatggaaacccaattttaacagtagaagacacgcatcccttaaggccattctttgaaacttggactgaaaaaatattttcgaagaagccgaaggcgatacttgtgatctccggtcactgggaaactgatcatcctgctgttaatgctgttcatatcaatgatactatctacgattttgatgattatcctgaagccatgtacaaagtgaagtatccagctccagggtcaccagacttggctagccgagtagaagaaattctgaaaaagtccgggttcgacacggtgcacattgacaaaaaacgtgggcttgatcatggtgcatgggtgcctcttatgtacatgtatccggaggctgacattccggtatgtcagctctccgttcagccgaaaatggacggaacgtaccactacaacttggggcgggcgttggcgccactgaaagacgaaggtgttctcattattggctccggaagtgcaacacaccctttggacgaaacccctcattatcatggtggtgttgctccttgggctgctgactttgactcgtggcttgacgttgctctcactaaaggaaggtttgaagaagtgaatacatatgaaaccaaagcaccaaactgggaattggcacatccattcccggagcatttttatccattgcacgtcgtcgtcggggcggctggtgaaaagtggaaggctgagcttattcatactagttgggaccatggtaccttatgtcatgcctcctacaagtttacttccacctaa

SEQ ID NO: 41 - protein, 270 aa, Chenopodium quinoa
MGGEDNNMIKETFFITHGNPILTVVDTHPLRPFFETWSEKIFLKKPKAILVISGHWETNHPAVNAVHINDVIYDFDDYPEAMYQLKYPAPGSPELASSVEENLKKSGFDTVHIDKKRGLDHGAWVPLMYMYPEADIPVCQLSVQPKLDGTYHYNLGRALAPLKDEGVLIIGSGSATHPLDETPHYHGGVAPWAADFDSWLDVALTKGRFEEVNTYETKAPNWELAHPFPENFYPLHVVIGAAGEMWKAELIHNSWDHGTLCHGSYKFTST

SEQ ID NO: 42 - DNA, 813 bp, Chenopodium quinoa
  source 1..813: mol_type = genomic DNA
atgggaggagaagataacaatatgatcaaagaaaccttcttcataacccatggaaacccgattttgaccgtggtagacactcatcccttaaggccattctttgaaacatggagtgaaaaaatatttttgaaaaagccgaaggcgatacttgtgatctctggtcactgggaaactaatcatcctgctgttaatgctgttcatatcaatgatgttatctatgatttcgatgactatcctgaagccatgtaccagttgaaatatccagctccagggtcaccagagttggctagcagtgtagaagaaaatctgaaaaaatccgggttcgacacggtgcacattgacaaaaaacgtgggcttgatcatggtgcatgggtgcctcttatgtacatgtatccggaggctgacattccggtatgtcagctctccgttcagccaaaattggacggaacgtaccactacaacttgggacgggcgttggcgccgctgaaagacgaaggtgtactcatcattggttccggaagcgcaacacaccctttggacgaaacccctcattatcatggtggtgttgctccttgggctgctgactttgattcttggcttgacgtagctctcactaaaggaaggtttgaagaagtgaatacatatgaaaccaaagcaccaaactgggaattggcacatccattcccggagaatttttatccattgcatgttgtcattggggcggctggtgaaatgtggaaggctgagcttattcataatagttgggaccatggtaccttatgtcatggctcctacaagtttacttccacctaa

SEQ ID NO: 43 - protein, 272 aa, Chenopodium quinoa
MKMSGEDNSMIKETFFITHGNPILTVEDTHPLRPFFETWREKIFTMKPKAILVISGHWETDHPAVNAVHINDTIYDFDDYPQAMYKLKYPAPGSPDLASRVEEIMKKSGFDTVHIDKKRGLDHGAWVPLMYMYPEADIPVCQLSVQPKLDGTYHYNLGRALAPLRDEGVLIIGSGSATHPLDETPHYHGGVAPWAADFDSWLDVALTKGRFEEVNTYETKAPNWELAHPFPEHFYPLHVVVGAAGEKWKAELIHTSWDHGTLCHGSYKFTST

SEQ ID NO: 44 - DNA, 819 bp, Chenopodium quinoa
  source 1..819: mol_type = genomic DNA
atgaaaatgagtggagaagataatagcatgatcaaagaaactttcttcataacccatggaaacccgattttgaccgtagaagacacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaa

SEQ ID NO: 45 - protein, 276 aa, Chenopodium quinoa
MKMIIEEEKNNEMNFNETFFITHGNPILTVEDTHPLRPFFETWKEKVFSKKPKAILVISGHWETDHPAVNAVHVNDTICDFDDYPQAMYKFKYPAPGYLELAKRVQQLLNKSKFETVHIDQKRGLDHGAWVPLMYMYPEADIPVCQLSVQPKLDGTYHYNLGRALAPLRDESVLIIGSGSATHPLDETPHYHGGVAPWAAEFDSWLDTALTNGRIEEVNTYETKAPNWELAHPFPEHFYPLHVVVGAAGEKWKAELIHTSWDHGTLCHGAYKFTSS

SEQ ID NO: 46 - DNA, 831 bp, Chenopodium quinoa
  source 1..831: mol_type = genomic DNA
atgaaaatgattattgaagaggaaaaaaataacgaaatgaatttcaatgaaaccttcttcataacccatggaaacccgattttgacagtagaagacacacatcccttaaggccattctttgaaacatggaaagaaaaagtattttcgaagaagccgaaggcgatacttgtgatctctggtcactgggaaactgatcatcctgctgttaatgctgttcatgtcaatgatactatctgcgattttgatgactatcctcaagccatgtacaagtttaagtatccggctcccgggtatctagaattggcaaaaagggtgcagcaactactcaataaatccaagttcgaaaccgtgcacattgaccaaaaacgtgggcttgatcatggtgcatgggtgcctcttatgtacatgtatccggaggccgacatcccggtatgtcagctctccgttcagccaaaactggacggaacataccactacaacttgggacgagcattagcgcctctaagagacgaaagtgtactcatcattggttccggaagtgcaacacaccccttagacgaaacccctcattatcatggtggtgttgctccttgggctgccgagtttgattcttggcttgacactgctctcactaatggaaggattgaagaagtgaatacatatgaaaccaaagctccaaactgggaattagcacatccattcccagaacatttttatccattgcacgttgtcgtcggggcagctggtgaaaagtggaaggctgagcttattcataccagttgggaccatggtaccctatgtcatggcgcatacaagttcacttccagttaa

SEQ ID NO: 47 - protein, 267 aa, Mirabilis jalapa
MKGTYYINHGDPLMYLKKHIKLRQFLEGWQENVVIEKPKSILIISAHWDTNVPTVNFVEHCDTIHDFDDYPDPLYQIQYRAPGAPNLAKKVEELLKESGMECEIDTKRGLDHAAWFPLMFMYPEANIPICELSVQPSKDGIHHYNVGKALSPLLQQGVLIIGSGGTVHPSDDTPHCPNGVAPWAIEFDNWLEDALLSGRYEDVNNFKKLAPNWEISHPGQEHLYPLHVALGAAGKNPKTQLIHRSWAANGVFGYSTYNFTPTTQKTD

SEQ ID NO: 48 - DNA, 804 bp, Mirabilis jalapa
  source 1..804: mol_type = genomic DNA
atgaaaggaacatactatataaatcatggtgatccactaatgtacttaaaaaaacatataaaactaaggcaattcttagaaggatggcaagaaaatgttgttattgaaaaaccaaagagtatacttatcatttctgctcattgggatactaatgtacctactgtcaactttgttgaacattgtgatactattcatgattttgatgactatcctgatcctttgtaccagatacaatatcgagcaccgggagcaccaaatttagcaaaaaaggtggaagagttactaaaagagtcaggaatggaatgtgagatagatacaaagagaggactcgatcatgcagcatggtttccactaatgtttatgtatcctgaagctaatattcctatttgtgagctctcagtccaaccaagcaaagatgggatccaccactataatgttgggaaagctctttctcctcttttgcaacaaggtgttctcatcattggctctggtggtactgttcatccttctgatgatactcctcattgtcccaatggtgttgctccttgggctattgagtttgataactggcttgaagatgctcttcttagtggaaggtatgaagatgtgaacaacttcaagaaattggcaccaaattgggagatatctcatccaggacaagaacatttgtaccctttgcatgtggcattaggggctgctggcaaaaatccaaagacacaacttattcatcgaagctgggctgccaatggtgtctttggatattccacctacaacttcactcccaccactcaaaaaactgattaa

SEQ ID NO: 49 - protein, 48 aa, Chenopodium quinoa
  VARIANT 19: note = Xaa stands for Asp or Glu
  VARIANT 26: note = Xaa stands for Leu, Val or Thr
  VARIANT 30: note = Xaa stands for Lys or Asn
  VARIANT 33: note = Xaa stands for Phe or Ile
HPLDETPHYHGGVAPWAAXFDSWLDXALTXGRXEEVNTYETKAPNWEL

SEQ ID NO: 50 - DNA, 1515 bp, Chenopodium quinoa
  source 1..1515: mol_type = genomic DNA
atggaacatacaacacttgcattgatactttcaattttgtttatttgctttcatttagtccgatcatttgttagtcactcaacaaagtccaataaacttccacccgggcctaaaagaatgccaatttttggcaatattttggatcttggggagaagcctcatcgatcatttgcaaatcttgctaagattcacggccctttggtgagtttgcagttaggaagtatcacaacaattatagtgtcttcagctgatgtagccaaagaaatgtttcttaaaaacgaccagttacttgctaaccgaaccattcctgattctgttagggcaggaaaccacgacaagctctccatgtcgtggttgcccatatcagctaaatggaggaatctcagaaagatctccgccgtgcaacttctctctaatcaacgacttgatgctagccaagcccatagacaagccaaggtcgaacaacttcttgcatacgtgcaagattgttctaagaaagggcaacccgttgatatagggagggccgcgtttacaacttctctaaacttattatcaaacacatttttctcaacggaattggcaagccacgagtcgaataattcccaagagtttaagcaactcatgtggaacataatggaggaaattggtagaccaaattatgccgattatttccccattcttggctacgttgatccgttcggcataaggcgtcgtttggctgcttactttgataaactcatagctgtttttcaagacataatttgtgaaaggcaaaaaattcgatcaaccaaagtttcaagtgaaaaacaaacgggtgatattttagacacacttctcaacctttatgatgaaaatgagttgagtatgggtgagattaatcatcttcttgtggacatctttgatgccggaacagatactacagctagcacattagagtgggcaatggctgagcttgttaaaaatcccgggatgatgataagagtacaaaatgaaatcgaactagccatcggcaaaggttgctccatggtccaagaagctgatatctcaaaactaccttacttgcaagccatcatcaaagaaacattgcgattgcaccctccaactgtatttctcttacctcgaaaggcagacattgatgtagagttatatggttacgtcgtaccaaaaaatgcacaagttctagtcaatctttgggccattggtcgcgatccaaaggtatggaaaaatcccgaaatattttcacctgaaaggtttttagggtgtgacatcgattttaaaggacgagattttgaactcttgccctttggtgccggaagaaggatatgccctggattaactttggcttatagaatgttgaacttaatgatggctaattttgttcattcctttgattggaagcttgaagatggaatgaatccaaaagatttggacatggatgagaaatttggtatcacattgcaaaaggttaagcctctccaagttattccagtacataggaaacactgcatgtcataa

SEQ ID NO: 51 - protein, 504 aa, Chenopodium quinoa
MEHTTLALILSILFICFHLVRSFVSHSTKSNKLPPGPKRMPIFGNILDLGEKPHRSFANLAKIHGPLVSLQLGSITTIIVSSADVAKEMFLKNDQLLANRTIPDSVRAGNHDKLSMSWLPISAKWRNLRKISAVQLLSNQRLDASQAHRQAKVEQLLAYVQDCSKKGQPVDIGRAAFTTSLNLLSNTFFSTELASHESNNSQEFKQLMWNIMEEIGRPNYADYFPILGYVDPFGIRRRLAAYFDKLIAVFQDIICERQKIRSTKVSSEKQTGDILDTLLNLYDENELSMGEINHLLVDIFDAGTDTTASTLEWAMAELVKNPGMMIRVQNEIELAIGKGCSMVQEADISKLPYLQAIIKETLRLHPPTVFLLPRKADIDVELYGYVVPKNAQVLVNLWAIGRDPKVWKNPEIFSPERFLGCDIDFKGRDFELLPFGAGRRICPGLTLAYRMLNLMMANFVHSFDWKLEDGMNPKDLDMDEKFGITLQKVKPLQVIPVHRKHCMS

SEQ ID NO: 52 - DNA, 1518 bp, Chenopodium quinoa
  source 1..1518: mol_type = genomic DNA
atggaacatacaatacttgcattgatactttcaattttgtttatttgctttcatttggttcgatcatttgttagccactctacaaagtccaataaactcccacccgggcctaaaagaatgccaatctttggcaatattttcgatcttggggagaagcctcatcgatcatttgcaaatcttgctaagattcacgggcctttggtgagcttacagttaggaagtatcacaactattgtagtgtcttcagctgatgtagccaaagaaatgtttcttaaaaatgaccaggcacttgctaatcgaaccattcctgattctgttagggcaggagaccatgacaagctctctatgtcgtggttgcccatatcagctaaatggaggaatcttagaaagatctccgccgtgcaacttctctctaatcaacgacttgatgctagccaagcccatagacaagccaaggtcgaacaacttcttgcatacgtgcaagattgttctaagaaaggccaacccgttgatatagggagggccgcgtttacaacttctctaaacttgttatcaaacacatttttttcaacggaattggcaagccatgagtctaataattcacaagagtttaagcaactcatgtggaacataatggaggaaattggtaaaccaaattacgccgatttttttcccattcttggctacgttgatccattcggaataaggcgtcgtttggctggttactttgataaactcattgctgtttttcaagacataatttgtgaaagacaaaaaattcgatcaaccaaagtttcaagtgaaaaacaaacgggtgatattttagacacacttctcaacctctacgatgaaaatgagttgagtatgggtgagattaatcatcttcttgtggacatctttgatgccggaacagatactacagctagcacattagagtgggcaatggctgagcttgttaaaaatcctgggatgatgattagagttcaaaatgaaatcgaactagccattggcaaaggttgctccatggtccaagaatctgatatctcaaaactaccttacttgcaagccatcattaaagaaacattgcgattgcaccctccaaccgtatttctcttacctcgaaaagcagacgttgatgtagagttatatggttacgtcgtacccaaaaatgcacaagttctagtaaatctttgggccattggtcgcgatccaaaggtatggaaaaatccagaaatattttcacctgaaaggtttatagggtgtgacattgattttaagggacgagattttgaactcttgccctttggtgccggaagaaggatatgccctggattaactttggcttatagaatgttgaacttaatgatggcttattttgttcattcctttgattggaaacttgaagatggaatgaatccaaaagacttggacatggatgagaaatttggtatcactttgcaaaaggttaaacctctacaagttattccagtacaaaggaaacaatattgcatgtcataa

SEQ ID NO: 53 - protein, 505 aa, Chenopodium quinoa
MEHTILALILSILFICFHLVRSFVSHSTKSNKLPPGPKRMPIFGNIFDLGEKPHRSFANLAKIHGPLVSLQLGSITTIVVSSADVAKEMFLKNDQALANRTIPDSVRAGDHDKLSMSWLPISAKWRNLRKISAVQLLSNQRLDASQAHRQAKVEQLLAYVQDCSKKGQPVDIGRAAFTTSLNLLSNTFFSTELASHESNNSQEFKQLMWNIMEEIGKPNYADFFPILGYVDPFGIRRRLAGYFDKLIAVFQDIICERQKIRSTKVSSEKQTGDILDTLLNLYDENELSMGEINHLLVDIFDAGTDTTASTLEWAMAELVKNPGMMIRVQNEIELAIGKGCSMVQESDISKLPYLQAIIKETLRLHPPTVFLLPRKADVDVELYGYVVPKNAQVLVNLWAIGRDPKVWKNPEIFSPERFIGCDIDFKGRDFELLPFGAGRRICPGLTLAYRMLNLMMAYFVHSFDWKLEDGMNPKDLDMDEKFGITLQKVKPLQVIPVQRKQYCMS

SEQ ID NO: 54 - DNA, 1503 bp, Chenopodium quinoa
  source 1..1503: mol_type = genomic DNA
atggatagtacaacacttgttatggttgttatttctattttatttgtatttttatatcatgtcaaatccttttttatccgttatttttctaacaggctcccaccagggccaaaaccaaagccaatttttggcaacattttcgatcttggagaaaagcctcatcgatcctttgccaacctagcaaaaatttttggcccattgattagtctaaagctaggcaatgtcacaacagttgttgtatcatcctcttatgtggcagaagagatgtttctaaaaaatgaccagtcatttgcaaatcgaaccattcctgattctgttcgagctggcaaccatgacaaactctccatgtcatggttgcctatatctccacaatggcggaatcttaggaagatctctgctgtccaattactctcaacacaacgccttgatgctagccaagccctaaggcaagccaaggtatcacaacttcatgcatacgtgcaagattgctctaaaaaattgcaaccggttaacataggaagggcagcattcactacttccctaaacttattatcaaacacatttttctcaatcgaactagctagccacgagtctagtacttcccaagagtttaagcaactcatgtggaacataatggaggaaattggaagacctaattatgcagattatttccccattcttggctacattgatccatttggcataagacgtcgtttggcttcttactttgatgaactcattgttgtttttcaaaacattatttgcgaaaggcaaaacattcgatcaagtgacgattcaagtgcaaaacatacaaatgatgttctagacacacttctcaacctctatgacaagaatgagttgagtatggatgaaattaatcatcttcttgttgatatttttaacgcgggaacagacactactgcaagtactttagaatggacaatgacagagctaattaaaaatccaaaaagtatgatcatatgtcaaaatgaaattgaacaagctcttggaaaaggtagtttgagcattcaagaatctgatatttcaaaattgccttacctacaagccatcatgaaagaaacgttacgtttgcaccctcctacagtgtttctcctcccacgtaaagcagacagtgatgtagagttatgtggctacgtagttccaaaacatgcacaagttcttgttaatctatgggctattggtcgtgatccaaaggtgtgggaaaaccctgaagtattttcaccggagaggttcttagattgtgagatcgatttgaaaggacaagattttgaacttttaccctttggagctggtagaagaatatgccctggacttaatttggcatatagaatgttgaacttgatgttggctactcttcttcacaactacaattggaaacttgaagatggtatgactcttaatgatttagacatggatgaaaagtttggaattacattgcagaaggttaagcctcttcaagttgttccaatcccaaggtattaa

SEQ ID NO: 55 - protein, 500 aa, Chenopodium quinoa
MDSTTLVMVVISILFVFLYHVKSFFIRYFSNRLPPGPKPKPIFGNIFDLGEKPHRSFANLAKIFGPLISLKLGNVTTVVVSSSYVAEEMFLKNDQSFANRTIPDSVRAGNHDKLSMSWLPISPQWRNLRKISAVQLLSTQRLDASQALRQAKVSQLHAYVQDCSKKLQPVNIGRAAFTTSLNLLSNTFFSIELASHESSTSQEFKQLMWNIMEEIGRPNYADYFPILGYIDPFGIRRRLASYFDELIVVFQNIICERQNIRSSDDSSAKHTNDVLDTLLNLYDKNELSMDEINHLLVDIFNAGTDTTASTLEWTMTELIKNPKSMIICQNEIEQALGKGSLSIQESDISKLPYLQAIMKETLRLHPPTVFLLPRKADSDVELCGYVVPKHAQVLVNLWAIGRDPKVWENPEVFSPERFLDCEIDLKGQDFELLPFGAGRRICPGLNLAYRMLNLMLATLLHNYNWKLEDGMTLNDLDMDEKFGITLQKVKPLQVVPIPRY

SEQ ID NO: 56 - DNA, 1431 bp, Chenopodium quinoa
  source 1..1431: mol_type = genomic DNA
atgagcaaaagggttttatccggtgatgtttggtggtggttgacggtggttttggtggttgttgttggtggttatgatggcgtaaactcgtacccgaaagaggaccttgttgtaaatttacctggacaaccaaaggttgatttcaggcaatttgctgggtacattgatgttgatgttaaagcaggaagaagcttgttttattattttgttgaagctgaaaataatactcaacatactcctcttactctttggcttaatggaggacctggttgttcatcaatgggaggtggtgcatttacagaattgggaccattttttccaaagggtgatggaagaggtttaagggttaattcaaagtcatggaatagagcgtcgaatctactctttgttgaatcgcctgctggagtgggatggtcgtattcaaacaaatcgtctgattataataccggggatgtagcctctgctaaggatatgctcacatttctggtgaattggatggtgaagtttcctgaattcaaatccagggatctgtatcttactggggagagttatgcagggcactacataccacaactagctgttgctgtgttggatcataatgctcaatctaaggcctcaaagcttaacttaaaaggagttgctattggaaatccacttctcaaactcgatagagatgttccagcaacatatgagtttttctggtctcatggaatgatatctgatgaaattggccttgccatcatgaatcaatgtgattttgaagattatacctttccaaatcctcataacgaaactgatgcttgcaacaatgcaatatcaaaagcaaataccattgttggtgattatgtcaatgcatatgatgtcatccttgatgtctgttatccatctatcgtagagcaagaactgcggttgaaaaaaatggccactaaaatgagtgttggagttgatgtttgtattacttatgaaagaagattctatttcaatcttcctgaagtacagagagctctccatgcaaatcggacaaatctaccttatgaatggtctatgtgcagtagcattctgaattactctgacactgatggtagcttgaatatgcttcctctgcttaaaagaatactgttgcacgacattcgtgtttggattttcagtggagaccaagattctgttgtgcccctgcttggctcccgtacccttgttcgtgagcttgctcatgacctcaaatacaacgtcaccgtcccatatgcaaactggtttcacaaaggacaggtaggaggttggacaaccgaatatggaaacttactcacatttgcgactgtgaggggcgctgcacatatggtaccttatgcacaaccatcaagggctctgcatctgttcagttcatttattcgtgatcgaagactgccaacaacaactaaagaatccgttgaatga

SEQ ID NO: 57 - protein, 476 aa, Chenopodium quinoa
MSKRVLSGDVWWWLTVVLVVVVGGYDGVNSYPKEDLVVNLPGQPKVDFRQFAGYIDVDVKAGRSLFYYFVEAENNTQHTPLTLWLNGGPGCSSMGGGAFTELGPFFPKGDGRGLRVNSKSWNRASNLLFVESPAGVGWSYSNKSSDYNTGDVASAKDMLTFLVNWMVKFPEFKSRDLYLTGESYAGHYIPQLAVAVLDHNAQSKASKLNLKGVAIGNPLLKLDRDVPATYEFFWSHGMISDEIGLAIMNQCDFEDYTFPNPHNETDACNNAISKANTIVGDYVNAYDVILDVCYPSIVEQELRLKKMATKMSVGVDVCITYERRFYFNLPEVQRALHANRTNLPYEWSMCSSILNYSDTDGSLNMLPLLKRILLHDIRVWIFSGDQDSVVPLLGSRTLVRELAHDLKYNVTVPYANWFHKGQVGGWTTEYGNLLTFATVRGAAHMVPYAQPSRALHLFSSFIRDRRLPTTTKESVE

SEQ ID NO: 58 - DNA, 1431 bp, Chenopodium quinoa
  source 1..1431: mol_type = genomic DNA
atgagcaaaagggttttatccggtggtttttgttggtggttgacggtggttttggtggttgttgttggtggttttgattgcgtaacggcgtacccgaaagaggatcttgttgtaaatttacctggacaaccaaaggttgatttcaagcaatttgctgggtacattgatgttgatgttaaagcaggaagaagcttgttttattattttgttgaagctgaaaataatactcaacatactcctcttagtctttggcttaatggaggacctggttgttcatcaatgggaggtggtgcatttacagaattgggaccattttttccaaggggtgatggaagaggtttaagggttaattcaaagtcatggaatagagcgtcgaatctactctttgttgaatcgcctgctggtgtgggatggtcgtattcaaacacatcgtctgattataataccggggatgcaacctctgctaaggatatgctcaaatttctggtgaattggatggtcaagtttcctgaatttaaatccagggatctgtatcttactggggagagttatgcagggcactacataccacaactggctgttgctatattggatcataatgctcaatcaaaggcctccaagctcaacttgaaaggagttgctattggaaatccacttctcaaactcgatagagatgttcctgcaacatacgagttttactggtctcatggaatgatctctgatgaaattggccttgccatcatgaatcagtgtgattttgaagattatacctttccaaatcctcataacgaaactgatgtttgcaacaatgctatatcaaaagcaaataccattgttggcgattatgtcaacgagtatgatgttatccttgatgtctgttatccatctattgtagagcaagaactgcggttgaaaaaaatggccactaaaatgagtgttggagttgatgtttgtatgacttatgaaagacgattctatttcaatcttcctgaagtacagagagctctccacgcaaatcggactaatctaccttatgaatggtctatgtgcagtagcattctgaattacactgacactgatggtagcttgaatatgcttcctctgctgaaaaggatactgttgcacaatattcgtgtatggatcttcagtggagatcaagattctgttgtgccactgcttggctcccgtacccttgttcgtgagcttgctcatgacctcaaatacaatatcaccgtcccctatgcaaactggtttcacaaaggacaggtaggaggttggactactgaatatggaaacttactcacatttgcgactgtgaggggcgctgcacatatggtaccttatgcacaaccatcaagggctctgcacctgttcagttcatttatacatgatcgaaaactgccaacaacaactaaagaatccatagaatga

SEQ ID NO: 59 - protein, 476 aa, Chenopodium quinoa
MSKRVLSGGFCWWLTVVLVVVVGGFDCVTAYPKEDLVVNLPGQPKVDFKQFAGYIDVDVKAGRSLFYYFVEAENNTQHTPLSLWLNGGPGCSSMGGGAFTELGPFFPRGDGRGLRVNSKSWNRASNLLFVESPAGVGWSYSNTSSDYNTGDATSAKDMLKFLVNWMVKFPEFKSRDLYLTGESYAGHYIPQLAVAILDHNAQSKASKLNLKGVAIGNPLLKLDRDVPATYEFYWSHGMISDEIGLAIMNQCDFEDYTFPNPHNETDVCNNAISKANTIVGDYVNEYDVILDVCYPSIVEQELRLKKMATKMSVGVDVCMTYERRFYFNLPEVQRALHANRTNLPYEWSMCSSILNYTDTDGSLNMLPLLKRILLHNIRVWIFSGDQDSVVPLLGSRTLVRELAHDLKYNITVPYANWFHKGQVGGWTTEYGNLLTFATVRGAAHMVPYAQPSRALHLFSSFIHDRKLPTTTKESIE

SEQ ID NO: 60 - DNA, 1494 bp, Chenopodium quinoa
  source 1..1494: mol_type = genomic DNA
atggaaaagttgatcattttcttgacagtcctcttcttcctctcttccttcacctctgccaaaatttcggttttcaacgaatttcaggctgataatcccattgaagcgttcaatttattcccaaaaagacccgttaacataatcaatagctctgataatggcgatctttctcacagcagctcaaaaatcgtcgaaaagaggttcaaatttcccaatttctacgatcctagtggtgcaactgttgaagaactgggccatcatgctggttattatcagattcaacactctcataatgctaggatgttctactttttctttgaatcacgtaacagaaaagaagaccctgttgttatctggttaactggagggcctggttgcagcagtgagttggctctcttttatgaaaatgggccatttaaaattgcaaacaacttgtcccttgtatggaatgaatatggctgggaccaggctgccaacctcatctttgttgaccagcccattgggactggattcagttactccactgatagacatgacatacgacatgatgaaagtggggttagcaatgatttgtatgactttttacaggcattctttaccaagcacccaagtttcgcaaaaaatgacttctacatcactggagaatcatatgctggccactacattcctgcctttgctgcttgtgttcatagcaagaataaagctaagaaaggaattcatatcaacctcaagggatttgctattggcaatgggctgactgatcctgcagtgcagtatgaagcatatcctgattatgcattagagatggggttgatcaagaaatcatcctacaatgtgatcactaagatggttcctctgtgtgaaacagcagtaaaactctgtggtacaaagggtacagtctcctgcatgacttcataccttttatgcaacactatatttacaagcattagactgctaattggtgatactaattattacgatataagaaaaaagtgtgagggacgcttgtgctatgacttctcaaacatggaaaagttcttaaatcaggaatctgttaggagtgctctgggtgttggagacattgaatttcagtcttgtagcaccacggtatatcaggccttgcttgttgattggatgagaaatcttgaggttggcatcccagctcttcttgaggatggaattcagttgcttgtatatgctggagaatacgatctcatctgcaattggcttggcaactcaagatgggtccatgctatggaatggagtgcgcagcaagattttgtagcatcacctgagattcctttcgaagttgatggctctgaggcaggaatactgaagagccttggtcctttgagtttcctcaaggttcatgatgctggtcacatggttcctatggatcagccgaaggcagccttagaaatgttgagacgatggactaaaggctcacttttgctatctacttctacgcctcaaacttttgttgcttcccagtga

SEQ ID NO: 61 - protein, 497 aa, Chenopodium quinoa
MEKLIIFLTVLFFLSSFTSAKISVFNEFQADNPIEAFNLFPKRPVNIINSSDNGDLSHSSSKIVEKRFKFPNFYDPSGATVEELGHHAGYYQIQHSHNARMFYFFFESRNRKEDPVVIWLTGGPGCSSELALFYENGPFKIANNLSLVWNEYGWDQAANLIFVDQPIGTGFSYSTDRHDIRHDESGVSNDLYDFLQAFFTKHPSFAKNDFYITGESYAGHYIPAFAACVHSKNKAKKGIHINLKGFAIGNGLTDPAVQYEAYPDYALEMGLIKKSSYNVITKMVPLCETAVKLCGTKGTVSCMTSYLLCNTIFTSIRLLIGDTNYYDIRKKCEGRLCYDFSNMEKFLNQESVRSALGVGDIEFQSCSTTVYQALLVDWMRNLEVGIPALLEDGIQLLVYAGEYDLICNWLGNSRWVHAMEWSAQQDFVASPEIPFEVDGSEAGILKSLGPLSFLKVHDAGHMVPMDQPKAALEMLRRWTKGSLLLSTSTPQTFVASQ

SEQ ID NO: 62 - DNA, 1554 bp, Chenopodium quinoa
  source 1..1554: mol_type = genomic DNA
atgggttttaagttttttcataaatttagttattttgttgttgttttttgtctattgaatttacaatcattgtgttttctaaccaatgctaaaccaaaccataaacaacaagaacattttaagttgtcatctaccacaacatttccaaaacttcaagcagagagacttataaggcaacttaatttattcccaaaacacccttataaccaccacgacgacgtcgaaagcgggttatcggtttcggaatcgggtttggtagagaaaaggtttaagatatccgaattgggtggtgacccgggtactagtgttgaggatttgggtcatcatgcagggtattatcgtcttcctcattccaaagatgccaggatgttctacttcttttttgaatcacgaaatcgcaaggatgatcccgtcgtgatttggttgacaggaggaccagggtgcagcagtgagctagctctattctatgagaatggaccttatcaaattgccaacaatatgtctttggtttggaatgattatggttgggataaggcttccaatctcatctacgtcgaccagccaataggaacaggttttagttacacttccaattctggtgacatccgacacgatgagaatggtgttagcaatgacttgtatgacttcctgcaggctttcttcaaggcgcatcctcagtatgccaagaatgacttctatatcactggagaatcctacgctgggcattacatccctgcctttgctgctagagttcacaacggaaacaaggctaaagaaggactccccataaatctcaagggatttgcaattggtaacggcctcacccatccagaaatccaatacaaagcatacccagactttgctttggataatggaatcatcaaacaggcagagcacaaaagcatcagcgaaatggttcctgcttgtgaacaagcaatcaagcagtgtggtactgatggtaaagattcttgcttgaatgcgtactttgcttgcaccaacatcttcaacaaaatcatggactatgcaggagacacaaattactatgacatcaggaagaagtgtgagaccagcttgtgttatgacttttcgaatatggagacattactgaatcagaaatcagtaagaaaagcccttggtgttggggacattgacttcgtatcatgtagtaccgaggtgtatgatgccatgcaaatggactggatgaggaatctcgaagttggtatccctgcccttttggaggatggaatcaagctgctggtttatgctggggagtatgatctgatatgcaactggcttgggaactctaggtgggttcatgccgtggaatggtctggacagaaagattttacagcagctcctactgtatccttcactgttgataacaaagaagcaggacaaatgaaaagccatggttcccttgctttcctcaaggtgcatgatgctggacacatggtcccaatggaccagcctaaggctgctctagagatgctcaagaggtggactcaaggtactctggcttcagcagatataacagagaagatttcacctgtgtga

SEQ ID NO: 63 - protein, 517 aa, Chenopodium quinoa
MGFKFFHKFSYFVVVFCLLNLQSLCFLTNAKPNHKQQEHFKLSSTTTFPKLQAERLIRQLNLFPKHPYNHHDDVESGLSVSESGLVEKRFKISELGGDPGTSVEDLGHHAGYYRLPHSKDARMFYFFFESRNRKDDPVVIWLTGGPGCSSELALFYENGPYQIANNMSLVWNDYGWDKASNLIYVDQPIGTGFSYTSNSGDIRHDENGVSNDLYDFLQAFFKAHPQYAKNDFYITGESYAGHYIPAFAARVHNGNKAKEGLPINLKGFAIGNGLTHPEIQYKAYPDFALDNGIIKQAEHKSISEMVPACEQAIKQCGTDGKDSCLNAYFACTNIFNKIMDYAGDTNYYDIRKKCETSLCYDFSNMETLLNQKSVRKALGVGDIDFVSCSTEVYDAMQMDWMRNLEVGIPALLEDGIKLLVYAGEYDLICNWLGNSRWVHAVEWSGQKDFTAAPTVSFTVDNKEAGQMKSHGSLAFLKVHDAGHMVPMDQPKAALEMLKRWTQGTLASADITEKISPV

SEQ ID NO: 64 - DNA, 1341 bp, Chenopodium quinoa
  source 1..1341: mol_type = genomic DNA
atgaaaacattcatcctctcattatctatccttcttcttctattccattttactcctactattagttcatcatccaccccatttacacccgaatcgcttcctactaaatccggctacttagtcgtaaactcgacttctaagtcgtcgatttactacacttattatgaagcacagcaacctgtttcaacacttgaaaaaactccacttcttgtatggctccaaggaggtcctggttgttcctctatggttgggaacttctatgagctcggcccgtggcgggttaaccataagattgagttagaaagaaataaaggcgcatggaatcgaatattcgggcttttgttcttggataatccaattgggacagggtttagcattgctgctacaccagaggagattccaagggatcaacatggtgtagctaaacatctttttattgctattaaatcatttcttgcattagataaagctgtatttggaaatcgacccgtttatattactggtgaaagttatgcaggaaaatatgctcctgcaatagggtattacatagtaaggagaaatagtgagctaccagaaactgagagggtaaattttggtggaattgcaattggaaacgggttaactgacccgataacccaggtgacaacccatgggttgaatgcttattttagtgggttggtgaatgaaaagcagagattgcagttagaggaaggtcaaagggcggcggtaaagttggttatggagaggaagtggactgaggcaactgatgcaagaagtagagtattgaatttgctccaagagatgacagggttggctactttgtatgatatcagaaggaaaataccctatgagtcgtacttggtagagcggtttttgagcagttctgaggtcaagacggcgttgggggtgaataaatccatggtttgggaggactgcagtaatatagtaggcgagattttacatgaagatgtaatgaagagtgtgaagtacatggtggaaatgttgttgcagaagaccaaggttctgttatatcaggggcagtgtgacctgagagatggggtggtttccgtagaggcttggctgaagaagttgaagtgggaaggggttcggaagtttgaagatgcagagaggaaagtgtggaaagtagaagaggaggttgccgggtatgtgcagaaatgggggagtttgactcatgctgtagtgttgggagcaggacattttgtgcccactgatcaggctgttcattcgcaggtcatgatcgaggattggatccttgaaaaaggaattttcgcaagagatgaaattgagattgaatcacgatgtccaaagaattttctttga

SEQ ID NO: 65 - protein, 446 aa, Chenopodium quinoa
MKTFILSLSILLLLFHFTPTISSSSTPFTPESLPTKSGYLVVNSTSKSSIYYTYYEAQQPVSTLEKTPLLVWLQGGPGCSSMVGNFYELGPWRVNHKIELERNKGAWNRIFGLLFLDNPIGTGFSIAATPEEIPRDQHGVAKHLFIAIKSFLALDKAVFGNRPVYITGESYAGKYAPAIGYYIVRRNSELPETERVNFGGIAIGNGLTDPITQVTTHGLNAYFSGLVNEKQRLQLEEGQRAAVKLVMERKWTEATDARSRVLNLLQEMTGLATLYDIRRKIPYESYLVERFLSSSEVKTALGVNKSMVWEDCSNIVGEILHEDVMKSVKYMVEMLLQKTKVLLYQGQCDLRDGVVSVEAWLKKLKWEGVRKFEDAERKVWKVEEEVAGYVQKWGSLTHAVVLGAGHFVPTDQAVHSQVMIEDWILEKGIFARDEIEIESRCPKNFL

SEQ ID NO: 66 - DNA, 1500 bp, Chenopodium quinoa
  source 1..1500: mol_type = genomic DNA
atggctttctcctcttctacaccatcatcattaacattttcttgtaaatatttttgtttatctatactcctaacatggcttacaacccatgttatgatagtttccgcttcggtatcgacccttgggctaaccccagaggtggtggctaagcagaacgccgaccgcgtcgtcgacttgccagggcagccggtagttgggtttaagcattatgctggttatgtgaatgtgaatgctagtcatggtagagaactgttttattggttttttgaagctgatcataatcctcatcttaaacctgtggttctctggcttaatggaggtcctggatgctcttcggttggttatggtgaagctgaagagctaggtcccttcttccctaagaagggaagcaaaccgtcgttgaaacacaacaaattctcatggaataaagctgcaaatctgttgtttttggagtctcctgttggtgttggtttttcttacaccaatactagcagtgattttgatgaactaggtgacacttttacagcaaaggattcttacatattccttgtcaactggttcaagagatttccgcaatttaagtcccatgaattctatttagcaggggaaagttatgcaggccactatgttccacagctcgcagagtacatctttgacagtaaaaaacatgcttgcaaggaggattttatcaatctgaaaggtttcttgattgggaacgcgttgttggatgatgaaacagatcaaacaggaatgatcgactatgcctgggatcatgctgttatatcagataaagtcttcagagatgtgaagagaaagtgcaacttcaaagttgagaacgtatcagatgattgcacaagtgcacttaatgagtactttgaagtttacgacctgatagatatgtacagcttgtacgcgcctatctgcacaaaaaattacactgatgttaacagcagcaacagcaggaggcaactttctgccatcaaaggcattgcacctcgcgtgttctccaaatttgataaatggcacaggcggcctgctggatacgacccttgtttagatcattacacagacgtgtatctaaataggccagatgttcaagcagcacttcatgccaatacctcgaaaattccttacgcttggaccttctgcagcgacaacattacattttgggctgatgcacctgcctcaatacttcctgttattaggaagcttatagatggtgggcttcggatatgggttttcagtggagatactgatgggaggattcctgtgagttcgacgaggttaacactcagaaagcttggcttgaagatcaaacaagattggacaccctggtatagcaaagatcaggttggtggatggaccattgcctatgatgggctgatgtttgttacagttcgaggcgcgggtcaccaagttccaacttacaagccaagggaagcccttctgatgctcaggcatttccttgccaacaaaacaatgccgtctaaagccttttga

SEQ ID NO: 67 - protein, 499 aa, Chenopodium quinoa
MAFSSSTPSSLTFSCKYFCLSILLTWLTTHVMIVSASVSTLGLTPEVVAKQNADRVVDLPGQPVVGFKHYAGYVNVNASHGRELFYWFFEADHNPHLKPVVLWLNGGPGCSSVGYGEAEELGPFFPKKGSKPSLKHNKFSWNKAANLLFLESPVGVGFSYTNTSSDFDELGDTFTAKDSYIFLVNWFKRFPQFKSHEFYLAGESYAGHYVPQLAEYIFDSKKHACKEDFINLKGFLIGNALLDDETDQTGMIDYAWDHAVISDKVFRDVKRKCNFKVENVSDDCTSALNEYFEVYDLIDMYSLYAPICTKNYTDVNSSNSRRQLSAIKGIAPRVFSKFDKWHRRPAGYDPCLDHYTDVYLNRPDVQAALHANTSKIPYAWTFCSDNITFWADAPASILPVIRKLIDGGLRIWVFSGDTDGRIPVSSTRLTLRKLGLKIKQDWTPWYSKDQVGGWTIAYDGLMFVTVRGAGHQVPTYKPREALLMLRHFLANKTMPSKAF

SEQ ID NO: 68 - DNA, 1515 bp, Chenopodium quinoa
  source 1..1515: mol_type = genomic DNA
atgggttttaagttttttcataaatttagttattttgttgttgttttttgtctattgaatttacaatcattgtgttttctaaccaatgctaaaccaaaccataaacaacaagaacattttaagttgtcatctaccacaacatttccaaaacttcaagcagagagacttataaggcaacttaatttattcccaaaacacccttataaccaccacgacgacgtcgaaagcgggttatcggtttcggaatcgggtttggtagagaaaaggtttaagatatccgaattgggtggtgacccgggtactagtgttgaggatttgggtcatcatgcagggtattatcgtcttcctcattccaaagatgccaggatgttctacttcttttttgaatcacgaaatcgcaaggatgatcccgtcgtgatttggttgacaggaggaccagggtgcagcagtgagctagctctattctatgagaatggaccttatcaaattgccaacaatatgtctttggtttggaatgattatggttgggataaggcttccaatctcatctacgtcgaccagccaataggaacaggttttagttacacttccaattctggtgacatccgacacgatgagaatggtgttagcaatgacttgtatgacttcctgcaggctttcttcaaggcgcatcctcagtatgccaagaatgacttctatatcactggagaatcctacgctgggcattacatccctgcctttgctgctagagttcacaacggaaacaaggctaaagaaggactccccataaatctcaagggatttgcaattggtaacggcctcacccatccagaaatccaatacaaagcatacccagactttgctttggataatggaatcatcaaacaggcagagcacaaaagcatcagcgaaatggttcctgcttgtgaacaagcaatcaagcagtgtggtactgatggtaaagattcttgcttgaatgcgtactttgcttgcaccaacatcttcaacaaaatcatggactatgcaggagacacaaattactatgacatcaggaagaagtgtgagaccagcttgtgttatgacttttcgaatatggagacattactgaatcagaaatcagtaagaaaagcccttggtgttggggacattgacttcgtatcatgtagtaccgaggtgtatgatgccatgcaaatggactggatgaggaatctcgaagttggtatccctgcccttttggaggatggaatcaagctgctggtttatgctggggagtatgatctgatatgcaactggcttgggaactctaggtgggttcatgccgtggaatggtctggacagaaagattttacagcagctcctactgtatccttcactgttgataacaaagaagcaggacaaatgaaaagccatggttcccttgctttcctcaaggtgcatgatgctggacacatggtcccaatggaccagcctaaggctgctctagagatgctcaagaggtggactcaaggatcataa

SEQ ID NO: 69 - protein, 504 aa, Chenopodium quinoa
MGFKFFHKFSYFVVVFCLLNLQSLCFLTNAKPNHKQQEHFKLSSTTTFPKLQAERLIRQLNLFPKHPYNHHDDVESGLSVSESGLVEKRFKISELGGDPGTSVEDLGHHAGYYRLPHSKDARMFYFFFESRNRKDDPVVIWLTGGPGCSSELALFYENGPYQIANNMSLVWNDYGWDKASNLIYVDQPIGTGFSYTSNSGDIRHDENGVSNDLYDFLQAFFKAHPQYAKNDFYITGESYAGHYIPAFAARVHNGNKAKEGLPINLKGFAIGNGLTHPEIQYKAYPDFALDNGIIKQAEHKSISEMVPACEQAIKQCGTDGKDSCLNAYFACTNIFNKIMDYAGDTNYYDIRKKCETSLCYDFSNMETLLNQKSVRKALGVGDIDFVSCSTEVYDAMQMDWMRNLEVGIPALLEDGIKLLVYAGEYDLICNWLGNSRWVHAVEWSGQKDFTAAPTVSFTVDNKEAGQMKSHGSLAFLKVHDAGHMVPMDQPKAALEMLKRWTQGS